SEQUENCE LISTING (65 sequences)

SEQ ID NO: 1 - protein, 137 aa, Artificial Sequence
  Synthetic
ALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 2 - protein, 126 aa, Artificial Sequence
  Synthetic
NKLHAGSMGKKSGKKFFVTNHERMPFSKVKALCSELRGTVAIPRNAEENKAIQEVAKTSAFLGITDEVTEGQFMYVTGGRLTYSNWKKDEPNDHGSGEDCVTIVDNGLWNDISCQASHTAVCSFPA

SEQ ID NO: 3 - protein, 127 aa, Artificial Sequence
  Synthetic
KKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCAF

SEQ ID NO: 4 - protein, 123 aa, Artificial Sequence
  Synthetic
KVYWFCYGMKCYYFVMDRKTWSGCKQTCQSSSLSLLKIDDEDELKFLQLLVVPSDSCWVGLSYDNKKDWAWIDNRPSKLALNTRKYNIRDRGGCMLLSKTRLDNGNCDQVFICICGKRLDKFP

SEQ ID NO: 5 - protein, 128 aa, Artificial Sequence
  Synthetic
CPVNWVEHERSCYWFSRSGKAWADADNYCRLEDAHLVVVTSWEEQLFVQHHIGPVNTWMGLHDQNGPWKWVDGTDYETGFKNWRPEQPDDWYGHGLGGGEDCAHFTDDGRWNDDVCQRPYRWVCSTEL

SEQ ID NO: 6 - protein, 147 aa, Artificial Sequence
  Synthetic
GIPKCPEDWGASSRTSLCFKLYAKGKHEKKTWFESRDFCRALGGDLASINNKEEQQTIWRLITASGSYHKLFWLGLTYGSPSEGFTWSDGSPVSYENWAYGEPNNYQNVEYCGELKGDPTMSWNDINCEHLNNWICQIQKGQTPKPD

SEQ ID NO: 7 - protein, 129 aa, Artificial Sequence
  Synthetic
DCLSGWSSYEGHCYKAFSKYKTWEDAERVCTEQAKGAHLVSIESSGEADFVAQLVTQNMKRLDFYIWIGLRVQGKVKQCNSEWSDGSSVSYENWIEAESKTCLGLEKETDFRKWVNIYCGQQNPFVCEA

SEQ ID NO: 8 - protein, 122 aa, Artificial Sequence
  Synthetic
DCPSDWSSYEGHCYKPFSEPKNWADAENFCTQQHAGGHLVSFQSSEEADFVVKLAFQTFHSIFWMGLSNVWNQCNWQWSNAAMLRYKAWAEESYCVYFKSTNNKWRSRACRMMAQFVCEFQA

SEQ ID NO: 9 - protein, 135 aa, Artificial Sequence
  Synthetic
ARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFGKWKDVPCEDKFSFVCKFKN

SEQ ID NO: 10 - protein, 123 aa, Artificial Sequence
  Synthetic
DYEILFSDETMNYADAGTYCGSRGMALVSSAMRDSTMVKAILAFTEVKGHDYWVGADNLQDGAYNFNWNDGVSLPTDSDLWSPNEPSNPQSWQLCVQIWSKYNLLDDVGCGGARRVICEKELD

SEQ ID NO: 11 - protein, 181 aa, Artificial Sequence
  Synthetic
EPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 12 - DNA, 546 bp, Artificial Sequence
  Synthetic
gagccaccaacccagaagcccaagaagattgtaaatgccaagaaagatgttgtgaacacaaagatgtttgaggagctcaagagccgtctggacaccctggcccaggaggtggccctgctgaaggagcagcaggccctgcagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggcgcccgcatcgcctacaagaactgggagactgagatcaccgcgcaacccgatggcggcaagaccgagaactgcgcggtcctgtcaggcgcggccaacggcaagtggttcgacaagcgctgccgcgatcagctgccctacatctgccagttcgggatcgtgtag

SEQ ID NO: 13 - DNA, 546 bp, Artificial Sequence
  Synthetic
gagtcacccactcccaaggccaagaaggctgcaaatgccaagaaagatttggtgagctcaaagatgttcgaggagctcaagaacaggatggatgtcctggcccaggaggtggccctgctgaaggagaagcaggccttacagactgtgtgcctgaagggcaccaaggtgaacttgaagtgcctcctggccttcacccaaccgaagaccttccatgaggcgagcgaggactgcatctcgcaagggggcacgctgggcaccccgcagtcagagctagagaacgaggcgctgttcgagtacgcgcgccacagcgtgggcaacgatgcgaacatctggctgggcctcaacgacatggccgcggaaggcgcctgggtggacatgaccggcggcctcctggcctacaagaactgggagacggagatcacgacgcaacccgacggcggcaaagccgagaactgcgccgccctgtctggcgcagccaacggcaagtggttcgacaagcgatgccgcgatcagttgccctacatctgccagtttgccattgtgtag

SEQ ID NO: 14 - protein, 181 aa, Artificial Sequence
  Synthetic
ESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 15 - protein, 202 aa, Homo sapiens
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 16 - protein, 202 aa, Mus musculus
MGFWGTYLLFCLFSFLSQLTAESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 17 - protein, 201 aa, Gallus gallus
MALRGACLLLCLVSLAHISVQQNGKGRQKPAASKKDGVSLKMIEDLKAMIDNISQEVALLKEKQALQTVCLKGTKIHLKCFLAFSESKTYHEASEHCISQGGTLGTPQGGEENDALYDYMRKSIGNEAEIWLGLNDMVAEGKWVDMTGSPIRYKNWETEITTQPDGGKLENCAALSGVAVGKWFDKRCKEQLPYVCQFMIV

SEQ ID NO: 18 - protein, 202 aa, Bos taurus
MELWGPCVLLCLFSLLTQVTAETPTPKAKKAANAKKDAVSPKMLEELKTQLDSLAQEVALLKEQQALQTVCLKGTKVHMKCFLAFVQAKTFHEASEDCISRGGTLGTPQTGSENDALYEYLRQSVGSEAEVWLGFNDMASEGSWVDMTGGHIAYKNWETEITAQPDGGKVENCATLSGAANGKWFDKRCRDKLPYVCQFAIV

SEQ ID NO: 19 - protein, 198 aa, Salmo salar
MRVSGVRLLFCLLLLGQSTFQQTSSKKKGGKKDAENNAAIEELKKQIDNIVLELNLLKEQQALQSVCLKGIKIIGKCFLADTAKKIYHTAYDDCIAKGGTISTPLTGDENDQLVDYVRRSIGPEEHIWLGINDMVTEGEWLDQAGTNLRFKNWETDITNQPDGGRTHNCAILSTTANGKWFDESCRVEKASVCEFNIV

SEQ ID NO: 20 - protein, 198 aa, Silurana tropicalis
MEYRRACILLCLFCFVQVTLQQNGKKNKQNNKDVVSMKMYEDLKKKVQNIEEDVIHLKEQQALQTICLKGMKIYNKCFLAFNELKTYHQASDVCFAQGGTLSTPETGDENDSLYDYVRKSIGSSAEIWIGINDMATEGTWLDLTGSPISFKHWETEITTQPDGGKQENCAALSASAIGRWFDKNCKTELPFVCQFSIV

SEQ ID NO: 21 - protein, 223 aa, Danio rerio
MRDDSDKVPSLLTDYILKGCTYAEEKMDLKAVKFLLCVICLVKSSPEQSLTKRKNGKKESNSAAIEELKKQIDQIIQDLNLLKEQQALQTVCLKGFKIPGKCFLVDTVKKDFHSANDDCIAKGGILSTPMSGHENDQLQEYVQQTVGPETHIWLGVNDMIKEGEWIDLTGSPIRFKNWESEITHQPDGGRTHNCAVLSSTANGKWFDEDCRGEKASVCQFNIV

SEQ ID NO: 22 - protein, 197 aa, Bos taurus
MAKNGLVIYILVITLLLDQTSCHASKFKARKHSKRRVKEKDGDLKTQVEKLWREVNALKEMQALQTVCLRGTKFHKKCYLAAEGLKHFHEANEDCISKGGTLVVPRSADEINALRDYGKRSLPGVNDFWLGINDMVAEGKFVDINGLAISFLNWDQAQPNGGKRENCALFSQSAQGKWSDEACHSSKRYICEFTIPQ

SEQ ID NO: 23 - protein, 166 aa, Carcharhinus springeri
SKPSKSGKGKDDLRNEIDKLWREVNSLKEMQALQTVCLKGTKIHKKCYLASRGSKSYHAANEDCIAQGGTLSIPRSSDEGNSLRSYAKKSLVGARDFWIGVNDMTTEGKFVDVNGLPITYFNWDRSKPVGGTRENCVAASTSGQGKWSDDVCRSEKRYICEYLIPV

SEQ ID NO: 24 - protein, 204 aa, Artificial Sequence
  Synthetic
MELWGAXXLLCLFSXLXQVTAXXXXXKAKKXXXXXKKDXVSXKMXEELKXQIDXLAQEVXLLKEQQALQTVCLKGTKIHXKCFLAFTQXKTFHEASEDCISQGGTLSTPQXGDENDALXXYXRXSVGNEAXIWLGXNDMAAEGXWVDMTGSXIXYKNWETEITXQPDGGKXENCAALSXXANGKWFDKXCRDELPYVCQFXIVX

SEQ ID NO: 25 - protein, 125 aa, Artificial Sequence
  Synthetic
HMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 26 - protein, 114 aa, Artificial Sequence
  Synthetic
GNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 27 - protein, 112 aa, Artificial Sequence
  Synthetic
KYFMSSVRRMPLNRAKALCSELQGTVATPRNAEENRAIQNVAKDVAFLGITDQRTENVFEDLTGNRVRYTNWNEGEPNNVGSGENCVVLLTNGKWNDVPCSDSFLVVCEFSD

SEQ ID NO: 28 - protein, 115 aa, Artificial Sequence
  Synthetic
GEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 29 - protein, 114 aa, Artificial Sequence
  Synthetic
GKKFFVTNHERMPFSKVKALCSELRGTVAIPRNAEENKAIQEVAKTSAFLGITDEVTEGQFMYVTGGRLTYSNWKKDEPNDVGSGEDCVTIVDNGLWNDVSCQASHTAVCEFPA

SEQ ID NO: 30 - protein, 114 aa, Artificial Sequence
  Synthetic
GDKVFSTNGQSVNFDTIKEMCTRAGGNIAVPRTPEENEAIASIAKKYNNYVYLGMIEDQTPGDFHYLDGASVSYTNWYPGEPRGQGKEKCVEMYTDGTWNDRGCLQYRLAVCEF

SEQ ID NO: 31 - protein, 119 aa, Artificial Sequence
  Synthetic
TKFQGHCYRHFPDRETWVDAERRCREQQSHLSSIVTPEEQEFVNKNAQDYQWIGLNDRTIEGDFRWSDGHSLQFEKWRPNQPDNFFATGEDCVVMIWHERGEWNDVPCNYQLPFTCKKG

SEQ ID NO: 32 - protein, 127 aa, Artificial Sequence
  Synthetic
SHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 33 - protein, 119 aa, Artificial Sequence
  Synthetic
DKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDR

SEQ ID NO: 34 - protein, 120 aa, Artificial Sequence
  Synthetic
DKCYYFSLEKEIFEDAKLFCEDKSSHLVFINSREEQQWIKKHTVGRESHWIGLTDSEQESEWKWLDGSPVDYKNWKAGQPDNWGSGHGPGEDCAGLIYAGQWNDFQCDEINNFICEKERE

SEQ ID NO: 35 - protein, 121 aa, Artificial Sequence
  Synthetic
GNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFR

SEQ ID NO: 36 - DNA, 240 bp, Artificial Sequence
  Synthetic
gaggccgagatctggctgggcctgaacgacatgnnknnknnknnknnknnknnktgggtggatatgactggcgcccgcatcgcctacaagaactgggaaactgagatcaccgcccaacctgatggcggcgcaaccgagaactgcgcggtcctgtctggcgccgccaacggcaagtggttcgacaagcgctgcagggatcaattgccctacatctgccagttcgggatcgtggcggccgca

SEQ ID NO: 37 - protein, 80 aa, Artificial Sequence
  Synthetic
EAEIWLGLNDMXXXXXXXWVDMTGARIAYKNWETEITAQPDGGATENCAVLSGAANGKWFDKRCRDQLPYICQFGIVAAA

SEQ ID NO: 38 - protein, 137 aa, Artificial Sequence; identical to SEQ ID NO: 1
  Synthetic

SEQ ID NO: 39 - DNA, 414 bp, Artificial Sequence
  Synthetic
caggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgactggcgcgcgtatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtg

SEQ ID NO: 40 - protein, 52 aa, Artificial Sequence
  Synthetic
EPPTQKPKKIVNAKKDVVNTKMFEELKSRLDTLAQEVALLKEQQALQTVCLK

SEQ ID NO: 41 - protein, 52 aa, Artificial Sequence
  Synthetic
ESPTPKAKKAANAKKDLVSSKMFEELKNRMDVLAQEVALLKEKQALQTVCLK

SEQ ID NO: 42 - protein, 52 aa, Artificial Sequence
  Synthetic
QQNGKGRQKPAASKKDGVSLKMIEDLKAMIDNISQEVALLKEKQALQTVCLK

SEQ ID NO: 43 - protein, 52 aa, Artificial Sequence
  Synthetic
ETPTPKAKKAANAKKDAVSPKMLEELKTQLDSLAQEVALLKEQQALQTVCLK

SEQ ID NO: 44 - protein, 49 aa, Artificial Sequence
  Synthetic
QQTSSKKKGGKKDAENNAAIEELKKQIDNIVLELNLLKEQQALQSVCLK

SEQ ID NO: 45 - protein, 49 aa, Artificial Sequence
  Synthetic
QQNGKKNKQNNKDVVSMKMYEDLKKKVQNIEEDVIHLKEQQALQTICLK

SEQ ID NO: 46 - protein, 48 aa, Artificial Sequence
  Synthetic
EQSLTKRKNGKKESNSAAIEELKKQIDQIIQDLNLLKEQQALQTVCLK

SEQ ID NO: 47 - protein, 52 aa, Artificial Sequence
  Synthetic
QTSCHASKFKARKHSKRRVKEKDGDLKTQVEKLWREVNALKEMQALQTVCLR

SEQ ID NO: 48 - protein, 38 aa, Artificial Sequence
  Synthetic
KPSKSGKGKDDLRNEIDKLWREVNSLKEMQALQTVCLK

SEQ ID NO: 49 - protein, 52 aa, Artificial Sequence
  Synthetic
XXXXXXXXXXXXXXXXXXXXXXXXXLXXXXXXLXXEVXXLKEXQALQTVCLX

SEQ ID NO: 50 - DNA, 4779 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctaagtgacgatatcctgacctaaggtacctaagtgacgatatcctgacctaactgcagggatcaattgccctacatctgccagttcgggatcgtggcggccgcaggtgcgccggtgccgtatccggatccgctggaaccgcgtgccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 51 - DNA, 5747 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgggatctcgatcccgcgaaattaatacgactcactataggggaattgtgagcggataacaattcccctctagaaataattttgtttaactttaagaaggagatatacatatgaaataccttcttccgactgctgctgctggtcttttactgctggctgctcagccggctatggctgctggtggtggttctgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 52 - DNA, 10975 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 53 - DNA, 5774 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgggatctcgatcccgcgaaattaatacgactcactataggggaattgtgagcggataacaattcccctctagaaataattttgtttaactttaagaaggagatatacatatgaaataccttcttccgactgctgctgctggtcttttactgctggctgctcagccggctatggctgctggtggtggttctgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgcccaccaccaccaccaccactaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 54 - DNA, 4649 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttctgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 55 - DNA, 10972 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacgtgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 56 - DNA, 10972 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccaggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 57 - DNA, 10969 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagtgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 58 - DNA, 10975 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtcagcctgaaggggaccaaggtgcacatgaaaagctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 59 - DNA, 10927 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgcaccaccaccaccaccactaactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 60 - DNA, 4641 bp, Artificial Sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattcaccatgaaaaagacagctatcgcgattgcagtggcactggctggtttcgctaccgttgcgcaagcttctgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccgcagttcgaaaaataactcgagataaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 61 - DNA, 11011 bp, Artificial Sequence
  Synthetic
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctgggtaccagctgctagcgtttaaacttaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagctgtggggggcctacctgctgctgtgcctgttctccctgctgacccaggtgaccaccgagccaccaacccagaagcccaagaagattgtaaatgccaagaaagatgttgtgaacacaaagatgtttgaggagctcaagagccgtctggacaccctggcccaggaggtggccctgctgaaggagcagcaggccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccccagttcgagaagtgactcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaagaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgccgaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatccatacattgaatcaatattggcaattagccatattagtcattggttatatagcataaatcaatattggctattggccattgcatacgttgtatctatatcataatatgtacatttatattggctcatgtccaatatgaccgccat

SEQ ID NO: 62 - DNA, 5783 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgggatctcgatcccgcgaaattaatacgactcactataggggaattgtgagcggataacaattcccctctagaaataattttgtttaactttaagaaggagatatacatatgaaataccttcttccgactgctgctgctggtcttttactgctggctgctcagccggctatggctgctggtggtggttctgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctggctgggcctcaacgacatggcggccgagggcacctgggtggacatgaccggtacccgcatcgcctacaagaactgggagactgagatcaccgcgcaacccgatggcggcaagaccgagaactgcgcggtcctgtcaggcgcggccaacggcaagtggttcgacaagcgctgcagggatcaattgccctacatctgccagttcgggatcgtgtacccctacgacgtgcccgactacgccggttggagccacccgcagttcgaaaaataactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 63 - DNA, 4792 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccttacagactgtgtgcctgaagggcaccaaggtgaacttgaagtgcctcctggccttcacccaaccgaagaccttccatgaggcgagcgaggactgcatctcgcaagggggcacgctgggtaccccgcagtcagagctggagaacgaggcgctgttcgaatacgcgcgccacagcgtgggcaacgatgcgaacatctggctgggcctcaacgacatggccgcggaaggcgcctgggtcgactaagtgatatcctgacctaactgcagagatcagttgccctacatctgccagtttgccattgtggcggccgcaggtgcgccggtgccgtatccggatccgctggaaccgcgtgccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 64 - DNA, 4101 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccctccagacggtctgcctgaaggggaccaaggtgcacatgaaatgctttctggccttcacccagacgaagaccttccacgaggccagcgaggactgcatctcgcgcgggggcaccctgagcacccctcagactggctcggagaacgacgccctgtatgagtacctgcgccagagcgtgggcaacgaggccgagatctaagtgacgatatcctgacctaaggtacctaagtgacgatatcctgacctaactgcagggatcaattgccctacatctgccagttcgggatcgtggcggccgcaggtgcgccggtgccgtatccggatccgctggaaccgcgtgccgcacaggctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 65 - DNA, 4114 bp, Artificial Sequence
  Synthetic
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggccgccttacagactgtgtgcctgaagggcaccaaggtgaacttgaagtgcctcctggccttcacccaaccgaagaccttccatgaggcgagcgaggactgcatctcgcaagggggcacgctgggtaccccgcagtcagagctggagaacgaggcgctgttcgaatacgcgcgccacagcgtgggcaacgatgcgaacatctggctgggcctcaacgacatggccgcggaaggcgcctgggtcgactaagtgatatcctgacctaactgcagagatcagttgccctacatctgccagtttgccattgtggcggccgcaggtgcgccggtgccgtatccggatccgctggaaccgcgtgccgcacaggctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

(19) United States
(12) Patent Application Publication
(10) Pub. No.: US 2011/0086770 A1
(43) Pub. Date: Apr. 14, 2011

(54) Combinatorial Libraries Based on C-type Lectin-like Domain

(75) Inventors: Martha Wild, San Diego, CA (US); Anke Kretz-Rommel, San Diego, CA (US); Katherine S. Bowdish, Del Mar, CA (US); Mark Renshaw, San Diego, CA (US)

(73) Assignee: Anaphore, Inc.

(21) Appl. No.: 12/703,752

(22) Filed: Feb. 10, 2010

Related U.S. Application Data
(63) Continuation-in-part of application No. 12/577,067, filed on Oct. 9, 2009.
Application No. 12/577,067 is a continuation-in-part of application No. PCT/US09/60271, filed on Oct. 9, 2009.

(51) Int. Cl. C40B 30/04; C40B 40/10; C40B 40/08; C40B 40/02; C40B 50/00; C40B 50/06; C07K 2/00; C07K 1/00

(52) U.S. Cl. 506/9; 506/18; 506/17; 506/14; 506/23; 506/26; 530/300; 530/333

(57) ABSTRACT

This invention relates to polypeptide libraries comprising polypeptides having a C-type lectin domain (CTLD) with a randomized loop region, as well as nucleic acid libraries comprising nucleic acid molecules encoding such polypeptides. The invention also relates to methods for generating the randomized polypeptides and the polypeptide libraries. The invention further relates to methods of screening the polypeptide and nucleic acid libraries based on the specific binding of the modified CTLDs to a target molecule of interest. The invention also relates to polypeptides derived from such libraries that bind to target molecules of interest.

### CROSS-REFERENCE TO RELATED APPLICATION

This application is a continuation-in-part of U.S. application Ser. No. 12/577,067, filed on Oct. 9, 2009 and a continuation-in-part of International Application PCT US09/60271, filed on Oct. 9, 2009, all of which applications are incorporated by reference herein in their entireties.

### SEQUENCE LISTING STATEMENT

The sequence listing is filed in this application in electronic format only and is incorporated by reference herein. The sequence listing text file “09-493_Substitute SeqList.txt” was created on Mar. 19, 2010, and is 385 kilobytes in size.

### FIELD OF THE INVENTION

This invention relates to polypeptide libraries comprising polypeptides having a C-type lectin domain (CTLD) with a randomized loop region, as well as nucleic acid libraries comprising nucleic acid molecules encoding such polypeptides. The invention also relates to methods for generating the randomized polypeptides and the polypeptide libraries. The invention further relates to methods of screening the polypeptide and nucleic acid libraries based on the specific binding of the modified CTLDs to a target molecule of interest. The invention also relates to polypeptides derived from such libraries that bind to target molecules of interest.

### BACKGROUND OF THE INVENTION

The C-type lectin-like domain (CTLD) is a protein motif that has been identified in a number of proteins isolated from a variety of animal species (reviewed in Drickamer and Taylor (1993) and Drickamer (1999)). Initially, the CTLD domain was identified as a domain common to the so-called C-type lectins (calcium-dependent carbohydrate binding proteins) and named “Carbohydrate Recognition Domain” (“CRD”). More recently, it has become evident that this domain is shared among many eukaryotic proteins, of which several do not bind sugar moieties, and hence, the canonical domain has been named “CTLD.”

CTLDs have been reported to bind a wide diversity of compounds, including carbohydrates, lipids, proteins, and even ice (Aspberg et al. (1997), Bettler et al. (1992), Ewart et al. (1998), Graversen et al. (1998), Mizumo et al. (1997), Sano et al. (1998), and Tormo et al. (1999)). While some proteins contain a single copy of the CTLD, other proteins contain from two to multiple copies of the domain. In the physiologically functional unit, multiplicity in the number of CTLDs is often achieved by assembling single copy protein protomers into larger structures.

The CTLD contains approximately 120 amino acid residues and, characteristically, contains two or three intra-chain disulfide bridges. Although the primary sequences of CTLDs from different proteins share relatively low amino acid sequence homology, the secondary and tertiary structures of a number of CTLDs are similar, resulting in a highly conserved three dimensional structure, in which the structural variability is essentially confined to the CTLD loop-region. The CTLD loop region, which typically contains up to five loops, plays a role in ligand and calcium binding. Several CTLDs contain either one or two binding sites for calcium and most of the side chains which interact with calcium are located in the loop-region.

Based on available three-dimensional structural information, the canonical CTLD is characterized by seven main secondary structure elements (five β-strands and two α-helices) sequentially appearing in the following order: β1; α1; α2; β2; β3; β4; and β5 (FIG. 1). In CTLDs for which the three dimensional structures have been determined, the β-strands are arranged in two anti-parallel β-sheets, one composed of β1 and β5, the other composed of β2, β3 and β4. An additional β-strand, β0, often precedes β1 in the sequence and, where present, forms an additional strand integrating with the β1, β5 sheet. Further, two disulfide bridges, one connecting α1 and β5 (C_{I}-C_{IV}, FIG. 1) and one connecting β3 and the polypeptide segment connecting β4 and β5 (C_{II}-C_{III}, FIG. 1) are invariantly found in all CTLDs characterized so far.

In the CTLD three-dimensional structure, the conserved secondary and tertiary structural elements form a compact scaffold for a number of loops, which in the present context collectively are referred to as the “loop-region,” protruding out from the core. The primary structure of the loop region of the CTLDs is organized into two segments, loop segment A (LSA) and loop segment B (LSB). LSA represents the long polypeptide segment connecting β2 and β3 which often lacks regular secondary structure and contains up to four loops. LSB represents the polypeptide segment connecting the β-strands β3 and β4. A schematic of a CTLD, including the loop region, is shown in FIGS. 4-6. Residues in LSA, together with single residues in β4, have been shown to specify the Ca²⁺- and ligand-binding sites of several CTLDs, including that of tetranectin. For example, mutagenesis studies, involving substitution of a single or a few residues, have shown that changes in binding specificity, Ca²⁺-sensitivity and/or affinity can be accommodated by CTLD domains (Weis and Drickamer (1996), Chiba et al. (1999), Graversen et al. (2000)).

Tetranectin is a trimeric glycoprotein (Holtet et al. (1997), Nielsen et al. (1997)) which has been isolated from human plasma and found to be present in the extracellular matrix in certain tissues. Tetranectin is known to bind calcium, complex polysaccharides, plasminogen, fibrinogen/fibrin, and apolipoprotein (a). The interaction with plasminogen and apolipoprotein (a) is mediated by the kringle 4-protein domain therein. This interaction is known to be sensitive to calcium and to derivatives of the amino acid lysine (Graversen et al. (1998)).

A human tetranectin gene has been characterized, and both human and murine tetranectin cDNA clones have been isolated. The mature protein of both the human and murine tetranectin comprises 181 amino acid residues. See US Patent Application Publication 2007/0154901, which is incorporated here in its entirety. The three dimensional structures of full length recombinant human tetranectin and of the isolated tetranectin CTLD have been determined independently in two separate studies (Nielsen et al. (1997) and Kastrup et al. (1998)). Tetranectin is a two- or possibly three-domain protein, i.e. the main part of the polypeptide chain comprises the CTLD (amino acid residues Gly53 to Val181), whereas the region Leu26 to Lys52 encodes an alpha-helix governing trimerization of the protein via the formation of a homotrimeric parallel coiled coil. The polypeptide segment Glu1 to Glu25 contains the binding site for complex polysaccharides (Lys6 to Lys15)(Lorentsen et al. (2000)) and appears to contribute to stabilization of the trimeric structure (Holtet et al. (1997)). The two amino acid residues Lys148 and Glu150, localized in loop 4, and Asp165 (localised in β4) have been shown to be of critical importance for plasminogen kringle 4 binding, with residues Ile140 (in loop 3) and Lys166 and Arg167 (in β4) shown to be of importance as well (Graversen et al. (1998)). Substitution of Thr149 (in loop 4) with an aromatic residue has been shown to significantly increase affinity of tetranectin to kringle 4 and to increase affinity for plasminogen kringle 2 to a level comparable to the affinity of wild type tetranectin for kringle 4 (Graversen et al. (2000)). Trimerizable truncations of tetranectin have been described. See US 2010/0028995, filed Apr. 8, 2009, which is incorporated by reference herein in its entirety.

A number of other proteins having CTLDs are known, including the following non-limiting examples: lithostatin, mouse macrophage galactose lectin, Kupffer cell receptor, chicken neurocan, perlucin, asialoglycoprotein receptor, cartilage proteoglycan core protein, IgE Fc receptor, pancreatitis-associated protein, mouse macrophage receptor, Natural Killer group, stem cell growth factor, factor IX/X binding protein, mannose binding protein, bovine conglutinin, bovine CL43, collectin liver 1, surfactant protein A, surfactant protein D, e-selectin, tunicate c-type lectin, CD94 NK receptor domain, LY49A NK receptor domain, chicken hepatic lectin, trout c-type lectin, HIV gp 120-binding c-type lectin, dendritic cell immunoreceptor, and many snake venom proteins.

The variation of binding site configuration among naturally occurring CTLDs shows that their common core structure can accommodate many essentially different configurations of the ligand binding site (see, e.g., US 2007/0275393, which is incorporated by reference herein). CTLDs are therefore particularly well suited to serve as a basis for constructing new and useful protein products with desired binding properties to target molecules of interest.

For example, the CTLDs (or CTLD-based protein products) have advantages relative to antibody derivatives as each binding site in a CTLD-based protein product is harbored in a single structurally autonomous protein domain. Also, the CTLD domains are resistant to proteolysis, and neither stability nor access to the ligand-binding site is compromised by the attachment of other protein domains to the N- or C-terminus of the CTLD.

With respect to therapeutic uses, the CTLD-based protein products are identical to the corresponding natural CTLD protein already present in the body, and are therefore expected to elicit minimal immunological response in the patient. Single CTLDs are about half the mass of an antibody and may in some applications be advantageous as it may provide better tissue penetration and distribution, as well as a shorter half-life in circulation. Multivalent formats of CTLD proteins may provide increased binding capacity and avidity and longer circulation half-life.

The present invention provides combinatorial CTLD polypeptide libraries and methods for identifying and isolating CTLDs to serve as a basis for constructing new and useful protein products with desired binding properties to target molecules of interest.

### SUMMARY OF THE INVENTION

In one aspect, the invention provides a combinatorial polypeptide library comprising polypeptide members having a C-type lectin domain (CTLD) with a randomized loop region, in which the randomized loop region has been modified from the native sequence of the CTLD. The invention provides a combinatorial polypeptide library, and a library of nucleic acids encoding the library of polypeptides, comprising polypeptide members having a C-type lectin domain (CTLD) with a randomized loop region, wherein the loop region of the CTLD is randomized according to one of the following Schemes:

(a) amino acid modifications in at least one of the four loops in loop segment A (LSA) of the CTLD, wherein the amino acid modifications comprise an insertion of at least one amino acid in Loop 1 and random substitution of at least five amino acids within Loop 1;

(b) amino acid modifications in at least one of the four loops in loop segment A (LSA) of the CTLD, wherein the amino acid modifications comprise random substitution of at least five amino acids within Loop 1 and random substitution of at least three amino acids within Loop 2;

(c) amino acid modifications in at least one of the four loops in the loop segment A (LSA) of the CTLD, wherein the amino acid modifications comprise random substitution of at least seven amino acids within Loop 1 and at least one amino acid insertion in Loop 4;

(d) amino acid modifications in at least one of the four loops in the loop segment A (LSA) of the CTLD, wherein the amino acid modifications comprise at least one amino acid insertion in Loop 3 and random substitution of at least three amino acids within Loop 3;

(e) amino acid modifications in at least one of the four loops in the loop segment A (LSA) of the CTLD, wherein the amino acid modifications comprise a modification that combines two loops into a single loop, wherein the two combined loops are Loop 3 and Loop 4;

(f) amino acid modifications in at least one of the four loops in the loop segment A (LSA) of the CTLD, wherein the amino acid modifications comprise at least one amino acid insertion in Loop 4 and random substitution of at least three amino acids within Loop 4;

(g) amino acid modifications in at least one of the four loops in the loop segment A (LSA) of the CTLD and in loop segment B (LSB), wherein the amino acid modifications comprise random substitution of at least five amino acid residues in Loop 3 and random substitution of at least three amino acids within Loop 5;

(h) amino acid modifications in at least one of the four loops in the loop segment A (LSA) of the CTLD, wherein the amino acid modifications comprise random substitution of at least one amino acid and insertion of at least six amino acids in Loop 3;

(i) amino acid modifications in at least one of the four loops in the loop segment A (LSA) of the CTLD, wherein the amino acid modifications comprise a mixture of (1) random substitution of at least six amino acids in Loop 3 and (2) random substitution of at least six amino acids and at least one amino acid insertion in Loop 3; and

(j) amino acid modifications in at least one of the four loops in the loop segment A (LSA) of the CTLD, wherein the amino acid modifications comprise at least four or more amino acid insertions in at least one of the four loops in the loop segment A (LSA) or loop 5 in loop segment B (LSB) of the CTLD.

In one aspect, the CTLD of the polypeptides of the library have the following secondary structure:
a. five β-strands and two α-helices sequentially appearing in the order β1, α1, α2, β2, β3, β4, and β5, the β-strands being arranged in two anti-parallel β-sheets, one composed of β1 and β5, the other composed of β2, β3 and β4,
b. at least two disulfide bridges, one connecting α1 and β5 and one connecting β3 and the polypeptide segment connecting β4 and β5, and
c. a loop region containing loop segment A (LSA) and loop segment B (LSB) in which LSA connects β2 and β3, and LSB connects β3 and β4.

In various further aspects, the polypeptides of the library have a random substitution of the amino acid located adjacent the C-terminal end of Loop 2 in the C-terminal direction. Also, when the CTLD is from human tetranectin, the CTLD can further comprise random substitution of Arginine-130. Also, when the CTLD is from mouse tetranectin, the CTLD can further comprise random substitution of Leucine-130. In certain of the modifications of (a)-(j), when the CTLD is from human or mouse tetranectin, the CTLD can further comprise a random substitution of proline 144.

In various further embodiments, the polypeptides of the library can have random substitution of one or more amino acids involved in calcium coordination and/or plasminogen binding. For example, when the CTLD is from tetranectin, the CTLD can further comprise substitution of Lysine-148 to Alanine (in Loop 4).

In certain embodiments, when the combinatorial library has the modified CTLD of Scheme (a), the amino acid modifications comprise two amino acid insertions in Loop 1 and random substitution of at least five amino acids within Loop 1. In other embodiments, when the combinatorial library has the modified CTLD of scheme (a) and the CTLD is from human tetranectin, the amino acid modifications comprise at least one amino acid insertion in Loop 1, random substitution of at least five amino acids within Loop 1, and include a random substitution of Arginine 130. In one specific embodiment, when the combinatorial library has the modified CTLD of scheme (a) and the CTLD is from human tetranectin, the amino acid modifications comprise two amino acid insertions in Loop 1, random substitution of five amino acids within Loop 1, and a random substitution of Arginine 130. In one specific embodiment, when the combinatorial library has the modified CTLD of scheme (a) and the CTLD is from mouse tetranectin, the amino acid modifications comprise two amino acid insertions in Loop 1, random substitution of five amino acids within Loop 1, and a random substitution of Leucine 130. In any of the embodiments for scheme (a), the amino acid modifications can further comprise a substitution of Lysine-148 to Alanine.

In certain embodiments, when the combinatorial library has the modified CTLD of Scheme (b) and the CTLD is from human tetranectin, the amino acid modifications include random substitutions of at least five amino acids in Loop 1, random substitution of at least three amino acids in Loop 2, and include a random substitution of Arginine 130. In one embodiment, when the combinatorial library has the modified CTLD of Scheme (b) and the CTLD is from human tetranectin, the amino acid modifications include random substitutions of five amino acids in Loop 1, random substitution of three amino acids in Loop 2, and a random substitution of Arginine 130. In certain other embodiments, when the combinatorial library has the modified CTLD of Scheme (b) and the CTLD is from mouse tetranectin, the amino acid modifications include random substitutions of at least five amino acids in Loop 1, random substitution of at least three amino acids in Loop 2, and include a random substitution of Leucine 130. In one embodiment, when the combinatorial library has the modified CTLD of Scheme (b) and the CTLD is from mouse tetranectin, the amino acid modifications include random substitutions of five amino acids in Loop 1, random substitution of three amino acids in Loop 2, and a random substitution of Leucine 130. In any of the embodiments for scheme (b), the amino acid modifications can further comprise a substitution of Lysine-148 to Alanine. In other specific embodiments, individual members of the combinatorial library include loop regions including any or all of the polyeptpide sequences provided by Table 3 in the Examples below.

In certain embodiments, when the combinatorial library has the modifications of Scheme (c), the amino acid modifications optionally further comprise random substitution of at least two amino acids. In certain other embodiments, when the combinatorial library has the modifications of Scheme (c), the amino acid modifications comprise three amino acid insertions within Loop 4 and optionally further comprise random substitution of at least two amino acids. In one embodiment, the amino acid modifications comprise random substitution of at least seven amino acids within Loop 1, at least three amino acid insertions in Loop 4, and random substitution of at least two amino acids within Loop 4. In one specific embodiment, the amino acid modifications comprise random substitution of seven amino acids within Loop 1, three amino acid insertions in Loop 4, and random substitution of two amino acids within Loop 4. In other specific embodiments, individual members of the combinatorial library include loop regions including any or all of the polyeptpide sequences provided by Table 3 in the Examples below.

In other embodiments, when the combinatorial library has the modified CTLD of Scheme (d), the amino acid modifications can further comprise at least one amino acid insertion in Loop 4, and can further comprise random substitution of at least three amino acids within Loop 4. In any of the described embodiments for scheme (d), the amino acid modifications can comprise three amino acid insertions in Loop 3. In any of the described embodiments for scheme (d), the amino acid modifications can comprise three amino acid insertions in Loop 4. Thus, in certain embodiments, the amino acid modifications comprise random substitution of at least three amino acids within Loop 3, random substitution of at least three amino acids within Loop 4, at least one amino acid insertion in Loop 3 and at least one amino acid insertion in Loop 4. In certain embodiments, the amino acid modifications comprise random substitution of at least three amino acids within Loop 3, random substitution of at least three amino acids within Loop 4, at least three amino acid insertions in Loop 3 and at least three amino acid insertions in Loop 4. In one specific embodiment, the amino acid modifications comprise random substitution of three amino acids within Loop 3, random substitution of three amino acids within Loop 4, three amino acid insertions in Loop 3, and three amino acid insertions in Loop 4. In other specific embodiments, individual members of the combinatorial library include loop regions including any or all of the polyeptpide sequences provided by Table 3 in the Examples below.

In certain embodiments, when the members of the combinatorial library have the modified CTLD of Scheme (e), the amino acid modifications comprise random substitution of at least six amino acids within Loop 3 and random substitution of at least four amino acids within Loop 4. In one specific embodiment, the amino acid modifications comprise random substitution of six amino acids within Loop 3 and random substitution of four amino acids within Loop 4. In any of the embodiments for scheme (e), when the CTLD is from human tetranectin, the amino acid modifications can further comprise random substitution of Proline-144. In one specific embodiment, when the CTLD is from human tetranectin, the amino acid modifications comprise random substitution of six amino acids within Loop 3, random substitution of four amino acids within Loop 4, and a random substitution of proline 144, resulting in a combined Loop 3 and Loop 4 amino acid sequence, comprising, for example, NWEXXXXXXX XGGXXXN (SEQ ID NO: 578), wherein X is any amino acid and wherein the amino acid sequence of SEQ ID NO: 578 forms a single Loop region. In other specific embodiments, individual members of the combinatorial library include loop regions including any or all of the polyeptpide sequences provided by Table 3 in the Examples below.

In other embodiments, when the combinatorial library has the modified CTLD of Scheme (f), the amino acid modifications comprise four amino acid insertions in Loop 4. In one embodiment, when the combinatorial library has the modified CTLD of Scheme (f), the amino acid modifications comprise at least four amino acid insertions in Loop 4 and random substitution of at least three amino acids within Loop 4. In one specific embodiment, the amino acid substitutions comprise four amino acid insertions in Loop 4 and random substitution of three amino acids within Loop 4. In other specific embodiments, individual members of the combinatorial library include loop regions including any or all of the polyeptpide sequences provided by Table 3 in the Examples below.

In other embodiments, when the combinatorial library has the modified CTLD of Scheme (g), and the CTLD is from tetranectin, the amino acid modifications can further comprise one or more amino acid modifications in Loop 4 that modulates plasminogen binding affinity of the CTLD, for example, the substitution of Lysine 148 to Alanine. Thus, in certain embodiments, when the CTLD is from human or mouse tetranectin, the amino acid modifications comprise random substitution of at least five amino acid residues in Loop 3, random substitution of at least three amino acid residues in Loop 5, and substitution of Lysine 148 to Alanine in Loop 4. In one specific embodiment, the amino acid modifications comprises random substitution of five amino acid residues in Loop 3 and random substitution of three amino acid residues in Loop 5, and, in another specific embodiment, when the CTLD is from human or mouse tetranectin, the amino acid modifications further comprise substitution of Lysine 148 to Alanine in Loop 4. In other specific embodiments, individual members of the combinatorial library include loop regions including any or all of the polyeptpide sequences provided by Table 3 in the Examples below.

In certain embodiments, when the combinatorial library has the modified CTLD of Scheme (h) and the CTLD is from tetranectin, the amino acid modifications can further comprise one or more amino acid modifications in Loop 4 that modulates plasminogen binding affinity of the CTLD, for example, the substitution of lysine 148 to Alanine. In certain embodiments when the CTLD is from human or mouse tetranectin, the members of the combinatorial library have random substitution of at least one amino acid and insertion of at least six amino acids in Loop 3, and substitution of Lysine 148 to Alanine in Loop 4. In one specific embodiment, when the combinatorial library has the modified CTLD of Scheme (h), the amino acid modifications comprise random substitution of one amino acid and insertion of six amino acids in Loop 3. In one specific embodiment, when the CTLD is from human or mouse tetranectin, the members of the combinatorial library have random substitution of one amino acid and insertion of six amino acids in Loop 3, and substitution of lysine 148 to alanine in Loop 4. In any of these embodiments when the CTLD is from human or mouse tetranectin, one of the substitutions is the substitution of Isoleucine 140. In other specific embodiments, individual members of the combinatorial library include loop regions including any or all of the polyeptpide sequences provided by Table 3 in the Examples below.

In one embodiment, when the combinatorial library has the modified CTLD of Scheme (i), the amino acid modifications comprise a mixture of random substitution of six amino acids in Loop 3, random substitution of six amino acids and one amino acid insertion in Loop 3, and random substitution of six amino acids and two amino acid insertions in Loop 3. In any of the embodiments of scheme (i), when the CTLD is from tetranectin, the amino acid modifications further comprise a substitution of Lysine 148 to Alanine in Loop 4.

In further aspects of the invention, the polypeptide members of the combinatorial polypeptide library have one or more amino acid modifications in any combination of two, three, four, or five of the loops in loop segment A (LSA) and loop segment B (LSB). The polypeptide members can also comprise a CTLD region having amino acid modifications in regions outside of the LSA and LSB. In other specific embodiments, individual members of the combinatorial library include loop regions including any or all of the polyeptpide sequences provided by Table 17 in the Examples below.

In certain embodiments of the invention, the combinatorial library is composed of polypeptide members having modified loop regions in the CTLD from human or murine tetranectin. In certain embodiments, the polypeptide members can also have an N-terminal extension and/or a C-terminal extension of the CTLD. The N-terminal extension and/or C-terminal extension can provide effector function, enzyme function, further binding function, or multimerizing function. In one embodiment, at least one of the N-terminal extension and the C-terminal extension includes the non-CTLD-portions of a native C-type lectin-like protein or C-type lectin or a C-type lectin lacking a functional transmembrane domain. In one embodiment, the proteins are multimers of a moiety comprising the CTLD.

In other embodiments of the invention, the polypeptide members can have additional alterations in the loop regions, introduced by peptide grafting or identified by panning, that can provide effector function, enzyme function, further binding function, or multimerising function.

In other embodiments, the combinatorial library is composed of polypeptide members having modified loop regions in the CTLD region of a full-length human or murine tetranectin. In certain embodiments, the polypeptide members can have an N-terminal extension of the trimerization domain of tetranectin. The N-terminal extension can provide effector function, enzyme function, further binding function, or multimerizing function. In one embodiment, the N-terminal extension is a peptide or a polypeptide with known function or a peptide identified by panning.

In another aspect, the invention is directed to a library of nucleic acid molecules that encode any of the polypeptides described herein. In one embodiment, the invention provides a library of nucleic acid molecules encoding polypeptides having a CTLD with a randomized loop region, wherein the loop region of the CTLD is randomized according to any of the Schemes (a)-(i) described herein. In other embodiments, the invention provides a library of nucleic acid molecules encoding polypeptides having a CTLD randomized according to any of the Schemes (a)-(i) and having any of the further modifications or sequences described herein.

The library of nucleic acid molecules can be expressed in a display system having an observable phenotype that represents at least one property of the displayed expression products and the corresponding genotypes. Examples of suitable display systems include a phage display system; a yeast display system; a viral display system; a cell-based display system; a ribosome-linked display system; or a plasmid-linked display system.

In another aspect, the invention is directed to a method for generating a combinatorial library of any of the polypeptides described herein. Thus, the invention provides a method for generating a combinatorial library of polypeptides having a CTLD with a randomized loop region, wherein the loop region of the CTLD is randomized according to any of the Schemes (a)-(i) described herein. In one embodiment, the method comprises generating at least one random mutation in at least one of the four loops in the LSA region of the CTLD. In another embodiment, the method comprises generating at least one random mutation in at least one of the four loops in the LSA region and generating at least one random mutation in the loop in the LBA region of the CTLD. The random mutation can be created by oligonucleotide-directed randomization, DNA shuffling by random fragmentation, loop shuffling, loop walking, or error-prone PCR mutagenesis and other methods known in the art. In other embodiments, the invention provides a method for generating a combinatorial library of polypeptides having a CTLD randomized according to any of the Schemes (a)-(j) and having any of the further modifications or sequences described herein.

In another aspect, the invention is directed to a method for identifying and isolating a polypeptide having specific binding activity to a target molecule. In one embodiment, the method comprises providing a combinatorial library of polypeptides having a CTLD wherein the loop region of the CTLD is randomized according to any of the Schemes (a)-(j), contacting the combinatorial polypeptide library with the target molecule under conditions that allow for binding between a polypeptide and the target molecule; and isolating a polypeptide that binds to the target molecule. In another embodiment, the method comprises providing a combinatorial library of polypeptides having a CTLD randomized according to any of the Schemes (a)-(j) and any of the further modification or sequences described herein, contacting the combinatorial polypeptide library with the target molecule under conditions that allow for binding between a polypeptide and the target molecule; and isolating a polypeptide that binds to the target molecule. The method can further include a library of nucleic acid molecules encoding polypeptides of the combinatorial polypeptide library described herein, wherein the library of nucleic acids is expressed in a display system, wherein the display system comprises an observable phenotype that represents at least one property of the displayed expression products and the corresponding genotypes.

The invention is also directed to a method for the identification and isolation of a polypeptide that specifically binds to a target using a library of nucleic acid molecules. In one embodiment, the invention provides a method for the identification and isolation of a polypeptide capable of specifically binding to a target comprising the steps of: providing a library of nucleic acids encoding polypeptides having a CTLD with a randomized loop region, wherein the loop region of the CTLD is randomized according to any of Schemes (a)-(j), expressing the nucleic acid library in a display system to obtain an ensemble of polypeptides, in which the amino acid residues at one or more sequence positions differ between different members of said ensemble of polypeptides, contacting the ensemble of polypeptides with said target, and isolating a polypeptide that is capable of specifically binding to said target. In other embodiments, the method comprises providing a library of nucleic acid molecules encoding polypeptides having a CTLD randomized according to any of the Schemes (a)-(j) and having any of the further modifications or sequences described herein.

In another aspect, the invention provides a polypeptide having the scaffold structure of a C-type Lectin Like Domain (CTLD), wherein the polypeptide binds to a target other than a natural target for that CTLD and wherein the CTLD scaffold structure of the CTLD is modified according to any of the schemes (a)-(j). In one embodiment, the CTLD scaffold structure is modified according to any of the schemes (a)-(j) and further comprises any of the further modifications described herein, for example, modifications outside the CTLD loop region. In one embodiment, the polypeptide has the scaffold structure of the CTLD from human or mouse tetranectin and binds to a target other than plasminogen.

The polypeptide can be produced using a combinatorial library of polypeptides having a CTLD, wherein the loop region of the CTLD is randomized according to any of the Schemes (a)-(j), contacting the combinatorial polypeptide library with the target molecule under conditions that allow for binding between a polypeptide and the target molecule; and isolating a polypeptide that binds to the target molecule, wherein the target molecule is not the natural target for that CTLD. In one embodiment of this method, the CTLD is human or mouse tetranectin. In another embodiment of this method, the CTLD is randomized according to any of the Schemes (a)-(j) and comprises any of the further modifications described herein, for example, modifications outside the CTLD loop region.

### BRIEF DESCRIPTION OF THE FIGURES

FIG. 1 depicts an alignment of the amino acid sequences of ten CTLDs of known three-dimensional structure. The sequence locations of main secondary structural elements are indicated above each sequence and labeled in sequential numerical order wherein “αX” denotes an α-helix number X, and βY denotes a β-strand number Y. The four cysteine residues involved in the formation of the two conserved disulfide bridges of the CTLDs are indicated and numbered as C_{I}, C_{II}, C_{III}, and C_{IV}, where the disulfide bridges are formed by C_{I}-C_{IV} and C_{II}-C_{III}. The various loop regions in the human tetranectin sequence are indicated by underlining.

The various CTLDs include: “hTN” (human tetranectin, Nielsen et al., (1997)); “MBP” (mannose binding protein, Weis et al., (1991); Sheriff et al., (1994)); “SP-D” (surfactant protein D, Hakansson et al., (1999)); “LY49A” (NK receptor LY49A, Tormo et al., (1999)); “H1-ASR” (H1 subunit of the asialoglycoprotein receptor, Meier et al., (2000)); “MMR-4” (macrophage mannose receptor domain 4, Feinberg et al., (2000)); “IX-A” and “IX-B” (coagulation factors IX/X-binding protein domain A and B, respectively, Mizuno et al., (1997); “Lit” (lithostatine, Bertrand et al., (1996)); and “TU14” (tunicate C-type lectin, Poget et al., (1999)).

FIG. 2 depicts an alignment of the nucleotide and amino acid sequences of the coding regions of the mature forms of human and murine tetranectin with an indication of known secondary structural elements.

FIG. 3 depicts an alignment of several C-type lectin domains from tetranectins isolated from human (Swissprot P05452), mouse (Swissprot P43025), chicken (Swissprot Q9DDD4), bovine (Swissprot Q2KIS7), Atlantic salmon (Swissprot B5XCV4), frog (Swissprot Q5I0R9), zebrafish (GenBank XP_{—}701303), and related CTLD homologues isolated from cartilage of cattle (Swissprot u22298) and reef shark (Swissprot p26258).

FIG. 4 depicts the three dimensional structure (ribbon format) for human tetranectin, depicting the secondary structural features of the protein. The structure was solved in the Ca²⁺-bound form.

FIG. 5A depicts the three dimensional overlay structures of the CTLDs for human tetranectin (HTN) and several tetranectin homologues, including human mannose binding protein (MBP), rat mannose binding protein-C (MBP-C), human surfactant protein D, rat mannose binding protein-A (MBP-A), and rat surfactant protein A. The CTLD overlay structures were generated using Swiss PDB Viewer DeepView v. 4.0.1 for MacIntosh using the three-dimensional structure of human tetranectin as a template. FIG. 5B shows the corresponding amino acid sequences of the CTLDS for human tetranectin and the tetranectin homologues depicted in FIG. 5A. In Figure B, 1HUP=human mannose binding protein, 1BV4A=rat mannose binding protein, 2GGUA=human surfactant protein D, 1KXOA=rat mannose binding protein A, 1R13=rat surfactant protein A.

FIG. 6A depicts the three dimensional overlay structures of the CTLDs for human tetranectin (HTN) and several tetranectin homologues, including human pancreatitis-associated protein, human dendritic cell-specific ICAM-3-grabbing non-integrin 2 (DC-SIGNR), rat aggrecan, mouse scavenger receptor, and human scavenger receptor. The CTLD overlay structures were generated using Swiss PDB Viewer DeepView v. 4.0.1 for MacIntosh using the three-dimensional structure of human tetranectin as a template. FIG. 6B shows the corresponding amino acid sequences of the CTLDS for human tetranectin and the tetranectin homologues depicted in FIG. 6A. In FIG. 6B, 1TDQB=rat aggrecan, 1UV0A=human pancreatitis-associated protein, 2OX8A=human scavenger receptor, 2OX9A=mouse scavenger receptor, and 1 SL6A=human DC-SIGNR)

FIG. 7 shows the PCR strategy for creating randomized loops in a CTLD.

FIG. 8 shows the DNA and amino acid sequence of the human tetranectin CTLD modified to contain restriction sites for cloning, indicating the Ca2+ binding sites. Restriction sites are underscored with solid lines. Loops are underlined with dashed lines. Calcium coordinating residues are in bold italics and include Site 1: D116, E120, G147, E150, N151; Site 2: Q143, D145, E150, D165. The CTLD domain starts at amino acid A45 in bold (i.e. ALQTVCL . . . ). Changes to the native tetranectin (TNCTLD) base sequence are shown in lower case. The restriction sites were created using silent mutations that did not alter the native amino acid sequence.

FIG. 9 depicts a non-limiting strategy for lengthening and introducing randomization in a CTLD loop region.

FIG. 10 shows the results of experiments measuring cell death in the presence of five DR 5 ATRIMERs™: 4a8c, 2a1a, 1a7b, 9b3d and 8b6b. H2122 lung adenocarnoma cells and A2780 ovarian carcinoma cells were incubated at 1×10⁴ cells/well with DR 5 ATRIMERs™ (20 μg/mL) or TRAIL (0.2 μg/mL). Data are expressed as percent cell death relative to the respective buffer control.

FIG. 11 shows the results of an experiment comparing binding of the polypeptides of the invention and native human IL-23 to human IL-23R.

FIG. 12 shows the results of an experiment comparing IL-23-induced IL-17 production in the presence of ATRIMER™ complex 4G8 of the invention, native human IL-23, and Ustekinumab.

FIG. 13 shows the results of an experiment comparing IL-23 induced IL-17 production in the presence of ATRIMER™ complex 1A4 of the invention and Ustekinumab.

FIG. 14 shows the results of an experiment comparing IL-12-induced IFNγ production in the presence of ATRIMER™ complex 4G8 of the invention, native human IL-23, and Ustekinumab.

FIG. 15 shows the results of an experiment comparing Stat-3 phosphorylation in NKL cell in in response to IL-23 and the polypeptides of the invention.

FIGS. 16A and 16B are tables showing experimental results associated with several ATRIMER™ polypeptide complexes of the invention.

### DETAILED DESCRIPTION OF THE INVENTION

### Definitions

All scientific and technical terms used throughout the application should be understood to have their common scientific/technical meaning, unless specifically indicated otherwise. Similarly when the singular form of a term or article is used, it should be understood to also encompass the plural form of that term or article.

The terms “C-type lectin-like protein” and “C-type lectin” are used to refer to any protein or polypeptide present in or encoded in the genomes of any eukaryotic species, wherein the protein or polypeptide contains one or more C-type lectin domains (CTLDs) or one or more domains belonging to any subgroup of CTLD, (e.g., the CRDs, which can bind carbohydrate ligands). The definition includes membrane attached C-type lectin-like proteins and C-type lectins, “soluble” C-type lectin-like proteins and C-type lectins lacking a functional transmembrane domain and variant C-type lectin-like proteins and C-type lectins in which one or more amino acid residues have been altered in vivo by glycosylation or any other post-synthetic modification, as well as any product that is obtained by chemical and enzymatic modification of C-type lectin-like proteins and C-type lectins. In the claims and throughout the specification certain alterations can be defined with reference to particular amino acid residue numbers of a CTLD or a CTLD-containing protein. See, Essentials of Glycobiology, second edition. Edited by A. Varki, R. D. Cummings, J. D. Esko, H H. Freeze, P. Stanley, C. R. Bertozzi, G. W. Hart, M. E. Etzler. CHS Press.

The CTLD consists of roughly 120 amino acid residues and, characteristically, contains two or three intra-chain disulfide bridges. Although the similarity at the amino acid sequence level between CTLDs from different proteins is relatively low, the three dimensional structures of a number of CTLDs have been found to be highly conserved, with the structural variability essentially confined to the loop-region, often defined by up to five loops. Several CTLDs contain either one or two binding sites for calcium and most of the side chains which interact with calcium are located in the loop-region.

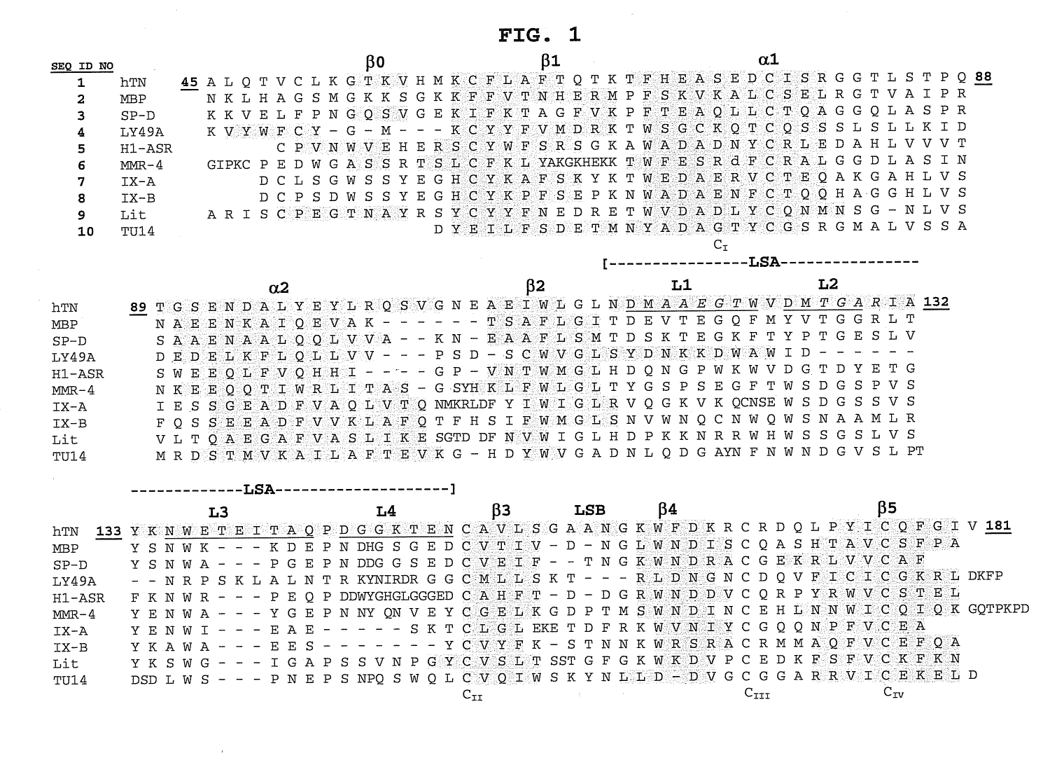

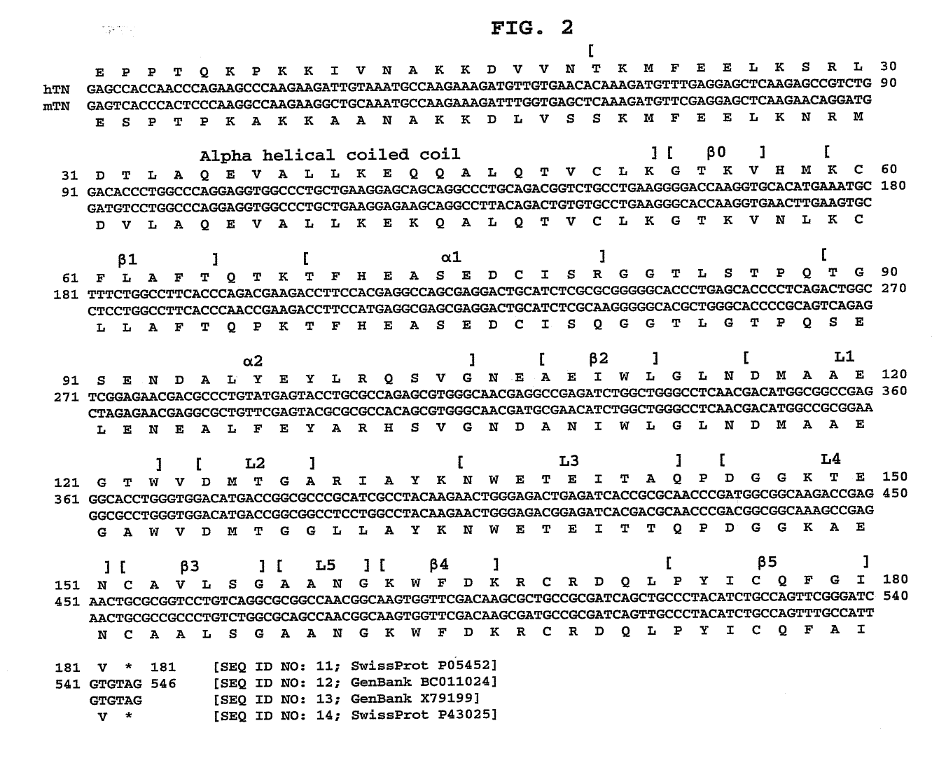

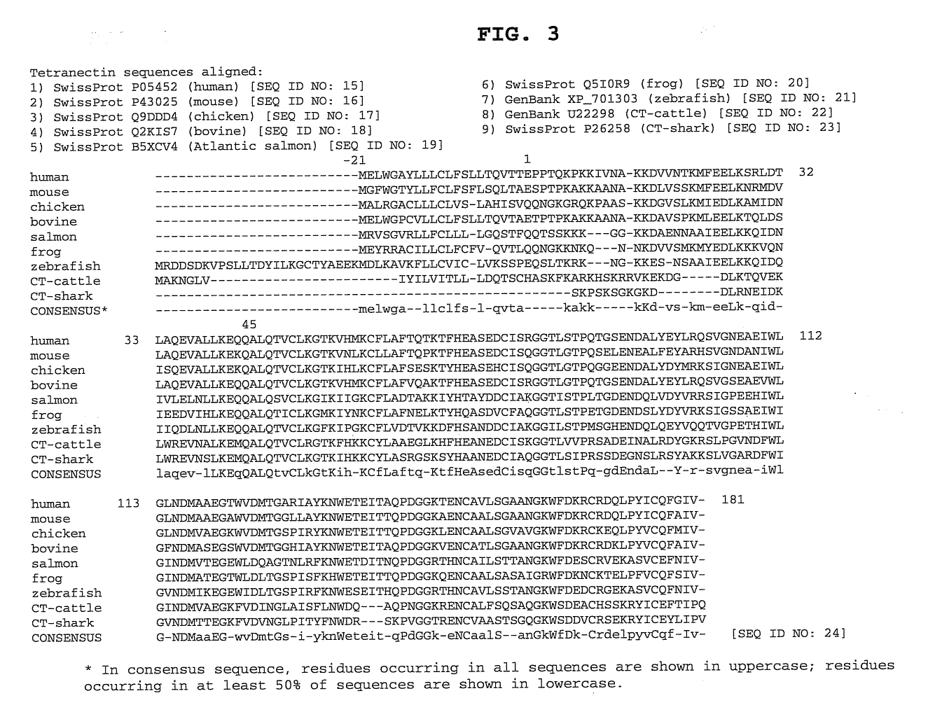

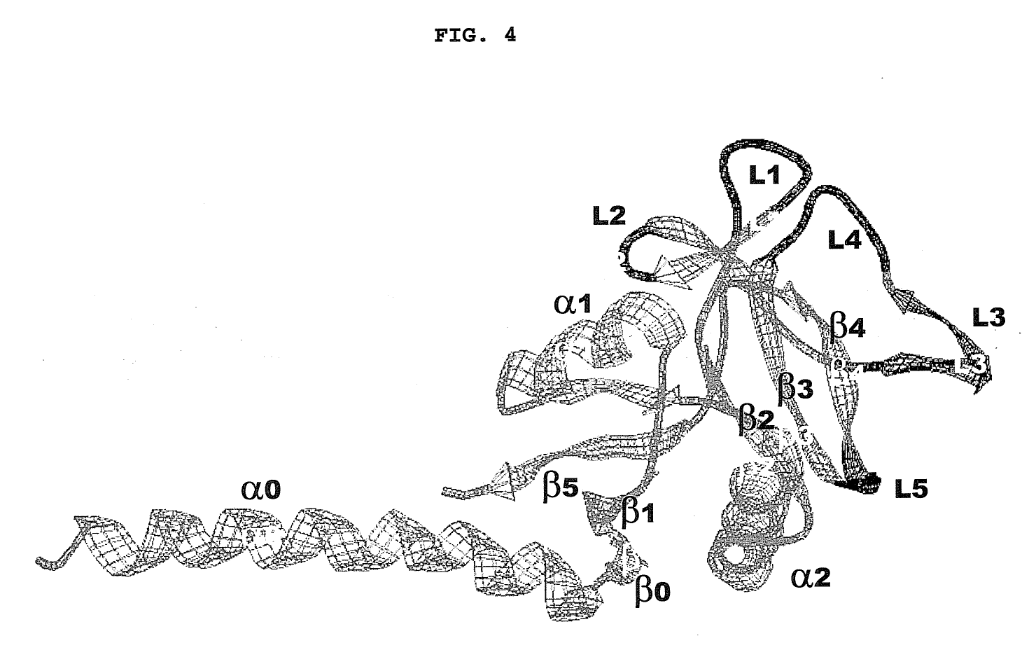

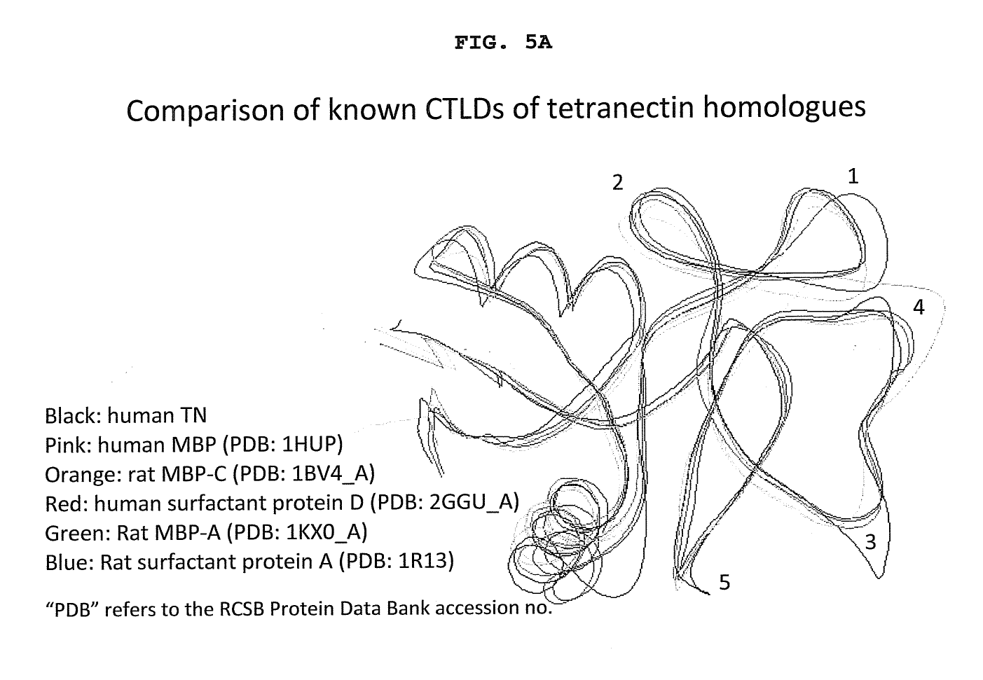

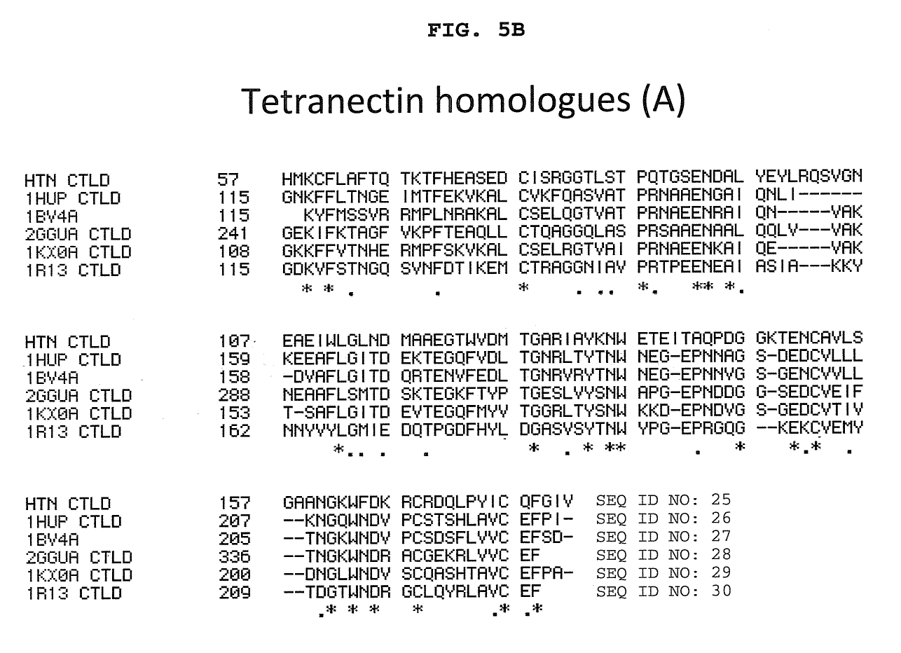

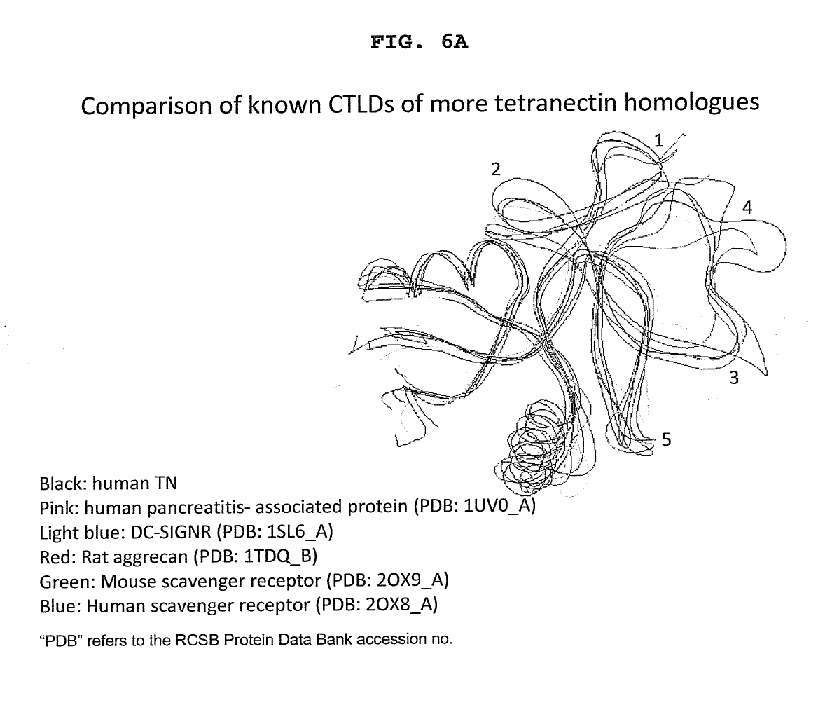

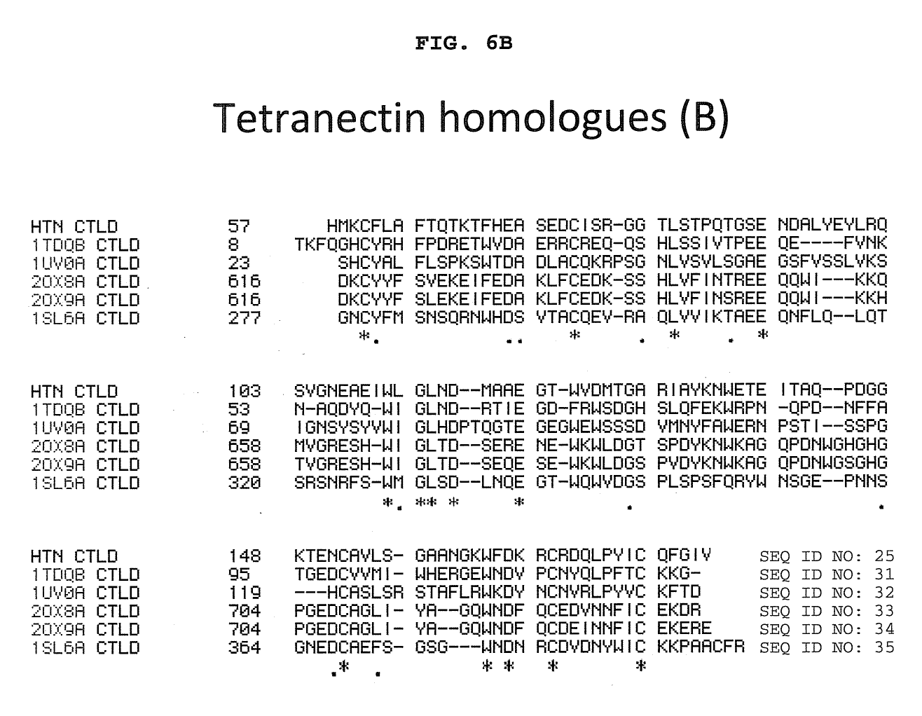

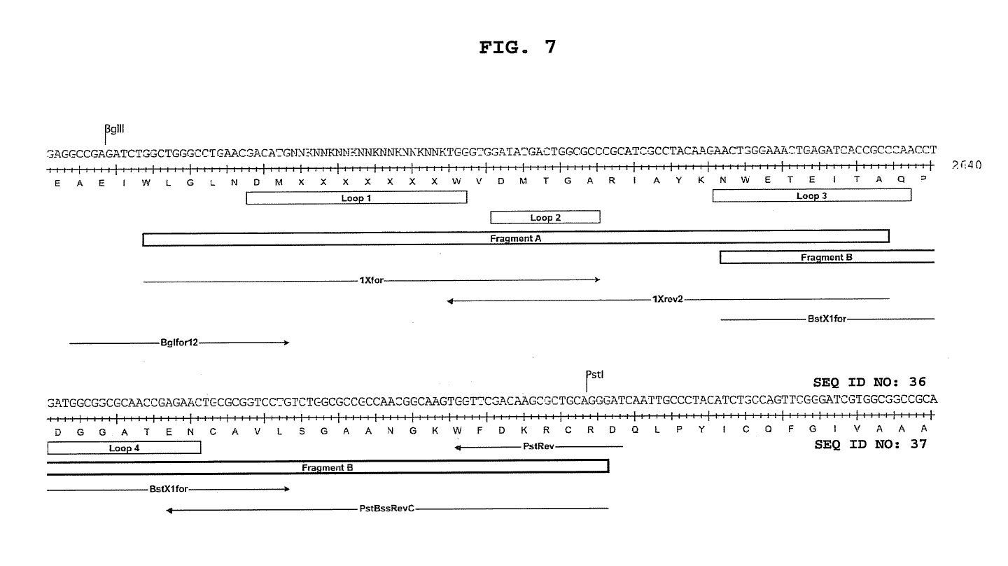

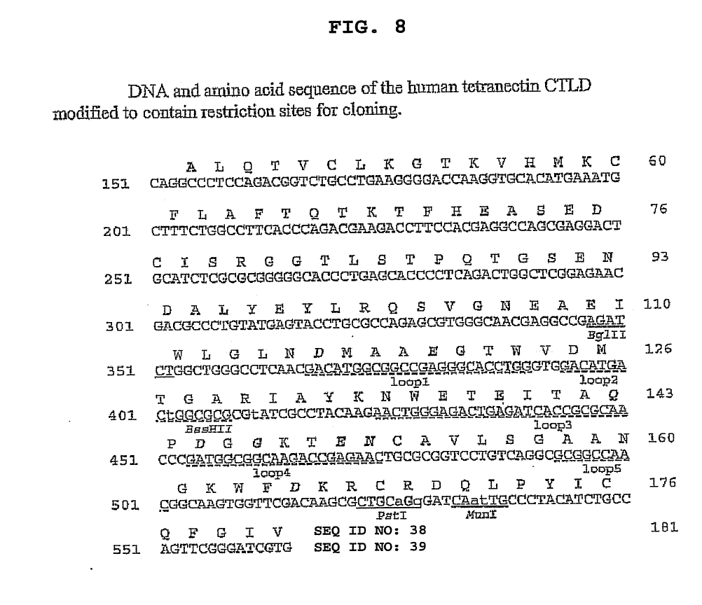

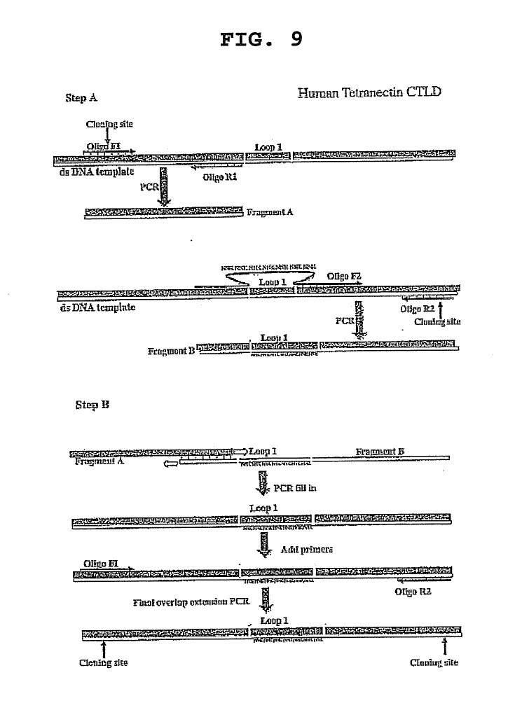

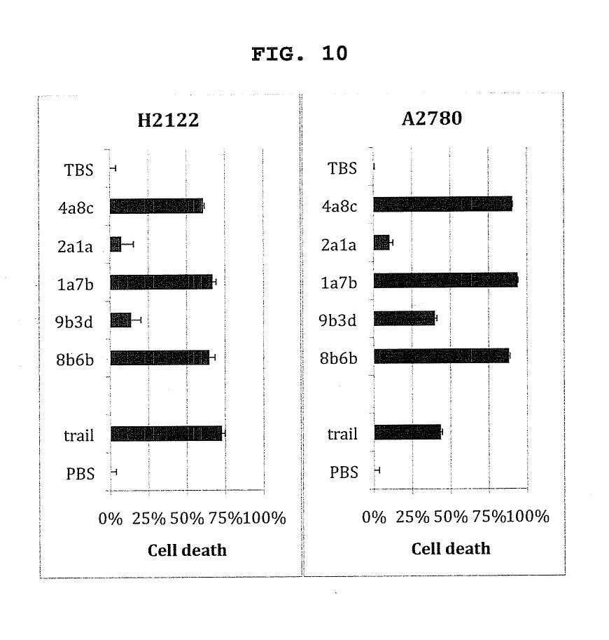

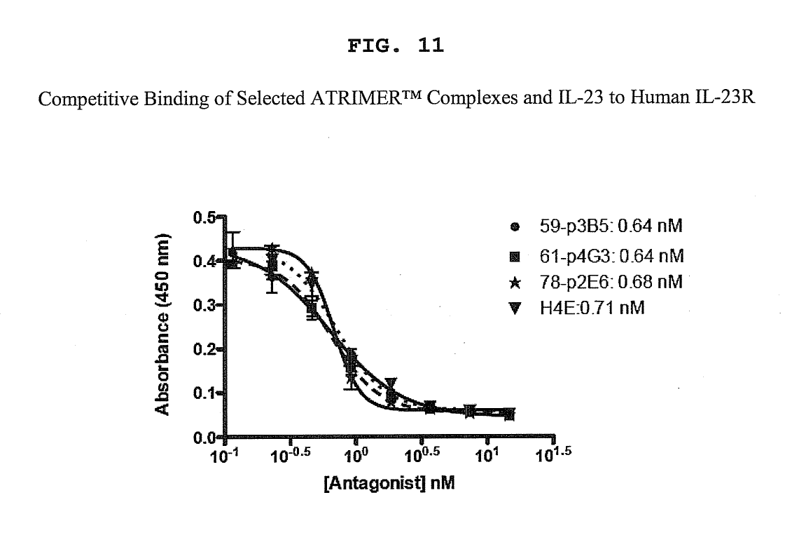

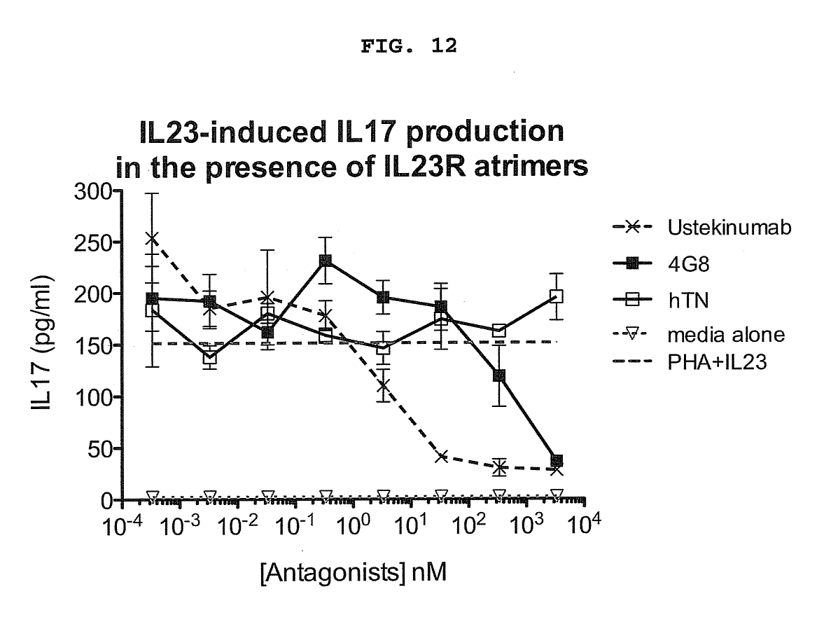

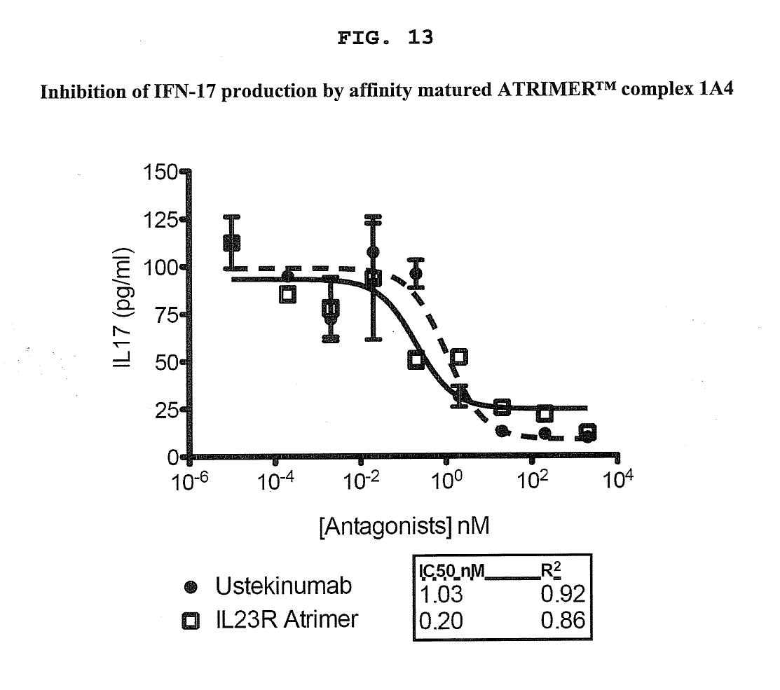

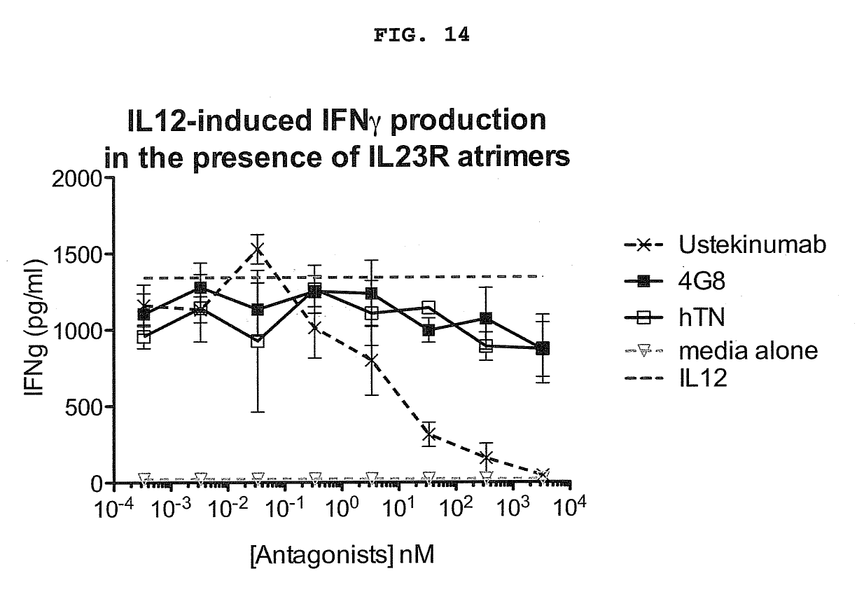

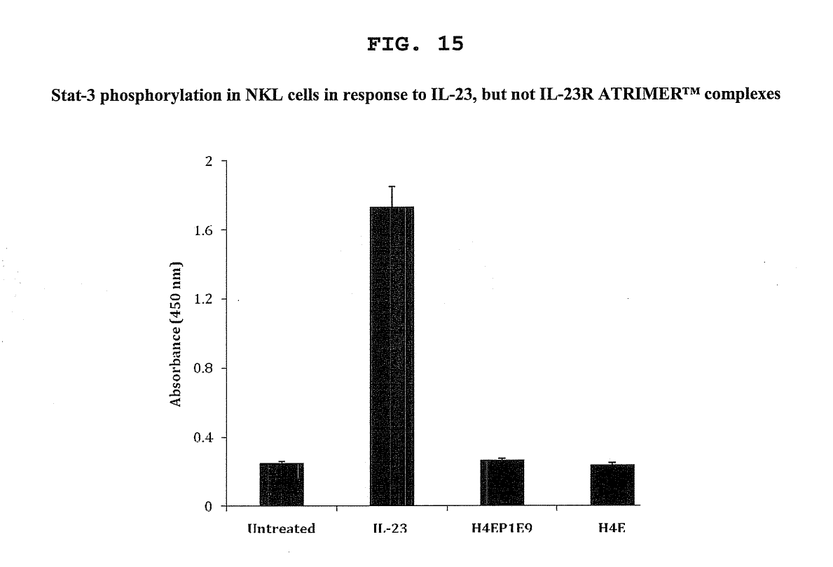

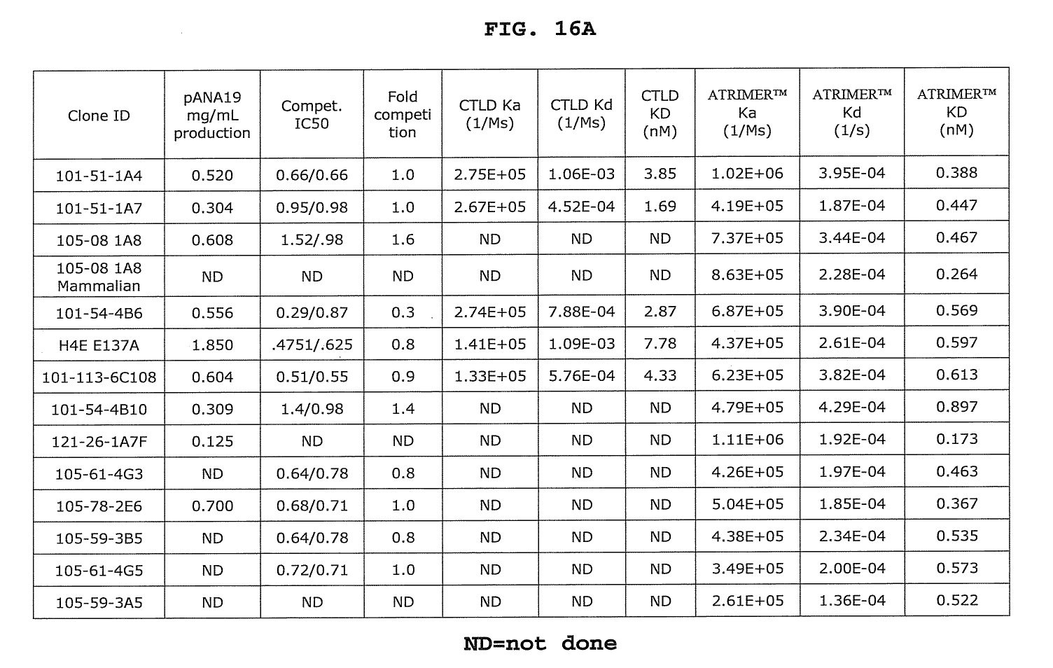

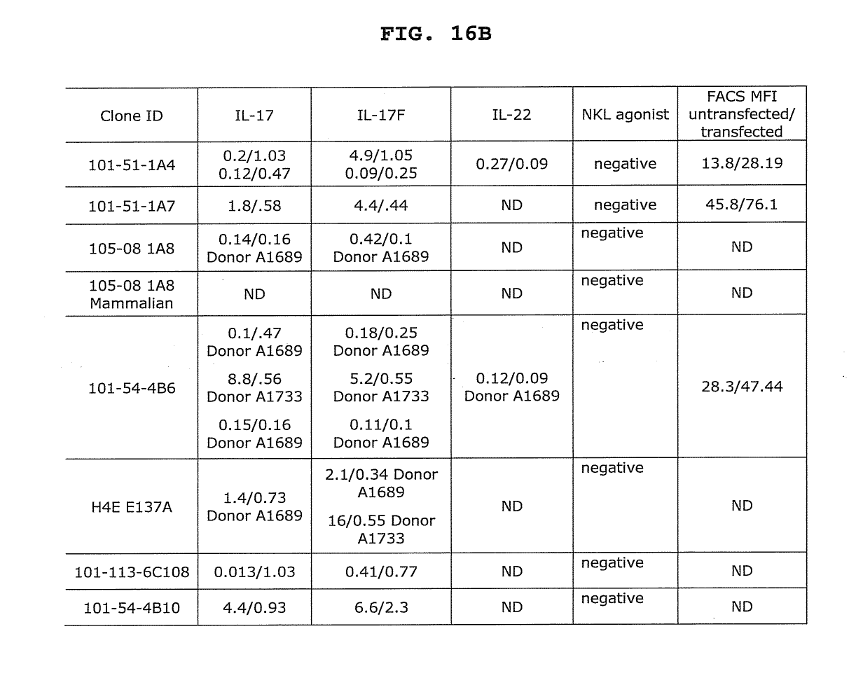

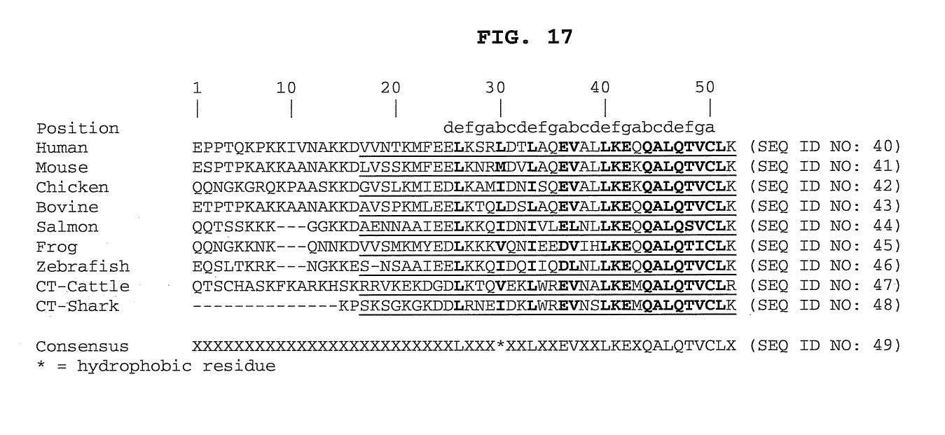

On the basis of CTLDs for which three dimensional structural information is available, it has been inferred that the canonical CTLD is structurally characterized by seven main secondary-structure elements (i.e. five β-strands and two α-helices) sequentially appearing in the order β1, α1, α2, β2, β3, β4, and β5. FIG. 1 illustrates an alignment of the CTLDs of known three dimensional structures of ten C-type lectins. In all CTLDs for which three dimensional structures have been determined, the β-strands are arranged in two anti-parallel β-sheets, one composed of β1 and β5, the other composed of β2, β3 and β4. An additional β-strand, β0, often precedes β1 in the sequence and, where present, forms an additional strand integrating with the β1, β5-sheet. Further, two disulfide bridges, one connecting α1 and β5 (C_{I}-C_{IV}) and one connecting β3 and the polypeptide segment connecting β4 and β5 (C_{II}-C_{III}) are invariantly found in all CTLDs characterized to date.

The conserved secondary structure elements (alpha helix and beta sheet) form a compact scaffold for a number of loops, which in the present context collectively are referred to as the “loop-region”, protruding out from the core. In the primary structure of the CTLDs, these loops are organized in two segments, loop segment A, LSA, and loop segment B, LSB. LSA represents the long polypeptide segment connecting β2 and β3 that often lacks regular secondary structure and contains up to four loops. LSB represents the polypeptide segment connecting the β-strands β3 and β4. Residues in LSA, together with single residues in β4, have been shown to specify the Ca²⁺- and ligand-binding sites of several CTLDs, including that of tetranectin. For example, mutagenesis studies, involving substitution of one or a few residues, have shown that changes in binding specificity, Ca²⁺-sensitivity and/or affinity can be accommodated by CTLD domains

As discussed herein, a number of proteins having CTLDs are known, including the following non-limiting examples: tetranectin, lithostatin, mouse macrophage galactose lectin, Kupffer cell receptor, chicken neurocan, perlucin, asialoglycoprotein receptor, cartilage proteoglycan core protein, IgE Fc receptor, pancreatitis-associated protein, mouse macrophage receptor, Natural Killer group, stem cell growth factor, factor IX/X binding protein, mannose binding protein, bovine conglutinin, bovine CL43, collectin liver 1, surfactant protein A, surfactant protein D, e-selectin, tunicate c-type lectin, CD94 NK receptor domain, LY49A NK receptor domain, chicken hepatic lectin, trout c-type lectin, HIV gp 120-binding c-type lectin, and dendritic cell immunoreceptor. See U.S. 2007/0275393, which is incorporated by reference herein in its entirety.

The terms “amino acid,” “amino acids,” and “amino acid residues” refer to all naturally occurring L-amino acids, as well as non-naturally occurring amino acids. This definition is meant to include norleucine, ornithine, and homocysteine. The naturally occurring L-amino acids can be classified according to the chemical composition and properties of their side chains. They are broadly classified into two groups, charged and uncharged. Each of these groups is divided into subgroups to classify the amino acids more accurately: A. Charged Amino Acids—(A.1. Acidic Residues): Asp, Glu; (A.2. Basic Residues): Lys, Arg, His, Orn; B. Uncharged Amino Acids—(B.1. Hydrophilic Residues): Ser, Thr, Asn, Gln; (B.2. Aliphatic Residues): Gly, Ala, Val, Leu, Ile, Nle; (B.3. Non-polar Residues): Cys, Met, Pro, Hcy; (B.4. Aromatic Residues): Phe, Tyr, Trp.

A “non-natural amino acid” or “non-naturally occurring amino acid” refers to an amino acid that is not one of the 20 common amino acids including, for example, amino acids that occur by modification (e.g. post-translational modifications) of a naturally encoded amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

Many of the unnatural amino acids suitable for use in the present invention are commercially available, e.g., from Sigma (USA) or Aldrich (Milwaukee, Wis., USA). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of .gamma.-Dipeptides of Glutamic Acid from Phthylated Intermediates. J. Chem. Soc., 3315-3319; Friedman, O. M. & Chatterrji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4[[4-(diethylamino)-1-methylbutyl]amino]quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates from L-Asparagine. Application to the Total Synthesis of (+)-Apovincamine through Amino Acid Decarbonylation and Iminium Ion Cyclization. J. Org. Chem. 1989: 1859-1866; Barton et al., (1987) Synthesis of Novel α-Amino-Acids and Derivatives Using Radical Chemistry: Synthesis of L- and D-α-Amino-Adipic Acids, L-α-aminopimelic Acid and Appropriate Unsaturated Derivatives. Tetrahedron Lett. 43: 4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35: 4602-7. See also, US 2004/0198637 and US 2005/0170404, each of which is incorporated by reference herein in their entirety.

The terms “amino acid modification(s)” and “modification(s)” refer to amino acid substitutions, deletions or insertions or any combinations thereof in an amino acid sequence relative to the native sequence. Substitutional variants herein are those that have at least one amino acid residue in a native CTLD sequence removed and a different amino acid inserted in its place at the same position. The substitutions may be single, where only one amino acid in the molecule has been substituted, or they may be multiple, where two or more amino acids have been substituted in the same molecule. Specific reference to more than one amino acid substitution in a CTLD refers to multiple substitutions in which each individual amino acid substitution can occur at any amino acid position within the CTLD, including consecutive and non-consecutive amino acid positions. Likewise, specific reference to more than one amino acid insertion or deletion in a CTLD refers to multiple insertions or deletions in which each individual amino acid insertion or deletion can occur at any amino acid position within the CTLD, including consecutive and non-consecutive amino acid positions.

The terms “nucleic acid molecule encoding”, “DNA sequence encoding”, and “DNA encoding” refer to the order or sequence of deoxyribonucleotides along a strand of deoxyribonucleic acid. The order of these deoxyribonucleotides determines the order of amino acids along the polypeptide chain. The DNA sequence thus encodes the amino acid sequence.

The terms “randomize,” “randomizing” and “randomized” as well as any similar terms used in any context to identify randomized polypeptide or nucleic acid sequences, refer to ensembles of polypeptide or nucleic acid sequences or segments, in which the amino acid residue or nucleotide at one or more sequence positions may differ between different members of the ensemble of polypeptides or nucleic acids, such that the amino acid residue or nucleotide occurring at each such sequence position may belong to a set of amino acid residues or nucleotides that may include all possible amino acid residues or nucleotides or any restricted subset thereof. The terms are often used to refer to ensembles in which the number of possible amino acid residues or nucleotides is the same for each member of the ensemble, but may also be used to refer to such ensembles in which the number of possible amino acid residues or nucleotides in each member of the ensemble may be any integer number within an appropriate range of integer numbers.

The terms “modulate” or “modulating” when used with reference to either the binding affinity of a CTLD to plasminogen, metal (e.g., Mg²⁺, Ca²⁺, Zn²⁺, Mn²⁺, etc.) or any other target molecule refer to a change in the binding affinity of a modified CTLD polypeptide to either plasminogen or metal ion or target molecule relative to the binding affinity of the native (unmodified) CTLD polypeptide. Thus, “modulating” includes increasing binding affinity, decreasing binding affinity, and/or abolishing or abrogating binding affinity (although not to the exclusion of the specific recitation of the terms “abolishing” or “abrogating” plasminogen, metal ion, or target molecule binding activity).

When referring to a binding pair, such as ligand/receptor, antibody/antigen, or other binding pair, binding is measured in a binding reaction which is determinative of the presence of a member of a binding pair in a heterogeneous population of another member of the binding pair. Under designated conditions, “specific binding” occurs when one member of the binding pair binds to another member of the binding pair in a heterologous population and does not bind in a significant amount to other proteins or polypeptides present in the sample. Specific binding can be measured using the methods described herein, including Biacore and ELISA.

The term “1X-2 Library” refers to a combinatorial polypeptide library comprising polypeptide members that have a C-type lectin domain (CTLD) comprising amino acid modifications in at least one of the four loops in the LSA of the CTLD, wherein the amino acid modifications comprise at least two amino acid insertions in Loop 1 and random substitution of at least five amino acids within Loop 1 of the CTLD.

The term “1-2 library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least five amino acids within Loop 1 and random substitution of at least three amino acids within Loop 2.

The term “1-4 library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least seven amino acids within Loop 1, at least three amino acid insertions in Loop 4, and random substitution of at least two amino acids.

The term “3X library” refers to a combinatorial polypeptide library comprising polypeptide members that have a C-type lectin domain (CTLD) comprising amino acid modifications in at least one of the four loops in the LSA of the CTLD, wherein the amino acid modifications comprise a mixture of random substitution of at least six amino acids, random substitution of at least six amino acids and at least one amino acid substitution, and random substitution of at least six amino acids and at least two amino acid substitutions in Loop 3.

The term “3-4X library” refers to a combinatorial polypeptide library comprising polypeptide members that have a C-type lectin domain (CTLD) comprising amino acid modifications in at least one of the four loops in the LSA of the CTLD, wherein the amino acid modifications comprise at least three amino acid insertions in Loop 3 and random substitution of at least three amino acids within Loop 3 and comprise at least three amino acid insertions in Loop 4 and random substitution of at least three amino acids within Loop 4.

The term “3-4 combo library” refers to a combinatorial polypeptide library comprising polypeptide members that have a C-type lectin domain (CTLD) comprising amino acid modifications in at least one of the four loops in the LSA of the CTLD, wherein the amino acid modifications comprise a modification that combines two loops into a single loop, wherein the two combined loops are Loop 3 and Loop 4.

The term “4 library” refers to a combinatorial polypeptide library comprising polypeptide members that have a C-type lectin domain (CTLD) comprising amino acid modifications in at least one of the four loops in the LSA of the CTLD, wherein the amino acid modifications comprise at least four amino acid insertions in Loop 4 and random substitution of at least three amino acids within Loop 4.

The term “3-5 library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least five amino acids within Loop 3 and random substitution of at least three amino acids within Loop 5.

The term “Loop 3X loop library” refers to a combinatorial polypeptide library comprising polypeptide members that have a C-type lectin domain (CTLD) comprising amino acid modifications in at least one of the four loops in the LSA of the CTLD, wherein the amino acid modifications comprise random substitution of at least one amino acid and at least six amino acid insertions.

Combinatorial Polypeptide Libraries with Modified CTLD

The invention relates generally to a combinatorial polypeptide library comprising polypeptide members having a C-type lectin domain (CTLD) with a randomized loop region, in which the randomized loop region has been modified from the native sequence of the CTLD. The randomized loop region of the CTLD can comprise one or more amino acid modifications in at least one of the four loops in the loop segment A (LSA) of the CTLD and can further comprise one or more amino acid modifications in the loop in Loop Segment B (LSB) (also known as loop 5). The invention also relates to methods for generating and using the randomized combinatorial polypeptide libraries. By applying standard combinatorial methods known in the chemical, recombinant protein and antibody arts, the libraries and methods of the invention allow for the generation, screening, and identification of protein products that exhibit binding specificity to target molecules of interest.

The variation of binding site configuration among naturally occurring CTLDs shows that their common core structure can accommodate many essentially different configurations of the ligand binding site (see, e.g., US 2007/0275393). CTLDs are therefore particularly well suited to serve as a basis for constructing such new and useful protein products with desired binding properties. Accordingly, while in one aspect the invention relates to combinatorial polypeptide libraries comprising modifications to the loop region of the CTLD (LSA and LSB), other modifications to the general CTLD core structure (i.e., the β-strands and α-helices) can be made without affecting the utility of the libraries described herein. One of skill in the art can target particular modifications in the CTLD core structure that will retain CTLD functionality. For example, based on secondary and tertiary structures of various polypeptides comprising CTLDs, hydropathy, charge (ionic), and hydrogen bonding interactions can all be taken into consideration, and appropriate substitutions made which retain CTLD function. Such modifications include conservative amino acid substitutions. In embodiments that comprise variants, such as deletion, insertion, or substitution variants in the region outside of the loop region of the CTLD, the percent identity can be as low as 50%. In other embodiments comprising such variation within the CTLD region, variants are at least 80% identical to any given CTLD sequence, or CTLD consensus sequence. In certain embodiments such variants are at least 85%, 86%, 87%, 88%, 89%, 90%, 91%, 92%, 93%, 94%, 95%, 96%, 97%, 98%, or 99%, identical to any CTLD sequence, or CTLD consensus sequence.

The CTLD used in the combinatorial libraries can be derived from any CTLD. Examples of suitable CTLDs are CTLDs described herein (i.e., FIGS. 1-3) and in US 2007/0275393, which is incorporated by reference herein in its entirety (i.e., FIG. 1 and Table 1) and CTLDs otherwise known in the art. In certain embodiments, the CTLD has the following secondary structure: five β-strands and two α-helices sequentially appearing in the order β1, α1, α2, β2, β3, β4, and β5, the β-strands being arranged in two anti-parallel β-sheets, one composed of β1 and β5, the other composed of β2, β3 and β4, at least two disulfide bridges, one connecting α1 and β5 and one connecting β3 and the polypeptide segment connecting β4 and β5, and a loop region containing loop segment A (LSA) and loop segment B (LSB) in which LSA connects β2 and β3, and LSB connects β3 and β4.

In particular embodiments, the CTLD sequence is a human or murine tetranectin CTLD sequence that is modified according to the invention. FIG. 2 shows the alignment of the nucleic acid and polypeptide sequences of human and mouse tetranectin CTLDs. In other embodiments, the CTLD is from a variety of peptides, for example, those shown in FIG. 3, which shows an alignment of several CTLDs from tetranectins isolated from human (Swissprot P05452), mouse (Swissprot P43025), chicken (Swissprot Q9DDD4), bovine (Swissprot Q2KIS7), Atlantic salmon (Swissprot B5XCV4), frog (Swissprot Q5I0R9), zebrafish (GenBank XP_{—}701303), and related CTLD homologues isolated from cartilage of cattle (Swissprot u22298) and reef shark (Swissprot p26258).

Thus, in a broad aspect, the invention provides a polypeptide library comprising polypeptide members that comprise a C-type lectin domain (CTLD), wherein the CTLD comprises one or more amino acid modifications in at least one of the four loops in the loop segment A (LSA) of the CTLD, and/or in the loop in loop segment B (LSB) (Loop 5). Examples of polypeptide libraries comprising polypeptides having a C-type lectin domain comprising one or more amino acid modifications in at least one of the five loops in the loop region (LSA and LSB) of the CTLD are described herein.

In certain embodiments of the polypeptide libraries, the polypeptide members have CTLDs in which one, two, three, four, or five of the CTLD loops have one or more amino acid modifications, wherein the one or more modifications include at least one amino acid insertion that extends the loop region beyond its original length. In certain of these embodiments, the one or more modifications include from 1 to about 30 amino acid insertions (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid insertions) in any single loop in the loop region (LSA and LSB). In certain of these embodiments, the one or more modifications include at least one amino acid insertion in at least two of the five loops in the loop region (e.g., two, three, or four loops in LSA or one, two, or three loops in LSA and one loop in LSB).

In certain embodiments, the polypeptide libraries comprise polypeptide members that comprise a C-type lectin domain (CTLD), wherein the CTLD comprises one or more amino acid modifications in at least one of the five loops in the loop region (LSA and LSB), wherein certain Ca²⁺ coordinating amino acids in the loop regions are retained. In other embodiments, the polypeptide libraries comprise polypeptide members that comprise a C-type lectin domain (CTLD), wherein the CTLD comprises one or more amino acid modifications in at least one of the five loops in the loop region (LSA and LSB), wherein certain amino acid(s) involved with plasminogen binding activity are eliminated.

In certain embodiments of this aspect, the polypeptide library comprises polypeptide members that comprise a C-type lectin domain (CTLD), wherein the CTLD comprises one or more amino acid modifications in regions of the CTLD that fall outside of the LSA and LSB regions. Accordingly, such modifications can be designed or randomly generated in any one or more of the beta strand and/or alpha helical regions. An example of this is shown in Table 17.

The loop region of any CTLD, if not already identified or characterized, can be identified by using any variety of structural or sequence-based analysis using the existing sequence based information for any single structurally characterized CTLD or any combination of structurally characterized CTLDs. Typically, the loop regions are stretches of amino acids found between more ordered regions of the CTLD amino acid sequence (e.g., between the α-helices or β-strands), and typically have a more flexible conformation. Loop segment A (LSA) in a CTLD typically falls between the β2 and β3 strands of the canonical CTLD motif. The (LSA) contains smaller loop regions (loops 1, 2, 3, and 4), which are usually located between small beta sheet structures that provide a degree of order to the (LSA) (see, e.g., FIG. 4). CTLDs typically have a smaller loop structure (loop segment B, “LSB” or “loop 5”) located between β3 and β4.

As mentioned, the loop region of any CTLD can be identified using structural and/or sequence-based analyses based on the existing sequence information for any single structurally characterized CTLD or any combination of structurally characterized CTLDs. For example, the location of the loop region of any uncharacterized CTLD can be identified by aligning a prospective CTLD sequence with the group of structure-characterized CTLDs presented in FIG. 1. The sequence alignments shown in FIG. 1 were strictly elucidated from actual three dimensional structure data. Given that the polypeptide segments of corresponding structural elements of the framework also exhibit strong amino acid sequence similarities, FIG. 1 provides a set of direct sequence-structure signatures, which can readily be inferred from the sequence alignment. As shown in FIG. 1, the loop region (LSA and LSB) is flanked by segments corresponding to the β2-, β3-, and β4-strands (loops 1-4 of LSA typically fall between the β2 and β3 strands of the canonical CTLD and loop 5 of LSB is typically located between β3 and (34 of the CTLD). The β2-, β3-, and β4-strands can be identified by identification of their respective consensus sequences (published in US Patent Application Publication 2007/0275393). The loop region of the prospective CTLD can be identified by aligning the sequence of the prospective CTLD with the sequence shown in FIG. 1 and assigning approximate locations of framework structural elements as guided by the sequence alignment, i.e., identifying the β2-, β3-, and β4-strands, adjusting the alignment to ensure precise alignment of the four canonical cysteine residues involved in the formation of the two conserved disulfide bridges (C_{I}-C_{IV} and C_{II}-C_{III}, in FIG. 1) invariably found in all CTLDs characterized thus far. Furthermore, the loop regions of a prospective CTLD can be identified using known protein structure modeling programs, such as Swiss PDB Viewer DeepView v. 4.0.1 for MacIntosh, by aligning the sequence of prospective CTLD with any of the CTLD sequences in FIG. 1. Other protein modeling programs that can be used in the same manner are known in the art and available for public use, for example, MODELLER and Selvita SPMP 2.0 (See Sali A, Blundell T L. (1993) Comparative protein modelling by satisfaction of spatial restraints. J. Mol. Biol. 234, 779-815; Marti-Renom M A, Stuart A, Fiser A, Sánchez R, Melo F, Sali A. (2000) Comparative protein structure modeling of genes and genomes. Annu. Rev. Biophys. Biomol. Struct. 29, 291-325; Fiser A, Sali A. (2003) Modeller: generation and refinement of homology-based protein structure models. Methods Enzymol. 374:461-91).

The sequence-structure analyses also demonstrate that CTLDs can be used as frameworks in the construction of new classes of CTLD libraries. The additional steps involved in preparing starting materials for the construction of a new class of CTLD library on the basis of a CTLD for which the precise three dimensional structure has not yet been determined includes the following: (1) alignment of the sequence of the new CTLD with the sequence shown in FIG. 1; and (2) assignment of approximate locations of framework structural elements as guided by the sequence alignment, observing any requirement for minor adjustment of the alignment to ensure precise alignment of the four canonical cysteine residues involved in the formation of the two conserved disulfide bridges (C_{I}-C_{IV} and C_{II}-C_{III}, in FIG. 1).

The polypeptides comprising a CTLD used in the polypeptide libraries of the invention can be full-length proteins or partial proteins having a CTLD, for example, the full-length amino acid sequence or partial amino acid sequence of any of the proteins described herein and otherwise known. Alternatively, the polypeptides comprising a CTLD used in the polypeptide libraries of the invention can be polypeptides comprising only CTLD sequence, for example, the amino acid sequence of any of the CTLDs described herein and otherwise known. The polypeptides comprising CTLD sequence can have additional flanking C-terminal and/or N-terminal (non-CTLD) amino acid sequence.

In one aspect, the invention provides a combinatorial peptide library, and a library of nucleic acid sequences encoding the polypeptides of the library, wherein the CTLDs of the polypeptides have been modified according to a number of schemes, which have been labeled for the purposes of identification only as Schemes (a)-(j). While each scheme is more particularly described herein, the modifications are at least as follows:

amino acid modifications in at least one of four loops in loop segment A (LSA) of the CTLD, wherein the amino acid modifications comprise an insertion of at least one amino acid in Loop 1 and random substitution of at least five amino acids within Loop 1;

amino acid modifications in at least one of four loops in loop segment A (LSA) of the CTLD, wherein the amino acid modifications comprise random substitution of at least five amino acids within Loop 1 and random substitution of at least three amino acids within Loop 2;

amino acid modifications in at least one of four loops in loop segment A (LSA) of the CTLD, wherein the amino acid modifications comprise random substitution of at least seven amino acids within Loop 1 and at least one amino acid insertion in Loop 4;

amino acid modifications in at least one of four loops in loop segment A (LSA) of the CTLD, wherein the amino acid modifications comprise at least one amino acid insertion in Loop 3 and random substitution of at least three amino acids within Loop 3;

amino acid modifications in at least one of four loops in loop segment A (LSA) of the CTLD, wherein the amino acid modifications comprise a modification that combines two loops into a single loop, wherein the two combined loops are Loop 3 and Loop 4;

amino acid modifications in at least one of four loops in loop segment A (LSA) of the CTLD, wherein the amino acid modifications comprise at least one amino acid insertion in Loop 4 and random substitution of at least three amino acids within Loop 4;

amino acid modifications in at least one of the five loops in loop segment A (LSA) and loop segment B (LSB) of the CTLD, wherein the amino acid modifications comprise random substitution of at least five amino acid residues in Loop 3 and random substitution of at least three amino acids within Loop 5;

amino acid modifications in at least one of the four loops in loop segment A (LSA) of the CTLD, wherein the amino acid modifications comprise random substitution of at least one amino acid and insertion of at least six amino acids in Loop 3;

(i) amino acid modifications in at least one of the four loops in the loop segment A (LSA) of the CTLD, wherein the amino acid modifications comprise a mixture of (1) random substitution of at least six amino acids in Loop 3 and (2) random substitution of at least six amino acids and at least one amino acid insertion in Loop 3; and

(j) amino acid modifications in at least one of the four loops in the loop segment A (LSA) of the CTLD, wherein the amino acid modifications comprise at least four or more amino acid insertions in at least one of the four loops in the loop segment A (LSA) or loop 5 in loop segment B (LSB) of the CTLD.

With respect to scheme (a), the invention provides a combinatorial polypeptide library comprising polypeptide members having a randomized C-type lectin domain (CTLD), wherein the randomized CTLD includes amino acid modifications in at least one of the four loops in LSA or in the loop in LSB of the CTLD, wherein the amino acid modifications comprise at least one amino acid insertion in Loop 1 and random substitution of at least five amino acids within Loop 1.

In certain embodiments of this aspect of the combinatorial library, when the CTLD is from human tetranectin, the CTLD also has a random substitution of Arginine-130. For CTLDs other than the CTLD of human tetranectin, this peptide is located immediately adjacent to the C-terminal peptide of Loop 2 in the C-terminal direction. For example, in mouse tetranectin, this peptide is Gly-130. In certain embodiments of this aspect of the combinatorial library, when the CTLD is from tetranectin, for example human or mouse tetranectin, the CTLD includes a substitution of Lysine-148 to Alanine in Loop 4.

In certain embodiments, when the combinatorial library has the modified CTLD of Scheme (a), the amino acid modifications comprise two amino acid insertions in Loop 1 and random substitution of at least five amino acids within Loop 1. In other embodiments, when the combinatorial library has the modified CTLD of scheme (a) and the CTLD is from human tetranectin, the amino acid modifications comprise at least one amino acid insertion in Loop 1, random substitution of at least five amino acids within Loop 1, and include a random substitution of Arginine 130. In one specific embodiment, when the combinatorial library has the modified CTLD of scheme (a) and the CTLD is from human tetranectin, the amino acid modifications comprise two amino acid insertions in Loop 1, random substitution of five amino acids within Loop 1, and a random substitution of Arginine 130. In one specific embodiment, when the combinatorial library has the modified CTLD of scheme (a) and the CTLD is from mouse tetranectin, the amino acid modifications comprise two amino acid insertions in Loop 1, random substitution of five amino acids within Loop 1, and a random substitution of Leucine 130. In any of the embodiments for scheme (a), the amino acid modifications can further comprise a substitution of Lysine-148 to Alanine. Thus, in one specific embodiment of this aspect of the combinatorial library, the CTLD comprises two amino acid insertions in Loop 1, random substitution of at least five amino acids within Loop 1, random substitution of Arginine-130 or other amino acid located outside and adjacent to loop 2 in the C-terminal direction, and a substitution of lysine-148 to alanine in Loop 4.

With respect to scheme (b), the invention provides a combinatorial polypeptide library comprising polypeptide members having a randomized C-type lectin domain (CTLD), wherein the randomized CTLD comprises amino acid modifications in at least one of the four loops in the LSA of the CTLD, wherein the amino acid modifications comprise random substitution of at least five amino acids within Loop 1 and random substitution of at least three amino acids within Loop 2.

In certain embodiments of this aspect of the combinatorial library of scheme (b), when the CTLD is from tetranectin, the amino acid modifications comprise random substitution of at least five amino acids within Loop 1, random substitution of at least three amino acids within Loop 2, and random substitution of Arginine-130, or other amino acid located outside and adjacent to loop 2 in the C-terminal direction. In certain embodiments, when the combinatorial library has the modified CTLD of Scheme (b) and the CTLD is from human tetranectin, the amino acid modifications include random substitutions of at least five amino acids in Loop 1, random substitution of at least three amino acids in Loop 2, and include a random substitution of Arginine 130. In one embodiment, when the combinatorial library has the modified CTLD of Scheme (b) and the CTLD is from human tetranectin, the amino acid modifications include random substitutions of five amino acids in Loop 1, random substitution of three amino acids in Loop 2, and a random substitution of Arginine 130. In certain other embodiments, when the combinatorial library has the modified CTLD of Scheme (b) and the CTLD is from mouse tetranectin, the amino acid modifications include random substitutions of at least five amino acids in Loop 1, random substitution of at least three amino acids in Loop 2, and include a random substitution of Leucine 130. In one embodiment, when the combinatorial library has the modified CTLD of Scheme (b) and the CTLD is from mouse tetranectin, the amino acid modifications include random substitutions of five amino acids in Loop 1, random substitution of three amino acids in Loop 2, and a random substitution of Leucine 130. In any of the embodiments for scheme (b), the amino acid modifications can further comprise a substitution of Lysine-148 to Alanine. Thus, in one specific embodiment, the amino acid modifications comprise random substitution of at least five amino acids within Loop 1, random substitution of at least three amino acids within Loop 2, and random substitution of Arginine-130, or other amino acid located outside and adjacent to loop 2 in the C-terminal direction and a substitution of Lysine-148 to Alanine in Loop 4.

With respect to scheme (c), the invention provides a combinatorial polypeptide library comprising polypeptide members that have a randomized C-type lectin domain (CTLD), wherein the randomized CTLD comprises amino acid modifications in at least one of the four loops in loop segment A (LSA) of the CTLD, wherein the amino acid modifications comprise random substitution of at least seven amino acids within Loop 1 and at least one amino acid insertion in Loop 4.

In certain embodiments of this aspect of the combinatorial library, the polypeptide members of the combinatorial library further comprise random substitution of at least two amino acids within Loop 4. In certain other embodiments of this aspect, the amino acid modifications comprise three amino acid insertions within Loop 4 and optionally further comprise random substitution of at least two amino acids. In one embodiment, the amino acid modifications comprise random substitution of at least seven amino acids within Loop 1, at least three amino acid insertions in Loop 4, and random substitution of at least two amino acids within Loop 4. In one specific embodiment, the amino acid modifications comprise random substitution of seven amino acids within Loop 1, three amino acid insertions in Loop 4, and random substitution of two amino acids within Loop 4.

With respect to scheme (d), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise at least one amino acid insertion in loop 3 and random substitution of at least three amino acids within Loop 3.

In certain embodiments, when the combinatorial library has the modified CTLD of Scheme (d), the amino acid modifications can further comprise at least one amino acid insertion in Loop 4, and can further comprise random substitution of at least three amino acids within Loop 4. In any of the described embodiments for scheme (d), the amino acid modifications can comprise three amino acid insertions in Loop 3. In any of the described embodiments for scheme (d), the amino acid modifications can comprise three amino acid insertions in Loop 4. Thus, in certain embodiments, the amino acid modifications comprise random substitution of at least three amino acids within Loop 3, random substitution of at least three amino acids within Loop 4, at least one amino acid insertion in Loop 3 and at least one amino acid insertion in Loop 4. In certain embodiments, the amino acid modifications comprise random substitution of at least three amino acids within Loop 3, random substitution of at least three amino acids within Loop 4, at least three amino acid insertions in Loop 3 and at least three amino acid insertions in Loop 4. In one specific embodiment, the amino acid modifications comprise random substitution of three amino acids within Loop 3, random substitution of three amino acids within Loop 4, three amino acid insertions in Loop 3, and three amino acid insertions in Loop 4. In any of the described embodiments, when the CTLD is tetranectin, the amino acid modifications can further compr random substitution of Lysine-148 to Alanine or in Loop 4.

With respect to scheme (e), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise a modification that combines two Loops into a single Loop, wherein the two combined Loops are Loop 3 and Loop 4. In certain embodiments, when the members of the combinatorial library have the modified CTLD of Scheme (e), the amino acid modifications comprise random substitution of at least six amino acids within Loop 3 and random substitution of at least four amino acids within Loop 4. In one specific embodiment, the amino acid modifications comprise random substitution of six amino acids within Loop 3 and random substitution of four amino acids within Loop 4. In any of the embodiments for scheme (e), when the CTLD is from human tetranectin, the amino acid modifications can further comprise random substitution of Proline-144. In one specific embodiment, when the CTLD is from human tetranectin, the amino acid modifications comprise random substitution of six amino acids within Loop 3, random substitution of four amino acids within Loop 4, and a random substitution of proline 144, resulting in a combined Loop 3 and Loop 4 amino acid sequence, comprising, for example, NWEXXXXXXX XGGXXXN (SEQ ID NO: 578), wherein X is any amino acid and wherein the amino acid sequence of SEQ ID NO: 578 forms a single Loop region. Thus, in one specific embodiment, the polypeptide members of the combinatorial library comprise the sequence NWEXXXXXXX XGGXXXN (SEQ ID NO: 578), wherein X is any amino acid and wherein the amino acid sequence of SEQ ID NO: 578 forms a single loop from combined and modified Loop 3 and Loop 4.

With respect to scheme (f), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise at least one amino acid insertion in Loop 4 and random substitution of at least three amino acids within Loop 4. In certain embodiments, the amino acid modifications comprise four amino acid insertions in Loop 4. In one embodiment, the amino acid modifications comprise at least four amino acid insertions in Loop 4 and random substitution of at least three amino acids within Loop 4. In one specific embodiment, the amino acid substitutions comprise four amino acid insertions in Loop 4 and random substitution of three amino acids within Loop 4.

With respect to scheme (g), the polypeptide members of the combinatorial library comprise a modified Loop 3 and a modified Loop 5, wherein the modified Loop 3 comprises randomization of five amino acid residues and the modified Loop 5 comprises randomization of three amino acid residues. In one embodiment, the polypeptide members of the combinatorial library comprise a modified Loop 3, a modified Loop 5, and a modified Loop 4, wherein the modification to Loop 4 abrogates plasminogen binding. For example, when the combinatorial library has the modified CTLD of Scheme (g), and the CTLD is from tetranectin, the amino acid modifications can further comprise one or more amino acid modifications in Loop 4 that modulates plasminogen binding affinity of the CTLD, for example, the substitution of Lysine 148 to Alanine. Thus, in certain embodiments, when the CTLD is from human or mouse tetranectin, the amino acid modifications comprise random substitution of at least five amino acid residues in Loop 3, random substitution of at least three amino acid residues in Loop 5, and substitution of Lysine 148 to Alanine in Loop 4. In one specific embodiment, the amino acid modifications comprises random substitution of five amino acid residues in Loop 3 and random substitution of three amino acid residues in Loop 5, and, in another specific embodiment, when the CTLD is from human or mouse tetranectin, the amino acid modifications further comprise substitution of Lysine 148 to Alanine in Loop 4.

With respect to scheme (h), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise random substitution of at least one amino acid and at least six amino acid insertions. In certain embodiments, when the CTLD is from tetranectin, the amino acid modifications can further comprise one or more amino acid modifications in Loop 4 that modulates plasminogen binding affinity of the CTLD, for example, the substitution of lysine 148 to Alanine. In certain embodiments when the CTLD is from human or mouse tetranectin, the members of the combinatorial library have random substitution of at least one amino acid and insertion of at least six amino acids in Loop 3, and substitution of Lysine 148 to Alanine in Loop 4. In one specific embodiment, the amino acid modifications comprise random substitution of one amino acid and insertion of six amino acids in Loop 3. In one specific embodiment, when the CTLD is from human or mouse tetranectin, the members of the combinatorial library have random substitution of one amino acid and insertion of six amino acids in Loop 3, and substitution of lysine 148 to alanine in Loop 4. In any of these embodiments when the CTLD is from human or mouse tetranectin, one of the substitutions is the substitution of Isoleucine 140.

With respect to scheme (i), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications comprise a mixture of random substitution of six amino acids in Loop 3 and random substitution of six amino acids and one amino acid insertion in Loop 3. In one embodiment, the mixture further comprises random substitution of six amino acids and two amino acid insertions in Loop 3. Thus in one embodiment, the amino acid modifications comprises a mixture of random substitution of six amino acids in Loop 3, random substitution of six amino acids and one amino acid insertion in Loop 3, and random substitution of six amino acids and two amino acid insertions in Loop 3. In any of the embodiments of scheme (i), when the CTLD is from tetranectin, the amino acid modifications further comprise a substitution of Lysine 148 to Alanine in Loop 4.

With respect to scheme (i), the invention provides a combinatorial polypeptide library comprising polypeptide members that have a randomized C-type lectin domain (CTLD), wherein the randomized CTLD comprises amino acid modifications in at least one of the four loops in the loop segment A (LSA) of the CTLD, wherein the amino acid modifications in at least one of the four loops in the loop segment A (LSA) of the CTLD, wherein the amino acid modifications comprise at least four or more amino acid insertions in at least one of the four loops in the loop segment A (LSA) or loop 5 in loop segment B (LSB) of the CTLD.

In embodiments wherein the combinatorial library comprises one or more amino acid modifications to the Loop 4 region (alone or in combination with modifications to other regions of the CTLD), certain of the modification(s) are designed to maintain, modulate, or abrogate the metal ion-binding affinity of the CTLD. Such modifications affect the plasminogen-binding activity of the CTLD (see, e.g., Nielbo, et al., Biochemistry, 2004, 43 (27), pp 8636-8643; or Graversen 1998).

The polypeptide members of the libraries can comprise one or more amino acid modifications (e.g., by insertion, substitution, extension, or randomization) in any combination of the four LSA loops and the LSB loop (Loop 5) of the CTLD. Thus, in any of the various embodiments described herein, the randomized CTLD can comprise one or more amino acid modifications in the loop of the LSB loop region (Loop 5), either alone, or in combination with one or more amino acid modifications in any one, two, three, or four loops of the LSA loop region (Loops 1-4). In one aspect, the invention provides a combinatorial polypeptide library comprising polypeptide members that have a randomized C-type lectin domain (CTLD), wherein the randomized CTLD comprises one or more amino acid modifications in at least one of the four loops in loop segment A (LSA) and one or more amino acid modifications in the loop in loop segment B (LSB)(Loop 5) of the CTLD, wherein the one or more amino acid modifications comprises randomization of the LSB amino acid residues.

According to the various embodiments described herein, the polypeptide members of the combinatorial libraries can have one or more amino acid modifications in any two, three, four, or five loops in the loop region (LSA and LSB) of the CTLD (e.g., any random combination of random amino acid modifications to two loops, to three loops, to four loops, or to all five loops). The polypeptide members of the combinatorial libraries can further comprise additional amino acid modifications to regions of the CTLD outside of the loop region (LSA and LSB), such as in the α-helices or β-strands (see, e.g., FIG. 1).

In further embodiments of the invention, the CTLD loop regions can be extended beyond the exemplary constructs detailed in the non-limiting Examples below.

In one aspect, the invention also provides a library of nucleic acid molecules encoding polypeptides of the combinatorial polypeptide library according to any one of the above-described aspects and embodiments. In one embodiment of this aspect, the invention provides a library of nucleic acid sequences encoding the polypeptides of the library, wherein the CTLDs of the polypeptides have been modified according to Schemes (a)-(j).

Generating Recombinant CTLD Modified Loop Libraries

In one aspect, the invention provides methods for generating a polypeptide library comprising polypeptide members that have a C-type lectin domain (CTLD), wherein the CTLD comprises one or more amino acid modifications in at least one of the four loops in loop segment A (LSA) and/or in the loop in loop segment B (LSB)(Loop 5) of the CTLD.

In embodiments of this aspect, the method comprises generating at least one random mutation in at least one of the four loops in the LSA region and/or in the loop in the LSB region of the CTLD, wherein the at least one random mutation comprises (a) an insertion of one or more amino acids in the at least one loop; or (b) a substitution of one or more amino acids within or immediately adjacent to the at least one loop; or (c) a deletion of one or more amino acids within or immediately adjacent to the at least one loop; (d) a modification that combines two adjacent loops, or (e) any combination thereof.

In certain embodiments of this aspect, the method comprises generating random mutations in at least one of the four loops in the LSA region and/or in the loop in the LSB region of the CTLD in accordance with any of Schemes (a)-(j).

In certain embodiments of this aspect, the polypeptides of the recombinant CTLD libraries comprise modified CTLDs in which certain Ca²⁺ coordinating amino acid(s) in the loop regions is retained and/or comprise modified CTLDs in which plasminogen binding activity is eliminated.

Also, in certain embodiments of this aspect, the recombinant CTLD libraries can comprise polypeptides having modified CTLD regions, wherein the amino acid modifications fall outside of the loop region (LSA and LSB) of the CTLD. Accordingly, such modifications can be designed or randomly generated in any one or more of the beta strand and/or alpha helical regions.

Generating randomized and optimized recombinant CTLD libraries to obtain protein products that can bind specifically to targets of interest can be performed by any technique known in the art such as, for example, oligonucleotide-directed randomization, error-prone PCR mutagenesis, DNA shuffling by random fragmentation, loop shuffling, loop walking, somatic hypermutation (see, e.g., US Patent Publication 2009/0075378, which is incorporated by reference), and other known methods in the art to create sequence diversity in order to generate molecules with optimal binding activity. (See, e.g., Stemmer, W. P., Proc Natl Acad Sci USA, (October 1994) 91:10747-751; Patrick, W. M. & Firth, A. E., Biomolecular Engineering, (2005) 22:105-112; Firth, A. E. & Patrick, W. M., Bioinformatics, (2005) 21(15):3314-3315; and Lutz S. & Patrick, W. M., Curr. Opin. Biotechnol., (2004) 15:291-297).

In certain embodiments, the generating and optimizing methods comprise an oligonucleotide-directed randomization (NNK or NNS) strategy for mutagenizing the loops. For example, the human tetranectin (hTN) CTLD shown in FIG. 1 and FIG. 4 contains five loops (four loops in LSA and one loop in LSB), which can be altered to confer binding of the CTLD to any target molecule(s) of interest. Random amino acid sequences (generated via randomization, substitution, insertion, etc) can be introduced into one or more of these loops to create libraries from which CTLD domains with the desired binding properties can be selected. Construction of these libraries containing random peptides constrained within any or all of the five loops of the human tetranectin CTLD can be accomplished using either a NNK or NNS as described herein. These libraries can comprise further amino acid modifications that are introduced in regions of the CTLD that are outside of the LSA or LSB regions (e.g., the α-helices and/or β-strands). The following procedure describes a non-limiting, illustrative example of a method by which seven random peptides can be inserted into loop 1 of the hTN CTLD.

PCR can be used to generate a first fragment (fragment A, see FIG. 7) using the following strategy. Forward oligo 1Xfor (5′-GG CTG GGC CTG AAC GAC ATG NNK NNK NNK NNK NNK NNK NNK TGG GTG GAT ATG ACT GGC GCC-3′; SEQ ID NO: 137) wherein N=A, T, G or C, and K=G or T, encodes the region surrounding loop 1 of the CTLD, but replaces 15 nucleotides coding for five amino acids (AAEGT; SEQ ID NO: 579) of loop 1 with seven NNK codons. These NNK codons encoding seven random amino acids replace the wild type codons encoding the five native tetranectin amino acids. Oligo 1Xfor (SEQ ID NO: 137) can be annealed with the reverse oligo 1Xrev2 (5′-GGC GGT GAT CTC AGT TTC CCA GTT CTT GTA GGC GAT GCG GGC GCC AGT CAT ATC CAC CCA-3′; SEQ ID NO: 580). The two oligos are complementary across 21 nucleotides of their 3′ ends. Referring to FIG. 7, PCR is used to generate Fragment A (101 bp) from these two overlapping oligos. Similarly, a Fragment B (see FIG. 7) can be created by performing PCR using forward oligo BstX1 for (5′-ACT GGG AAA CTG AGA TCA CCG CCC AAC CTG ATG GCG GCG CAA CCG AGA ACT GCG CGG TCC TG-3′; SEQ ID NO: 139) and the reverse primer PstBssRevC (5′-CCC TGC AGC GCT TGT CGA ACC ACT TGC CGT TGG CGG CGC CAG ACA GGA CCG CGC AGT TCT-3′; SEQ ID NO: 140) to generate a 105 bp fragment. PCR can be performed using a high fidelity polymerase or taq blend and standard PCR thermocycling conditions. The 3′ end of fragment A is complementary to the 5′ end of fragment B. These fragments can be gel isolated and subsequently combined for overlap extension PCR using outer primers Bglfor12 (SEQ ID NO: 141) and PstRev (SEQ ID NO: 142). The resulting 195 bp fragment can be gel isolated and then digested with the restriction enzymes Bgl II and Pst I, after which the final 185 bp fragment can be gel isolated and cloned into a phage display vector (such as CANTAB 5E) containing the restriction modified CTLD shown below fused to Gene III, which is similarly digested with Bgl II and Pst I for cloning.

Modification of other loops by replacement with randomized amino acids can be similarly performed as described herein. The replacement of defined amino acids within a loop with randomized amino acids is not restricted to any specific loop, nor is it restricted to the original size of the loops. Likewise, total replacement of the loop is not required, partial replacement is possible for any of the loops. In some cases retention of some of the original amino acids within the loop, such as the calcium coordinating amino acids, may be desirable. In these cases, replacement with randomized amino acids may occur for either fewer of the amino acids within the loop to retain the calcium coordinating amino acids, or additional randomized amino acids may be added to the loop to increase the overall size of the loop yet still retain these calcium coordinating amino acids. Very large peptides can be accommodated and tested by combining loop regions, such as loops 1 and 2 or loops 3 and 4, into one larger replacement loop.

The nucleic acid molecules can be obtained by ordinary methods for chemical synthesis of nucleic acids by directing the step-wise synthesis to add pre-defined combinations of pure nucleotide monomers or a mixture of any combination of nucleotide monomers at each step in the chemical synthesis of the nucleic acid fragment. In this way it is possible to generate any level of sequence degeneracy, from one unique nucleic acid sequence to the most complex mixture, which will represent a complete or incomplete representation of maximum number unique sequences of 4^{N}, where N is the number of nucleotides in the sequence.

Complex compositions comprising a plurality of nucleic acid fragments can, alternatively, be prepared by generating mixtures of nucleic acid fragments by chemical, physical or enzymatic fragmentation of high-molecular mass nucleic acid compositions such as, for example, genomic nucleic acids extracted from any organism. To render such mixtures of nucleic acid fragments useful in the generation of recombinant libraries, as described here, the crude mixtures of fragments, obtained in the initial cleavage step, would typically be size-fractionated to obtain fragments of an approximate molecular mass range which would then typically be adjoined to a suitable pair of linker nucleic acids, designed to facilitate insertion of the linker-embedded mixtures of size-restricted oligonucleotide fragments into the receiving nucleic acid vector.

Nucleic acid fragments can be inserted in specific locations into receiving nucleic acids by any common method of molecular cloning of nucleic acids, such as by appropriately designed PCR manipulations in which chemically synthesized nucleic acids are copy-edited into the receiving nucleic acid, in which case no endonuclease restriction sites are required for insertion. Alternatively, the insertion/excision of nucleic acid fragments may be facilitated by engineering appropriate combinations of endonuclease restriction sites into the target nucleic acid into which suitably designed oligonucleotide fragments may be inserted using standard methods of molecular cloning of nucleic acids.

After rounds of selection on specific targets (e.g. eukaryotic cells, virus, bacteria, specific proteins, polysaccharides, other polymers, organic compounds etc.) DNA is isolated from the specific phages, and the nucleotide sequence of the segments encoding the ligand-binding region determined, excised from the phagemid DNA and transferred to the appropriate derivative expression vector for heterologous production of the desired product. Heterologous production in a prokaryote can be used for the isolation of the desired product.

To facilitate the construction of combinatorial CTLD libraries, restriction sites can be introduced into the CTLD. For example, suitable restriction sites located in the vicinity of the nucleic acid sequences encoding β2, β3 and β4 in both human and murine tetranectin were designed with minimal perturbation of the polypeptide sequence encoded by the altered sequences. It was found possible to establish a design strategy, as detailed below, by which identical endonuclease restriction sites could be introduced at corresponding locations in the two sequences, allowing interesting loop-region variants to be readily excised from a recombinant murine CTLD and inserted correctly into the CTLD framework of human tetranectin or vice versa.

Analysis of the nucleotide sequence encoding the mature form of human tetranectin (FIG. 2) reveals that a recognition site for the restriction endonuclease Bgl II is found at position 326 to 331 (AGATCT), involving the encoded residues Glu109, Ile110, and Trp111 of β2, and that a recognition site for the restriction endonuclease Kas I is found at position 382 to 387 (GGCGCC), involving the encoded amino acid residues Gly128 and Ala129 (located C-terminally in loop 2). By utilizing alternate codons for naturally occurring amino acids in the tetranectin sequence, the restriction endonuclease sites Pst I (CTGCAG) and Mfe I (CAATTG) were engineered into the tetranectin coding sequence at positions 501 to 506 (CTGCCG, originally), involving the encoded amino acid residues Arg167, Cys168, and Arg169, and positions 511 to 516 (CAGCTG, originally), involving the encoded amino acid residues Gln171 and Leu172, all located between β4 and β5.

In certain other aspects of the invention, nucleic acid constructs in the form of plasmids, vectors, transcription or expression cassettes which comprise at least one nucleic acid described herein are provided. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press.

The invention also provides a recombinant host cell which comprises one or more of the constructs as described herein. Suitable host cells include bacteria, mammalian cells, yeast, and baculovirus systems. Mammalian cell lines available in the art for expression of a heterologous polypeptide include Chinese hamster ovary cells, HeLa cells, baby hamster kidney cells, NSO mouse melanoma cells and many others. In one embodiment the host cell is HEK293 cells.

Display Systems

The resulting recombinant CTLD libraries described herein can be displayed using a number of alternative techniques that are described herein and known in the art. Methods for expressing the nucleic acid molecule library in a display system are described in US Patent Application Publication 2007/0275393, which is incorporated by reference herein in its entirety. In one embodiment, the display system comprises an observable phenotype that represents at least one property of the displayed expression products and the corresponding genotypes. Examples of suitable display systems include a phage display system; a yeast display system; a viral display system; a cell-based display system; a ribosome-linked display system; or a plasmid-linked display system; any combinations thereof, or any other suitable display system that is known in the art.

Thus, in one aspect, the invention provides a display system comprising the combinatorial polypeptide library according to any one of the above-described aspects and embodiments. In one embodiment of this aspect, the invention provides a display system comprising the combinatorial polypeptide library according to Schemes (a)-(i).

In certain embodiments of this aspect, the display system comprises a phage display system; a yeast display system; a viral display system; a cell-based display system; a ribosome-linked display system; or a plasmid-linked display system; any combinations thereof, or any other display system that is known in the art.

Several systems displaying phenotype, in terms of putative ligand binding modules or modules with putative enzymatic activity, have been described. These include: phage display (e.g., the filamentous phage fd (Dunn (1996); Griffiths and Duncan (1998); Marks et al. (1992)), phage lambda display (Mikawa et al. (1996)), display on eukaryotic virus (e.g., baculovirus (Ernst et al. (2000))), cell display (e.g., display on bacterial cells (Benhar et al. (2000))), yeast cells (Boder and Wittrup (1997)), and mammalian cells (Whitehorn et al. (1995)), ribosome linked display (Schaffitzel et al. (1999)), and plasmid linked display (Gates et al. (1996)).

A commonly used method for phenotype display and linking this to genotype is by phage display. This is accomplished by insertion of the reading frame encoding the scaffold protein or protein of interest to a surface exposed phage protein. The filamentous phage fd (e.g. M13) has proven useful for this purpose.

US Patent Application Publication No: 2007/0275393 describes a procedure for accomplishing a display system for the generation of CTLD libraries. In general, a method for generating a display system for the described CTLD libraries comprises:

(1) identifying the location of the loop-region of a CTLD;

(2) subcloning a nucleic acid fragment encoding the CTLD of choice into a protein display vector system with or without prior insertion of endonuclease restriction sites close to the sequences encoding β2, β3 and β4 in the CTLD; and

(3) substituting the nucleic acid fragment encoding some or all of the loop-region of the CTLD of choice with randomly selected members of an ensemble consisting of a multitude of nucleic acid fragments, resulting in randomization and/or extension of the original loop region of the CTLD. Each of the cloned nucleic acid fragments, encoding a new polypeptide with a substituted loop segment or entire loop region, will be decoded in the reading frame determined within its new sequence context.

The location of the loop region of a CTLD can be identified using the methods previously described herein. Briefly, the loop region can be identified by referring to the three dimensional structure of the CTLD of choice, if such information is available, or, if not, identifying the sequence locations of the β2-, β3- and β4-strands by sequence alignment with the sequences shown in FIG. 1, as aided by the identification of sequence elements corresponding to the β2 and β3 consensus sequence elements and β4-strand characteristics, and the conserved cysteine residues also disclosed herein in FIG. 1.

Strategies for Identifying and Isolating CTLD polypeptides that bind to target molecules

In one aspect, the invention provides a method for identifying and isolating a polypeptide having specific binding activity to a target molecule, wherein the method comprises (a) providing a combinatorial polypeptide library of the invention; (b) contacting the polypeptides of the combinatorial polypeptide library with the target molecule under conditions that allow for binding between a polypeptide and the target molecule; and (c) isolating a polypeptide that binds to the target molecule. In various embodiments, the target molecule can comprise any molecule associated with the surface of a cell (such as eukaryotic cells, tumor cells, immune cells, bacterial cells, protozoa, fungi and a cell infected with a virus); proteins (such as receptor proteins, soluble proteins, enzymes, or antibodies); polysaccharides; polymers; and small organic compounds.

In another aspect, the invention provides a method for identifying and isolating a polypeptide having specific binding activity to a target molecule, wherein the method further comprises a library of nucleic acid molecules encoding polypeptides of the combinatorial polypeptide library, wherein the library of nucleic acids is expressed in a display system. In one embodiment, the display system comprises an observable phenotype that represents at least one property of the displayed expression products and the corresponding genotypes.

In another aspect, the invention provides a method for identifying and isolating a polypeptide having specific binding activity to a target molecule comprising the steps of: (a) providing a library of nucleic acid molecules encoding the polypeptide library of claim 1; (b) expressing the library of nucleic acid molecules in a display system to obtain an ensemble of polypeptides, in which the amino acid residues at one or more sequence positions differ between different members of said ensemble of polypeptides; (c) contacting the ensemble of polypeptides with said target molecule under conditions that allow for binding between a polypeptide and the target molecule; and (d) isolating a polypeptide that is capable of binding to said target molecule.

In any of these aspects and embodiments, the invention provides a method for identifying and isolating a polypeptide having specific binding activity to a target molecule, wherein the polypeptide has been modified in accordance with any of Schemes (a)-(i).

A specific binding member for a target molecule of interest can be obtained from a random library of polypeptides by selection of members of the library that specifically bind to the target molecule. As discussed herein, a number of systems for displaying phenotypes with putative ligand binding sites are known. These include: phage display (e.g. the filamentous phage fd [Dunn (1996), Griffiths and Duncan (1998), Marks et al. (1992)], phage lambda [Mikawa et al. (1996)]), display on eukaryotic virus (e.g. baculovirus [Ernst et al. (2000)]), cell display (e.g. display on bacterial cells [Benhar et al. (2000)], yeast cells [Boder and Wittrup (1997)], and mammalian cells [Whitehorn et al. (1995)], ribosome linked display [Schaffitzel et al. (1999)], and plasmid linked display [Gates et al. (1996)].

To select for polypeptides with binding activity to a target molecule, libraries can be constructed and initially screened for binding to the target molecule as monomeric elements, either as single monomeric CTLD domains or individual peptides displayed on the surface of phage. Libraries can be constructed by randomizing the amino acids in one or more of the five different loops (or outside the loops) within the CTLD scaffold displayed on the surface of phage. Binding to the target molecules can be selected for by phage display panning.

Several strategies can be employed in the construction of phage display libraries. One strategy is to construct and/or use random peptide phage display libraries. Random linear peptides and/or random peptides constructed as disulfide constrained loops can be individually displayed on the surface of phage particles and selected for binding to the desired target molecule through phage display “panning”. After obtaining peptide clones with the desired binding activity, these peptides can be grafted on to the trimerization domain of human tetranectin or into loops of the CTLD domain followed by grafting on the trimerization domain and screened for agonist activity.

Another strategy for construction of phage display libraries and trimerization domain constructs include obtaining CTLD derived binders. Libraries can be constructed by randomizing the amino acids in one or more of the five different loops within the CTLD scaffold (i.e., of human tetranectin) displayed on the surface of phage. Binding to the target molecule can be selected for through phage display panning. After obtaining CTLD clones with peptide loops demonstrating the desired binding activity, the CTLD clones can then be grafted on to the trimerization domain of human tetranectin and screened for agonist activity.

Another strategy includes using peptide sequences with known binding capabilities to the target of interest and first improving their binding by creating new libraries with randomized amino acids flanking the peptide or/and randomized selected internal amino acids within the peptide, followed by selection for improved binding through phage display. After obtaining binders with improved affinity, the binders of these peptides can be fused to other functional protein domains such as, for example, the trimerization domain of human tetranectin (discussed herein below and discussed in detail in PCT/US09/60271 and US. 2010/0028995, which are incorporated herein by reference in their entirety), and evaluated for desired activity. In this method, initial libraries can be constructed as either free peptides displayed on the surface of phage particles, as in the first strategy, or as constrained loops within the CTLD scaffold as in the second strategy discussed above. These display strategies are described in detail in PCT/US09/60271, which is incorporated by reference herein in its entirety.

Exemplary strategies for identifying and isolating polypeptides having specific binding activity with a target molecule of interest are described in further detail below. Although these strategies focus on phage display, other equivalent methods of identifying polypeptides can be used.

Strategy 1

Peptide display library kits such as, but not limited to, the New England Biolabs Ph.D. Phage display Peptide Library Kits are sold commercially and can be purchased for use in selection of new and novel peptides with specific binding activity for a target molecule of interest. Three forms of the New England Biolabs kit are available: the Ph.D.-7 Peptide Library Kit containing linear random peptides 7 amino acids in length, with a library size of 2.8×10⁹ independent clones, the Ph.D.-C7C Disulfide Constrained Peptide Library Kit containing peptides constructed as disulfide constrained loops with random peptides 7 amino acids in length and a library size of 1.2×10⁹ independent clones, and the Ph.D.-12 Peptide Library Kit containing linear random peptides 12 amino acids in length, with a library size of 2.8×10⁹ independent clones.

Alternatively similar libraries can be constructed de novo with peptides containing random amino acids similar to these kits. For de novo construction, random nucleotides can be generated using either an NNK, or NNS strategy, in which N represents an equal mixture of the four nucleic acid bases A, C, G and T. The K represents an equal mixture of either G or T, and S represents and equal mixture of either G or C. These randomized positions can be cloned onto the Gene III protein in either a phage or phagemid display vector system. Both the NNK and the NNS strategy cover all 20 possible amino acids and one stop codon with slightly different frequencies for the encoded amino acids. Because of the limitations of bacterial transformation efficiency, library sizes generated for phage display are in the order of those started above, thus peptides containing up to 7 randomized amino acids positions can be generated and yet cover the entire repertoire of theoretical combinations (20⁷=1.28×10⁹). Longer peptide libraries can be constructed using either the NNK or NNS strategy however the actual phage display library size likely will not cover all the theoretical amino acid combinations possible associated with such lengths due to the requirement for bacterial transformation.

Thus ribosome display libraries might be beneficial where larger/longer random peptides are involved. For disulfide constrained libraries, a similar NNK or NNS random nucleotide strategy can be used. However, these random positions are flanked by cysteine amino acid residues, to allow for disulfide bridge formation. The N-terminal cysteine is often preceded by an additional amino acid such as alanine. In addition a flexible linker made up of but not limited to several glycine residues may act as a spacer between the peptides and the gene III protein for any of the above random peptide libraries.

Strategy 2

The human tetranectin CTLD shown in FIGS. 1 and 4 contains five loops (four loops in LSA and one loop comprising LSB), which can be altered to confer binding of the CTLD to different protein targets. Random amino acid sequences can be placed in one or more of these loops to create libraries from which CTLD domains with the desired binding properties can be selected. For example, any of the CTLD polypeptide libraries described herein can be used, i.e., polypeptides having CTLDs modified in accordance with any of Schemes (a)-(i). Construction these libraries containing random peptides constrained within any or all of the five loops of the human tetranectin CTLD can be accomplished (but is not limited to) using either a NNK or NNS as described above in strategy 1 and also described in detail elsewhere herein.

Strategy 3

In instances where other peptides with binding activity to the target molecule of interest have been identified, a strategy can be utilized in which these peptides can be cloned directly on to either the N- or C-terminal end of the trimerization domain of tetranectin as free linear peptides or as disulfide constrained loops using cysteines can be utilized. Single-chain antibodies or domain antibodies capable of binding to the target of interest can also be cloned on to either end of the trimerization domain. Additionally, peptides with known binding properties can be cloned directly into any one of the loop regions of the TN CTLD. Peptides selected as disulfide constrained loops or as complementarity-determining regions of antibodies might be quite amenable to relocation into the loop regions of the CTLD of human tetranectin. Binding can be tested for all of these constructs in monomeric form, and binding and agonist activation can be tested in trimeric form, when the CTLD is fused with the trimerization domain

CTLD Polypeptides

The combinatorial polypeptide libraries of the invention can be used to generate and identify polypeptides comprising CTLDs with desired binding properties to target molecules of interest.

In one aspect, the invention provides a polypeptide having the scaffold structure of a C-type Lectin Like Domain (CTLD), wherein the polypeptide binds to a target other than a natural target for that CTLD and wherein the CTLD scaffold structure of the CTLD is modified according to any of the schemes (a)-(j). In one embodiment, the CTLD scaffold structure is modified according to any of the schemes (a)-(j) and further comprises any of the further modifications described herein, for example, modifications outside the CTLD loop region. In one embodiment, the polypeptide has the scaffold structure of the CTLD from human or mouse tetranectin and binds to a target other than plasminogen.

The CTLD polypeptide of the invention can be produced using any of the methods and combinatorial libraries described herein. For example, in one embodiment, the polypeptide can be produced using a combinatorial library of polypeptides having a CTLD, wherein the loop region of the CTLD is randomized according to any of the Schemes (a)-(j), contacting the combinatorial polypeptide library with the target molecule under conditions that allow for binding between a polypeptide and the target molecule; and isolating a polypeptide that binds to the target molecule, wherein the target molecule is not the natural target for that CTLD. In one embodiment of this method, the CTLD is human or mouse tetranectin. In another embodiment of this method, the CTLD is randomized according to any of the Schemes (a)-(j) and comprises any of the further modifications described herein, for example, modifications outside the CTLD loop region.

A non-natural target for a modified CTLD according to the invention can be any chemical compound in free or conjugated form which exhibits features of an immunological hapten, a hormone such as steroid hormones, or any biopolymer or fragment thereof, for example, a protein or protein domain; a peptide; an oligodeoxynucleotide; a nucleic acid; arachidonic acid or its metabolites, lipids or metabolites thereof; fatty acids or metabolites thereof; free radicals; an oligo- or polysaccharide or conjugates thereof; or chemically synthesized or natural drugs of abuse or therapeutic use. In one aspect, the target is a protein. The protein can be any globular soluble protein or a receptor protein, for example, a trans-membrane protein involved in cell signaling, a component of the immune systems such as an MHC molecule or cell surface receptor that is indicative of a specific disease. The protein can be a post translationally modified protein having the addition of a biochemical functional group such as acetate, phosphate, and/or various lipids and carbohydrates, including but not limited to, glycosylation and myristoylation. The modified CTLD of the invention can also bind protein fragments. For example, the CTLD can bind to a domain of a cell surface receptor, when it is part of the receptor anchored in the cell membrane as well as to the same domain in solution, if this domain can be produced as a soluble protein as well. The CTLDs can also have specific binding affinity to ligands of low(er) molecular weight such as biotin, fluorescein or digoxigenin.

In various embodiments, the CTLD polypeptide sequences that bind one or more target molecule(s) can have binding affinities that are about equal to the binding affinities of naturally occurring ligands for the one or more target molecule(s). In certain embodiments, the polypeptides of the invention have a binding affinity for one or more target molecule(s) that is stronger than the binding affinity that a native ligand has for the same target molecule(s). Such polypeptides are useful, for example, for blocking the activity of binding members in some cases, or for more potently agonizing in other cases, e.g., in cases in which the modified CTLD binds to a receptor and is further selected to agonize the receptor. In other embodiments, the polypeptides of the invention have a binding affinity for one or more target molecule(s) that is weaker than the binding affinity that a native ligand has for the same target molecule(s). CTLD polypeptides having a weaker affinity for a target molecule(s) than a native ligand may have an improved ability to penetrate tumors or tissues and/or may be useful in cases where the desired goal is to dampen the activity of the target rather than completely block it. CTLDs with a lower binding affinity over a native ligand could also be desired, for example, in cases where the optimal selected activity is based on internalization into the cell following binding to the target.

The modified CTLDs can also bind to one or more receptor(s) and act as agonists. In such embodiments, the respective binding affinity of the agonists can be determined and compared to the binding properties of native ligands, or a portion thereof, by ELISA, RIA, and/or BIAcore assays, as well as other assays known in the art. In certain embodiments, the receptor-selective agonists of the invention inhibit or induce a biological activity in at least one type of mammalian cell (e.g., a cancer cell), and such activity can be determined by known art methods. Examples of CTLDs identified using the methods provided herein that act as agonists are polypeptides that bind to TRAIL-R1 and TRAIL-R2.

In other embodiments, the modified CTLDs can bind to one or more receptor(s) or one or more ligand(s) having affinity for a receptor(s) and act as antagonists (receptor blockers). In such embodiments, the respective binding affinity of the agonists can be determined and compared to the binding properties of native ligands, or a portion thereof, by ELISA, RIA, and/or BIAcore assays, as well as other assays known in the art. In certain embodiments, the antagonists of the invention inhibit or induce a biological activity in at least one type of mammalian cell (e.g., a cancer cell), and such activity can be determined by known art methods. Examples of CTLDs identified using the methods provided herein that act as antagonists are polypeptides that bind to IL-23R.

Polypeptides comprising CTLDs that specifically bind to a target molecule of interest can comprise a “binding member”, which includes all or a portion of the CTLD. The term “binding member” as used herein refers to a member of a pair of molecules which have binding specificity for one another. The members of a binding pair may be naturally derived or wholly or partially synthetically produced. One member of the pair of molecules has an area on its surface, or a cavity, which binds to and is therefore complementary to a particular spatial and polar organization of the other member of the pair of molecules. Thus the members of the pair have the property of binding specifically to each other.

In embodiments wherein the CTLD-based protein products are derived from a mammalian tetranectin, as exemplified herein with murine and human tetranectin, the structure is nearly identical with all other mammalian tetranectins. This species-conserved structure allows for straightforward swapping of polypeptide segments defining ligand-binding specificity between orthologs (e.g. murine and human tetranectin derivatives). Thus, in such embodiments, this platform provides a particular advantage over the “humanization” of murine antibody derivatives, which can involve a number of complications.

In one aspect, the invention provides a polypeptide having a multimerizing domain and comprises at least one CTLD polypeptide-binding member that binds to at least one target molecule. As used herein, the term “multimerizing domain” means an amino acid sequence that comprises the functionality that can associate with two or more other amino acid sequences to form trimers or other multimeric complexes. In various embodiment so of the invention, the multimerizing domain is a dimerizing domain, a trimerizing domain, a tetramerizing domain, a pentamerizing domain, etc. These domains are capable of forming polypeptide complexes of two, three, four, five or more polypeptides of the invention.

In one example, the polypeptide contains an amino acid sequence—a “trimerizing domain”—which forms a trimeric complex with two other trimerizing domains. A trimerizing domain can associate with other trimerizing domains of identical amino acid sequence (forming a homotrimer), or with trimerizing domains of different amino acid sequence (forming a heterotrimer). The interaction is of the type that produces trimeric proteins or polypeptides. Such an interaction may be caused by covalent bonds between the components of the trimerizing domains as well as by hydrogen bond forces, hydrophobic forces, van der Waals forces and salt bridges. The trimerizing effect of trimerizing domain is caused by a coiled coil structure that interacts with the coiled coil structure of two other trimerizing domains to form a triple alpha helical coiled coil trimer that is stable even at relatively high temperatures. In various embodiments, for example, a trimerizing domain based upon a tetranectin structural element, the complex is stable at least 60° C., for example in some embodiments at least 70° C.

In one embodiment, the multimerized polypeptide is a trimer, for example a tetranectin trimerizing module (see US 2007/0154901). A trimeric complex including a CTLD is referred to herein as an “atrimer.” An “ATRIMER™” polypeptide complex refers to a trimeric complex of three trimerizing domains that also include CLTDs (Anaphore, Inc., San Diego, Calif.).

In accordance with the invention, a binding member may either be linked to the N- or the C-terminal amino acid residue of the multimerizing domain. Also, in certain embodiments it may be advantageous to have a binding member at both the N-terminus and the C-terminus of the multimerizing domain of the monomer, thereby providing a multimeric polypeptide complex. For example, when the multimeric peptide forms trimers with like molecules, six binding members capable of binding a target molecule of interest can be associated with a single trimeric complex.

In another aspect of the invention, a polypeptide that specifically binds to a target molecule of interest is contained in one or more loops in the loop region of a CTLD. In this aspect, the CTLD can be attached to any known trimerizing domain at the C-terminus of the trimerizing domain. Also, a fusion protein of the invention can include a second CTLD domain, fused at the N-terminus of the trimerizing domain. In a variation of this aspect, the fusion protein includes a polypeptide that binds to a first target molecule at one of the termini of the trimerizing domain and a CTLD at the other of the termini. One, two or three such proteins can be part of a trimeric complex containing up to six specific CTLD binding members for one or more target molecules.

In another aspect, the invention provides a multimeric complex of three proteins, each of the proteins comprising a multimerizing domain and at least one CTLD polypeptide that binds to at least one target molecule of interest. In one embodiment, the multimeric complex comprises a fusion protein having a multimerizing domain selected from a tetranectin trimerizing structural element (tetranectin trimerizing module), a mannose binding protein (MBP) trimerizing domain, a collectin neck region, and other similar moieties. The multimeric complex can be comprised of multimerizing domains that are able to associate with each other to form a multimer. Accordingly, in certain embodiments, the multimeric complex is a homomultimeric complex comprised of proteins having the same amino acid sequences. In other embodiments, the multimeric complex is a heteromultimeric complex comprised of proteins having different amino acid sequences such as, for example, different multimerizing domains, and/or different CTLD polypeptides that bind to a different target molecule. In such embodiments, the CTLD polypeptides may all specifically bind to one target molecule. In other embodiments, the CTLD polypeptides specifically bind to different target molecules. Thus, in certain embodiments, the multimeric complex comprises fusion proteins of the invention, wherein each of the fusion proteins comprise at least one CTLD polypeptide that binds to one target molecule, wherein the polypeptides can be the same or different, and/or at least one CTLD polypeptide that binds to a second target molecule, wherein the second target molecule-binding polypeptide can be the same or different.

The trimerizing domain of a polypeptide of the invention can be derived from tetranectin as described in U.S. Patent Application Publication No. 2007/0154901 ('901 Application), which is incorporated by reference in its entirety. The mature human tetranectin single chain polypeptide sequence is provided herein as SEQ ID NO: 11. Examples of a tetranectin trimerizing domain include the amino acids 17 to 49, 17 to 50, 17 to 51 and 17-52 of SEQ ID NO: 40, which represent the amino acids encoded by exon 2 of the human tetranectin gene, and optionally the first one, two or three amino acids encoded by exon 3 of the gene. Other examples include amino acids 1 to 49, 1 to 50, 1 to 51 and 1 to 52, which represents all of exons 1 and 2, and optionally the first one, two or three amino acids encoded by exon 3 of the gene. Alternatively, only a part of the amino acid sequence encoded by exon 1 is included in the trimerizing domain. In particular, the N-terminus of the trimerizing domain may begin at any of residues 1, 2, 3, 4, 5, 6, 7, 8, 9, 10, 11, 12, 13, 14, 15, 16 and 17 of SEQ ID NO: 40. In particular embodiments, the N terminus is I10 or V17 and the C-terminus is Q47, T48, V49, C(S)50, L51 or K52 (numbering according to SEQ ID NO: 40). See PCT US09/60271, which is incorporated by reference herein in its entirety.

The trimerizing domain can be a tetranectin trimerizing structural element (“TTSE”) having an amino acid sequence of SEQ ID NO: 40 which is a consensus sequence of the tetranectin family trimerizing structural element as more fully described in US 2007/00154901, which is incorporated herein by reference in its entirety. The TTSE embraces variants of a naturally occurring member of the tetranectin family of proteins, and in particular variants that have been modified in the amino acid sequence without adversely affecting, to any substantial degree, the ability of the TTSE to form alpha helical coiled coil trimers. In various aspects of the invention, the trimeric polypeptide according to the invention includes a TTSE as a trimerizing domain having at least 66% amino acid sequence identity to the consensus sequence of SEQ ID NO: 49; for example at least 73%, at least 80%, at least 86% or at least 92% sequence identity to the consensus sequence of SEQ ID NO: 40 (counting only the defined (not X) residues). In other words, at least one, at least two, at least three, at least four, or at least five of the defined amino acids in SEQ ID NO: 40 may be substituted.

In one particular embodiment, the cysteine at position 50 (C50) of SEQ ID NO: 40 can be advantageously mutagenized to serine, threonine, methionine or to any other amino acid residue in order to avoid formation of an unwanted inter-chain disulphide bridge, which can lead to unwanted multimerization. Other known variants include at least one amino acid residue selected from amino acid residue nos. 6, 21, 22, 24, 25, 27, 28, 31, 32, 35, 39, 41, and 42 (numbering according to SEQ ID NO: 40), which may be substituted by any non-helix breaking amino acid residue. These residues have been shown not to be directly involved in the intermolecular interactions that stabilize the trimeric complex between three TTSEs of native tetranectin monomers. In one aspect shown in FIG. 2, the TTSE has a repeated heptad having the formula a-b-c-d-e-f-g (N to C), wherein residues a and d (i.e., positions 26, 30, 33, 37, 40, 44, 47, and 51 may be any hydrophobic amino acid (numbering according to SEQ ID NO: 40).

In further embodiments, the TTSE trimerization domain can be modified by the incorporation of polyhistidine sequence and/or a protease cleavage site, e.g, Blood Coagulating Factor Xa or Granzyme B (see US 2005/0199251, which is incorporated herein by reference), and by including a C-terminal KG or KGS sequence. Also, to assist in purification, Proline at position 2 may be substituted with Glycine.

Particular non-limiting examples of TTSE truncations and variants are shown in PCT US09/60271 (FIGS. 3A-3D). In addition, a number of trimerizing domains having substantial homology (greater than 66%) to the trimerizing domain of human tetranectin known:

TABLE 1
Trimerizing Domains

| | | |
|---|---|---|
| Equus caballus TN-like | KMFEELKSQLDSLAQEVALLKEQQALQTVCL | SEQ ID NO: 66 |
| Cat TN | KMFEELKSQVDSLAQEVALLKEQQALQTVCL | SEQ ID NO: 67 |
| Mouse TN | SKMFEELKNRMDVLAQEVALLKEKQALQTVCL | SEQ ID NO: 68 |
| Rat TN | KMFEELKNRLDVLAQEVALLKEKQALQTVCL | SEQ ID NO: 69 |
| Bovine TN | KMLEELKTQLDSLAQEVALLKEQQALQTVCL | SEQ ID NO: 70 |
| Equus caballus CTLD like | DLKTQVEKLWREVNALKEMQALQTVCL | SEQ ID NO: 71 |
| Canis lupus CTLD | DLKTQVEKLWREVNALKEMQALQTVCL | SEQ ID NO: 72 |
| member A | | |
| Bovine CTLD member A | DLKTQVEKLWREVNALKEMQALQTVCL | SEQ ID NO: 73 |
| Macaca mulatta CTLD | DLKTQIEKLWTEVNALKEIQALQTVCL | SEQ ID NO: 74 |
| member A | | |
| Taeniopygia guttata | DDLKTQIDKLWREVNALKEIQALQTVCL | SEQ ID NO: 75 |
| CTLD member A | | |
| Ornithorhynchus | DLKTQVEKLWREVNALKEMQALQTVCL | SEQ ID NO: 76 |
| anatinus CTLD like | | |
| Rat CTLD member A | DLKSQVEKLWREVNALKEMQALQTVCL | SEQ ID NO: 77 |
| Monodelphis domestica | DLKTQVEKLWREVNALKEMQALQTVCL | SEQ ID NO: 78 |
| CTLD member A | | |
| Shark TN | DDLRNEIDKLWREVNSLKEMQALQTVCL | SEQ ID NO: 79 |
| Taeniopygia guttata | KMIEDLKAMIDNISQEVALLKEKQALQTVCL | SEQ ID NO: 80 |
| TN-like | | |
| Gallus gallus TN | KMIEDLKAMIDNISQEVALLKEKQALQTVCL | SEQ ID NO: 81 |
| Danio rerio CTLD | DDMKTQIDKLWQEVNSLKEMQALQTVCL | SEQ ID NO: 82 |
| member A | | |
| Gallus gallus, CTLD | DDLKTQIDKLWREVNALKEMQALQSVCL | SEQ ID NO: 83 |
| member A | | |
| Mouse CTLD member A | DDLKSQVEKLWREVNALKEMQALQTVCL | SEQ ID NO: 84 |
| Gallus gallus CTLD | DDLKTQIDKLWREVNALKEMQALQSVCL | SEQ ID NO: 85 |
| member A | | |
| Tetraodon | DDVRSQIEKLWQEVNSLKEMQALQTVCL | SEQ ID NO: 86 |
| nigroviridis, unkown | | |
| Xenopus laevis | DLKTQIDKLWREINSLKEMQALQTVCL | SEQ ID NO: 87 |
| MGC85438 | | |
| Tetraodon | EELRRQVSDLAQELNILKEQQALHTVCL | SEQ ID NO: 88 |
| nigroviridis, unkown | | |
| Xenopus laevis,unkown | KMYEELKQKVQNIELEVIHLKEQQALQTICL | SEQ ID NO: 89 |
| Xenopus tropicalis TN | KMYEDLKKKVQNIEEDVIHLKEQQALQTICL | SEQ ID NO: 90 |
| Salmo salar TN | EELKKQIDNIVLELNLLKEQQALQSVCL | SEQ ID NO: 91 |
| Danio rerio TN | EELKKQIDQIIQDLNLLKEQQALQTVCL | SEQ ID NO: 92 |
| Tetraodon | EQMQKQINDIVQELNLLKEQQALQAVCL | SEQ ID NO: 93 |
| nigroviridis, unknown | | |
| Tetraodon | EQMQKQINDIVQELNLLKEQQALQAVCL | SEQ ID NO: 94 |
| nigroviridis, unkown | | |

Other human polypeptides that are known to trimerize include those found in Table 2.

TABLE 2
Trimerizing Polypeptides

| | | |
|---|---|---|
| hTRAF3 | NTGLLESQLSRHDQMLSVHDIRLADMDLRF | SEQ ID |
| | QVLETASYNGVLIWKIRDYKRRKQEAVM | NO: 95 |
| hMBP | AASERKALQTEMARIKKWLTF | SEQ ID |
| | | NO: 96 |
| hSPC300 | FDMSCRSRLATLNEKLTALERRIEYIEAR | SEQ ID |
| | VTKGETLT | NO: 97 |
| hNEMO | ADIYKADFQAERQAREKLAEKKELLQEQL | SEQ ID |
| | EQLQREYSKLKASCQESARI | NO: 98 |
| hcubilin | LTGSAQNIEFRTGSLGKIKLNDEDLSECL | SEQ ID |
| | HQIQKNKEDIIELKGSAIGLPIYQLN | NO: 99 |
| | SKLVDLERKFQGLQQT | |
| hThrombos | LRGLRTIVTTLQDSIRKVTEENKELANE | SEQ ID |
| pondins | | NO: 100 |

Another example of a trimerizing domain is disclosed in U.S. Pat. No. 6,190,886 (incorporated by reference herein in its entirety), which describes polypeptides comprising a collectin neck region. Trimers can then be made under appropriate conditions with three polypeptides comprising the collectin neck region amino acid sequence. A number of collectins are identified, including:

Collectin neck region of human SP-D:

| | |
|---|---|
| VASLRQQVEALQGQVQHLQAAFSQYKK | [SEQ ID NO: 101] |

Collectin neck region of bovine SP-D:

| | |
|---|---|
| VNALRQRVGILEGQLQRLQNAFSQYKK | [SEQ ID NO: 102] |

Collectin neck region of rat SP-D:

| | |
|---|---|
| SAALRQQMEALNGKLQRLEAAFSRYK | [SEQ ID NO: 103] |

Collectin neck region of bovine conglutinin:

| | |
|---|---|
| VNALKQRVTILDGHLRRFQNAFSQYKK | [SEQ ID NO: 104] |

Collectin neck region of bovine collectin:

| | |
|---|---|
| VDTLRQRMRNLEGEVQRLQNIVTQYRK | [SEQ ID NO: 105] |

Neck region of human SP-D:

SEQ ID NO: 106
GSPGLKGDKGIPGDKGAKGESGLPDVASLRQQVEALQGQVQHLQAAFSQYK
KVELFPGGIPHRD

Other examples of a MBP trimerizing domain is described in PCT Application Serial No. US08/76266, published as WO 2009/036349, which is incorporated by reference in its entirety. This trimerizing domain can oligomerize even further and create higher order multimeric complexes.

The invention also provides for a general and simple procedure for reliable conversion of an initially selected protein derivative into a final protein product, which without further reformatting may be produced in bacteria (e.g. Escherichia coli) both in small and in large scale (International Patent Application Publication No. WO 94/18227 A2). In certain embodiments, several identical or non-identical binding sites can be included in the same functional protein unit by simple and general means, enabling the exploitation even of weak affinities by means of avidity in the interaction, or the construction of bi- or hetero-functional molecular assemblies (International Patent Application Publication No. WO 98/56906, which is incorporated by reference in its entirety). In certain embodiments, binding can be modulated by the addition or removal of divalent metal ions (e.g. calcium ions) in combinational libraries with one or more preserved metal binding site(s) in the CTLDs. Alternatively, binding can be modulated by altering the pH.

Uses of the CTLD Polypeptides

The combinatorial polypeptide libraries of the invention can be used to generate and identify CTLDs with desired binding properties to target molecules of interest for use in a number of applications including, for example, diagnostic or therapeutic applications in which antibody products are typically used as reagents, in biochemical assay systems, medical in vitro or in vivo diagnostic assay systems, or as active components in therapeutic compositions. The combinatorial polypeptide library comprises altered loop regions that allow for the generation of high affinity binding molecules to selected target moieties.

For use in vitro assay systems, the CTLDs (or CTLD-based protein products) have advantages relative to antibody derivatives as each binding site in a CTLD-based protein product is harbored in a single structurally autonomous protein domain. CTLD domains are resistant to proteolysis, and neither stability nor access to the ligand-binding site is compromised by the attachment of other protein domains to the N- or C-terminus of the CTLD. Accordingly, the CTLD binding module may readily be utilized as a building block for the construction of modular molecular assemblies (e.g., N- and/or C-terminal extensions), for example, harboring multiple CTLDs of identical or non-identical specificity, reporter molecules, enzymatic molecules (peroxidases, phosphatases), effector molecules, radioisotopes, or any other signaling molecule known in the art.

In terms of in vivo use as an essential component of compositions to be used for in vivo diagnostic or therapeutic purposes, the CTLD-based protein products are virtually identical to the corresponding natural CTLD protein already present in the body, and are therefore expected to elicit minimal immunological response in the patient. Single CTLDs are about half the mass of the smallest functional antibody derivative, the single-chain Fv derivative, and this small size may in some applications be advantageous as it may provide better tissue penetration and distribution, as well as a shorter half-life in circulation. Multivalent formats of CTLD proteins, such as those based on the complete tetranectin trimer or the further multimerized collectins, (e.g., mannose binding protein) provide increased binding capacity and avidity and longer circulation half-life.

It should be noted that the section headings are used herein for organizational purposes only, and are not to be construed as in any way limiting the subject matter described. All references cited herein are incorporated by reference in their entirety for all purposes.

The Examples that follow are merely illustrative of certain embodiments of the invention, and are not to be taken as limiting the invention, which is defined by the appended claims.

### EXAMPLES

The vectors discussed in the following Examples (pANA) are derived from vectors that have been previously described [see US 2007/0275393]. Certain vector sequences are provided in the Sequence Listing and one of skill will be able to derive vectors given the description provided herein. The pPhCPAB phage display vector (SEQ ID NO: 50) has the gIII signal peptide coding region has been fused with a linker to the hTN sequence encoding ALQT (etc.). The C-terminal end of the CTLD region is fused via a linker to the remaining gIII coding region. Within the CTLD region, nucleotide mutations were generated that did not alter the coding sequence but generated restriction sites suitable for cloning PCR fragments containing altered loop regions. A portion of the loop region was removed between these restriction sites so that all library phage could only express recombinants and not wild-type tetranectin. The murine TN CTLD phage display vectors are similarly designed. Another embodiment of these vectors is pANA27 (SEQ ID NO: 64) in which the gene III C-terminal region has been truncated and the suppressible stop codon at the end of the hTN coding sequence has been altered to encode glutamine. The murine vector pANA28 (SEQ ID NO: 65) was constructed in a similar fashion.

### Example 1

### Library Construction

### Mutation and Extension of Loop 1

The sequences of human tetranectin and mouse tetranectin, and the positions of loops 1, 2, 3, 4 (LSA) and 5 (LSB) are shown in FIGS. 1, 2 and 4. For the 1-2 extended libraries of human and mouse tetranectin C-type lectin binding domains (“Human 1X-2” and “Mouse 1X-2,” respectively), the coding sequences for Loop 1 were modified to encode the sequences shown in Table 3, where the five amino acids AAEGT (SEQ ID NO: 579; human) or AAEGA (SEQ ID NO: 581; mouse) were substituted with seven random amino acids encoded by the nucleotides NNK NNK NNK NNK NNK NNK NNK (SEQ ID NO: 582); N denotes A, C, G, or T; K denotes G or T. The amino acid arginine immediately following Loop 2 was also fully randomized by using the nucleotides NNK in the coding strand. This amino acid was randomized because the arginine contacts amino acids in Loop 1, and might constrain the configurations attainable by Loop 1 randomization. In addition, the coding sequence for Loop 4 was altered to encode an alanine (A) instead of Lysine 148 (K) in order to abrogate plasminogen binding, which has been shown to be dependent on the Loop 4 lysine (Graversen et al., 1998). The sequences of human tetranectin and mouse tetranectin, and the positions of Loops 1, 2, 3, 4, and 5 are shown in FIG. 2.

TABLE 3
Amino acids of loop regions from human and mouse tetranectin (TN).
Parentheses indicate neighboring amino acids not considered
part of the loop.
X = any amino acid.

| | | Loop 2 | | | |
|---|---|---|---|---|---|
| | Loop 1 | [SEQ ID | Loop 3 | Loop 4 | Loop |
| Library | [SEQ ID NO] | NO] | [SEQ ID NO] | [SEQ ID NO] | 5 |
| Human | DMAAEGTW | DMTGA(R) | NWETEITAQ(P) | DGGKTEN | AAN |
| TN | [107] | [108] | [109] | [110] | |
| Human | DMXXXXXXXW | DMTGA(X) | NWETEITAQ(P) | DGGATEN | AAN |
| 1X-2 | [111] | [112] | [109] | [113] | |
| Human | DMXXXXXW | DMXXX(X) | NWETEITAQ(P) | DGGATEN | AAN |
| 1-2 | [114] | [115] | [109] | [113] | |
| Human | XXXXXXXW | DMTGA(R) | NWETEITAQ(P) | DGGXXXXXEN | AAN |
| 1-4 | [116] | [108] | [109] | [117] | |
| Human | DMAAEGTW | DMTGA(R) | NWXXXXXXQ(P) | DGGATEN | AAN |
| 3X 6 | [107] | [108] | [118] | [113] | |
| Human | DMAAEGTW | DMTGA(R) | NWXXXXXXXQ(P) | DGGATEN | AAN |
| 3X 7 | [107] | [108] | [119] | [113] | |
| Human | DMAAEGTW | DMTGA(R) | NWXXXXXXXXQ(P) | DGGATEN | AAN |
| 3X 8 | [107] | [108] | [120] | [113] | |
| Human | DMAAEGTW | DMTGA(R) | NWETEXXXXXXXTAQ(P) | DGGATEN | AAN |
| 3X loop | [107] | [108] | [121] | [113] | |
| Human | DMAAEGTW | DMTGA(R) | NWETXXXXXXAQ(P) | DGGXXXXXXN | AAN |
| 3-4X | [107] | [108] | [122] | [123] | |
| Human | DMAAEGTW | DMTGA(R) | NWEXXXXXX(X) | XGGXXXN | AAN |
| 3-4 | [107] | [108] | [124] | [125] | |
| combo | | | | | |
| Human | DMAAEGTW | DMTGA(R) | NWEXXXXXQ(P) | DGGATEN | XXX |
| 3-5 | [107] | [108] | [126] | [113] | |
| Human | DMAAEGTW | DMTGA(R) | NWETEITAQ(P) | DGGXXXXXXXN | AAN |
| 4 | [107] | [108] | [109] | [127] | |
| Mouse | DMAAEGAW | DMTGG(L) | NWETEITTQ(P) | DGGKAEN | AAN |
| TN | [128] | [129] | [130] | [131] | |
| Mouse | DMXXXXXXXW | DMTGG(X) | NWETEITTQ(P) | DGGAAEN | AAN |
| 1X-2 | [111] | [132] | [130] | [133] | |
| Mouse | DMXXXXXW | DMXXX(X) | NWETEITTQ(P) | DGGAAEN | AAN |
| 1-2 | [114] | [134] | [130] | [133] | |
| Mouse | XXXXXXXW | DMTGG(L) | NWETEITTQ(P) | DGGXXXXXEN | AAN |
| 1-4 | [116] | [129] | [130] | [117] | |
| Mouse | DMAAEGAW | DMTGG(L) | NWXXXXXXQ(P) | DGGKAEN | AAN |
| 3X | [128] | [129] | [118] | [131] | |
| Mouse | DMAAEGAW | DMTGG(L) | NWXXXXXXXQ(P) | DGGKAEN | AAN |
| 3X | [128] | [129] | [119] | [131] | |
| Mouse | DMAAEGAW | DMTGG(L) | NWXXXXXXXXQ(P) | DGGKAEN | AAN |
| 3X | [128] | [129] | [120] | [131] | |
| Mouse | DMAAEGAW | DMTGG(L) | NWETEXXXXXXXTTQ(P) | DGGKAEN | AAN |
| 3X loop | [128] | [129] | [135] | [131] | |
| Mouse | DMAAEGAW | DMTGG(L) | NWETXXXXXXTQ(P) | DGGXXXXXXN | AAN |
| 3-4X | [128] | [129] | [136] | [123] | |
| Mouse | DMAAEGAW | DMTGG(L) | NWEXXXXXX(X) | XGGXXXN | AAN |
| 3-4 | [128] | [129] | [124] | [125] | |
| combo | | | | | |
| Mouse | DMAAEGAW | DMTGG(L) | NWEXXXXXQ(P) | DGGKAEN | XXX |
| 3-5 | [128] | [129] | [126] | [131] | |
| Mouse | DMAAEGAW | DMTGG(L) | NWETEITTQ(P) | DGGXXXXXXXN | AAN |
| 4 | [128] | [129] | [130] | [127] | |

The human Loop 1 extended library was generated using overlap PCR in the following manner (primer sequences are shown in Table 4). Primers 1Xfor (SEQ ID NO: 137) and 1Xrev (SEQ ID NO: 138) were mixed and extended by PCR, and primers BstX1for (SEQ ID NO: 139) and PstBssRevC (SEQ ID NO: 140) were mixed and extended by PCR. The resulting fragments were purified from gels, and mixed and extended by PCR in the presence of the outer primers Bglfor12 (SEQ ID NO: 141) and PstRev (SEQ ID NO: 142). The resulting fragment was gel purified and cut with Bgl II and Pst I and cloned into a phage display vector pPhCPAB or pANA27. The phage display vector pPhCPAB was derived from pCANTAB (Pharmacia), and contained a portion of the human tetranectin CTLD fused to the M13 gene III protein. The CTLD region was modified to include Bgl II and Pst I restriction enzyme sites flanking Loops 1-4, and the 1-4 region was altered to include stop codons, such that no functional gene III protein could be produced from the vector without ligation of an in-frame insert. pANA27 was derived from pPhCPAB by replacing the BamHI to ClaI regions with the BamHI to ClaI sequence of SEQ ID NO:64 (pANA27). This replaces the amber suppressible stop codon with a glutamine codon and truncates the amino terminal region of gene III.

Ligated material was transformed into electrocompetent XL1-Blue E. coli (Stratagene) and four to eight liters of cells were grown overnight and DNA isolated to generate a master library DNA stock for panning A library size of 1.5×10⁸ was obtained, and clones examined showed diversified sequence in the targeted regions.

The mouse Loop 1 extended library was generated using overlap PCR in the following manner. Primers Mu1Xfor (SEQ ID NO: 143) and Mu1Xrev (SEQ ID NO: 144) were mixed and extended by PCR, and primers Mu1XSal1 for (SEQ ID NO: 145) and Mu1XPstRev (SEQ ID NO: 146) were mixed and extended by PCR. The resulting fragments were purified from gels, mixed and extended by PCR in the presence of the outer primers BstBBssH (SEQ ID NO: 147) and Mu Pst (SEQ ID NO: 148). The resulting fragment was gel purified and cut with BssH II and Pst I and ligated into similarly digested phage display vector pANA16 or pANA28. Phage display vector pANA16 (SEQ ID NO: 63) was derived from pPhCPAB by replacing the human tetranectin CTLD with the mouse tetranectin CTLD. The mouse tetranectin CTLD included BstBI, BssHII, and SalI sites within the Loop 1-4 region and a PstI site after the Loop 4 region similar to pPhCPAB in order to facilitate cloning. In addition, the region was altered to include stop codons as described above. Phage display vector pANA28 (SEQ ID NO:65) was derived from pANA16 (SEQ ID NO:63) by replacing the BamHI to ClaI region with the BamHI to ClaI sequence given in SEQ ID NO:65. Ligated material was transformed into electrocompetent XL1-Blue E. coli (Stratagene) and four to eight liters of cells were grown overnight and DNA isolated to generate a master library DNA stock for panning. A library size of 2.65×10¹⁰ was obtained, and clones examined showed diversified sequence in the targeted regions.

TABLE 4
Sequences used in the generation of phage displayed C-type lectin domain
libraries.
M = A or C; N = A, C, G, or T; K = G or T; S = G or C; W = A or T.

| | | SEQ ID |
|---|---|---|
| Name | Sequence | NO |
| 1Xfor | GGCTGGGCCT GAACGACATG NNKNNKNNKN NKNNKNNKNN KTGGGTGGAT | 137 |
| | ATGACTGGCG CC | |
| 1Xrev | GGCGGTGATC TCAGTTTCCC AGTTCTTGTA GGCGATMNNG GCGCCAGTCA | 138 |
| | TATCCACCCA | |
| BstX1for | ACTGGGAAAC TGAGATCACC GCCCAACCTG ATGGCGGCGC AACCGAGAAC | 139 |
| | TGCGCGGTCC TG | |
| PstBssRev | CCCTGCAGCG CTTGTCGAAC CACTTGCCGT TGGCGGCGCC AGACAGGACC | 140 |
| C | GCGCAGTTCT | |
| Bg1for12 | GCCGAGATCT GGCTGGGCCT GAACGACATG | 141 |
| PstRev | ATCCCTGCAG CGCTTGTCGA ACC | 142 |
| Mu1Xfor | GCTGTTCGAA TACGCGCGCC ACAGCGTGGG CAACGATGCG AACATCTGGC | 143 |
| | TGGGCCTCAA CGATATG | |
| Mu1Xrev | GCCGCCGGTC ATGTCGACCC AMNNMNNMNN MNNMNNMNNM NNCATATCGT | 144 |
| | TGAGGCCCAG CCAG | |
| Mu1XSalFo | TGGGTCGACA TGACCGGCGG CNNKCTGGCC TACAAGAACT GGGAGACGGA | 145 |
| r | GATCACGACG CAACCCGACG GCGGCGCTGC CGAGAACTG | |
| Mu1XPstRe | CAGCGTTTGT CGAACCACTT GCCGTTGGCT GCGCCAGACA GGGCGGCGCA | 146 |
| v | GTTCTCGGCA GCGCCGCCGT CGGGTT | |
| BstBBssH | GCTGTTCGAA TACGCGCGCC ACAGCGTGG | 147 |
| Mu Pst | GGGCAACTGA TCTCTGCAGC GTTTGTCGAA CCACTTGCCG T | 148 |
| 1-2 for | GGCTGGGCCT GAACGACATG NNKNNKNNKN NKNNKTGGGT GGATATGNNK | 149 |
| | NNKNNKNNKA TCGCCTACAA GAACTGGGA | |
| 1-2 rev | GACAGGACGG CGCAGTTCTC GGTTGCGCCG CCATCAGGTT GGGCGGTGAT | 150 |
| | CTCAGTTTCC CAGTTCTTGT AGGCGAT | |
| PstRev12 | ATCCCTGCAG CGCTTGTCGA ACCACTTGCC GTTGGCGGCG CCAGACAGGA | 151 |
| | CGGCGCAGTT CTC | |
| Mu12rev | CGTCTCCCAG TTCTTGTAGG CCAGMNNMNN MNNMNNCATG TCGACCCAMN | 152 |
| | NMNNMNNMNN MNNCATATCG TTGAGGCCCA GCCAG | |
| Mu1234for | GCCTACAAGA ACTGGGAGAC GGAGATCACG ACGCAACCCG ACGGCGGCGC | 153 |
| | TGCCGAGAAC TG | |
| Bg1Bssfor | GAGATCTGGC TGGGCCTCAA CNNSNNSNNS NNSNNSNNSN NSTGGGTGGA | 154 |
| | CATGACTGGC | |
| BssBg1rev | TTGCGCGGTG ATCTCAGTCT CCCAGTTCTT GTAGGCGATA CGCGCGCCAG | 155 |
| | TCATGTCCAC CCA | |
| BssPstfor | GACTGAGATC ACCGCGCAAC CCGATGGCGG CNNSNNSNNS NNSNNSGAGA | 156 |
| | ACTGCGCGGT CCTG | |
| PstBssRev | CCCTGCAGCG CTTGTCGAAC CACTTGCCGT TGGCCGCGCC TGACAGGACC | 157 |
| | GCGCAGTTCT | |
| Bg1for | GCCGAGATCT GGCTGGGCCT CA | 158 |
| MuUpsF | GCCATGGCCG CCTTACAGAC TGTGTGCCTG AAG | 159 |
| MuRanR | CGTCTCCCAG TTCTTGTAGG CCAGGAGGCC GCCGGTCATG TCCACCCAMN | 160 |
| | NMNNMNNMNN MNNMNNMNNG TTGAGGCCCA GCCAGAT | |
| MuRanF | GCCTACAAGA ACTGGGAGAC GGAGATCACG ACGCAACCCG ACGGCGGCNN | 161 |
| | KNNKNNKNNK NNKGAGAACT GCGCCGCCCT G | |
| MuDnsR | CGCACCTGCG GCCGCCACAA TGGCAAACTG GCAGATGT | 162 |
| H Loop 1- | ATCTGGCTGG GCCTGAACGA CATGGCCGCC GAGGGCACCT GGGTGGATAT | 163 |
| 2-F | GACCGGCGCG CGTATCGCCT ACAAGAAC | |
| H Loop 3- | CCGCCATCGG GTTGGGCMNN MNNMNNMNNM NNMNNAGTTT CCCAGTTCTT | 164 |
| 4 Ext R | GTAGGCGATA CG | |
| H Loop 3- | GCCCAACCCG ATGGCGGCNN KNNKNNKNNK NNKNNKAACT GCGCCGTCCT | 165 |
| 4 Ext-F | GTCTGGC | |
| H Loop 5- | CCTGCAGCGC TTGTCGAACC ACTTGCCGTT GGCGGCGCCA GACAGGACGG | 166 |
| R | CGCA | |
| M SacII-F | GACATGGCCG CGGAAGGCGC CTGGGTCGAC ATGACCGGCG GCCTGCTGGC | 167 |
| | CTACAAGAAC | |
| M Loop 3- | CCGCCGTCGG GTTGGGTMNN MNNMNNMNNM NNMNNGGTCT CCCAGTTCTT | 168 |
| 4 Ext-R | GTAGGCCAGC A | |
| M Loop 3- | ACCCAACCCG ACGGCGGCNN KNNKNNKNNK NNKNNKAACT GCGCCGCCCT | 169 |
| 4 Ext-F | GTCTGGC | |
| M Loop 5- | CTGATCTCTG CAGCGCTTGT CGAACCACTT GCCGTTGGCT GCGCCAGACA | 170 |
| R | GGGCGGCGCA GTT | |
| H Loop 3- | GCCAGACAGG ACGGCGCAGT TMNNMNNMNN GCCGCCMNNM NNMNNMNNMN | 171 |
| 4 Combo R | NMNNMNNMNN TTCCCAGTTC TTGTAGGCGA TACG | |
| M Loop 3- | GCCAGACAGG GCGGCGCAGT TMNNMNNMNN GCCGCCMNNM NNMNNMNNMN | 172 |
| 4 Combo R | NMNNMNNMNN CTCCCAGTTC TTGTAGGCCA GCA | |
| H Loop 3- | CCGCCATCGG GTTGGGCGGT GATCTCAGTT TCCCAGTTCT TGTAGGCGAT | 173 |
| R | ACG | |
| H Loop 4 | GCCCAACCCG ATGGCGGCNN KNNKNNKNNK NNKNNKNNKA ACTGCGCCGT | 174 |
| Ext-F | CCTGTCTGGC | |
| M Loop 3- | CCGCCGTCGG GTTGGGTGGT GATCTCGGTC TCCCAGTTCT TGTAGGCCAG | 175 |
| R | CA | |
| M Loop 4 | ACCCAACCCG ACGGCGGCNN KNNKNNKNNK NNKNNKNNKA ACTGCGCCGC | 176 |
| Ext-F | CCTGTCTGGC | |
| HLoop3F 6 | CTGGCGCGCG TATCGCCTAC AAGAACTGGN NKNNKNNKNN KNNKNNKCAA | 177 |
| | CCCGATGGCG GCGCCACCGA GAAC | |
| HLoop3F 7 | CTGGCGCGCG TATCGCCTAC AAGAACTGGN NKNNKNNKNN KNNKNNKNNK | 178 |
| | CAACCCGATG GCGGCGCCAC CGAGAAC | |
| HLoop3F 8 | CTGGCGCGCG TATCGCCTAC AAGAACTGGN NKNNKNNKNN KNNKNNKNNK | 179 |
| | CAACCCGATG GCGGCGCCAC CGAGAAC | |
| HLoop4R | CCTGCAGCGC TTGTCGAACC ACTTGCCGTT GGCGGCGCCA GACAGGACGG | 180 |
| | CGCAGTTCTC GGTGGCGCCG CCATCGGGTT G | |
| MLoop3F 6 | GTTCTCGGCA GCGCCGCCGT CGGGTTGMNN MNNMNNMNNM NNMNNCCAGT | 181 |
| | TCTTGTAGGC CAGCAGGCCG CCGGTCA | |
| MLoop3F 7 | GTTCTCGGCA GCGCCGCCGT CGGGTTGMNN MNNMNNMNNM NNMNNMNNCC | 182 |
| | AGTTCTTGTA GGCCAGCAGG CCGCCGGTCA | |
| MLoop3F 8 | GTTCTCGGCA GCGCCGCCGT CGGGTTGMNN MNNMNNMNNM NNMNNMNNMN | 183 |
| | NCCAGTTCTT GTAGGCCAGC AGGCCGCCGG TCA | |
| M 3X OF | GACATGGCCGCGGAAGGC | 184 |
| H1-3-4R | GACAGGACCG CGCAGTTCTC GCCSMAGWMC CCSAAGCCGC CMNNGGGTTG | 185 |
| | MNNMNNMNNM NNMNNCTCCC AGTTCTTGTA GGCGATACG | |
| PstLoop4 | ATCCCTGCAG CGCTTGTCGA ACCACTTGCC GTTGGCCGCG CCTGACAGGA | 186 |
| rev | CCGCGCAGTT CTCGCC | |
| Loop3AF2 | GAGCGTGGGCAACGAGGCCGAGATCTGGCTGGGCCTCAACGACATGGCCGCCGA | 187 |
| Loop3AR2 | CCAGTTCTTGTAGGCGATACGCGCGCCAGTCATATCCACCCAGGTGCCCTCGGC | 188 |
| | GGCCATGTCGTTGAGG | |
| Loop3BF | ATCGCCTACAAGAACTGGGAGACTGRGNNKNNKNNKNNKNNKNNKNNKACCGCG | 189 |
| | CAACCCGATGGCGGTGCAAC | |
| Loop3BR | CGCTTGTCGAACCACTTGCCGTTGGCGGCGCCAGACAGGACGGCGCAGTTCTCG | 190 |
| | GTTGCACCGCCATCGGGTTG | |
| Loop3OR | GATCCCTGCAGCGCTTGTCGAACCACTTGCCGT | 191 |
| M 3X OR | GCAGATGTAGGGCAACTGATCTCT | 192 |
| HuBg1for | GCCGAGATCTGGCTGGGCCTGA | 193 |
| GSXX | GCCGAGATCTGGCTGGGCCTCAACGGCAGCNNKNNKNNKNNKWCCTGGGTGGAC | 194 |
| | ATGACTGGC | |
| 090827 | TTGCGCGGTGATCTCAGTCTCCCAGTTCTTGTAGGCGATACGCGCGCCAGTCAT | 195 |
| BssBg1rev | GTCCACCCA | |
| FGVFGfor | GACTGAGATCACCGCGCAACCCGATGGCGGCTTCGGCGTGTTCGGCGAGAACTG | 196 |
| | CGCGGTCCTG | |
| WGVFGfor | GACTGAGATCACCGCGCAACCCGATGGCGGCTGGGGCGTGTTCGGCGAGAACTG | 197 |
| | CGCGGTCCTG | |
| FGYFGfor | GACTGAGATCACCGCGCAACCCGATGGCGGCTTCGGGTACTTCGGCGAGAACTG | 198 |
| | CGCGGTCCTG | |
| WGYFGfor | GACTGAGATCACCGCGCAACCCGATGGCGGCTGGGGGTACTTCGGCGAGAACTG | 199 |
| | CGCGGTCCTG | |
| WGVWGfor | GACTGAGATCACCGCGCAACCCGATGGCGGCTGGGGCGTGTGGGGCGAGAACTG | 200 |
| | CGCGGTCCTG | |
| Mu 1-4 AF | GGCAACGATGCGAACATCTGGCTGGGCCTCAACNNKNNKNNKNNKNNKNNKNNK | 201 |
| | TGGGTCGACATGACCGGC | |
| Mu 1-4 AR | GGTTGCGTCGTGATCTCCGTCTCCCAGTTCTTGTAGGCCAGGAGGCCGCCGGTC | 202 |
| | ATGTCGACCCA | |
| Mu 1-4 BF | GACGGAGATCACGACGCAACCCGACGGCGGCNNKNNKNNKNNKNNKGAGAACTG | 203 |
| | TGCTGCCCTGTCTGG | |
| Mu 1-4 BR | CTCTGCAGCGCTTGTCGAACCACTTGCCGTTGGCTGCGCCAGACAGGGCAGCAC | 204 |
| | AGTTCTC | |
| Mu 1-4 OF | ATACGCGCGCCACAGCGTGGGCAACGATGCGAACATCTG | 205 |
| Mu 1-4 OR | ATCTCTGCAGCGCTTGTCGAACC | 206 |
| Mloop4F | CAACCCGACGGCGGCGCTGCCGAGAACTGCGCCGCCCTGTCTGGCGCAGCCAAC | 207 |
| | GGCAAGTG | |
| M MfeR | GCAGATGTAGGGCAACTGATCTCTGCAGCGCTTGTCGAACCACTTGCCGTTGGC | 208 |
| | TGCGCCAGAC | |
| m3-5 for | GCTGGCCTACAAGAACTGGGAGNNKNNKNNKNNKNNKCAACCCGACGGCGGCGC | 209 |
| | AGCTGAGAACTG | |
| m3-5 rev | GCGCTTGTCGAACCACTTGCCMNNMNNMNNGCCAGACAGGGCGGCGCAGTTCTC | 210 |
| | AGCTGCGCCGCCGT | |
| m3-5 OF | CTGGGTCGACATGACCGGCGGCCTGCTGGCCTACAAGAACTGGGAG | 211 |
| m3-5 OR | ATCTCTGCAGCGCTTGTCGAACCACTTG | 212 |
| h3-5AF | TGGGCCTGAACGACATGGCCGCCGAGGGCACCTGGGTGGATATGACTGGCGCGC | 213 |
| | GTATCGCCTACAAGAACTGGGAG | |
| h3-5AR | GTTGCGCCGCCATCGGGTTGMNNMNNMNNMNNMNNCTCCCAGTTCTTGTAGGCG | 214 |
| | ATACG | |
| h3-5BF | CAACCCGATGGCGGCGCAACCGAGAACTGCGCCGTCCTGTCTGG | 215 |
| h3-5BR | TGTAGGGCAATTGATCCCTGCAGCGCTTGTCGAACCACTTGCCMNNMNNMNNGC | 216 |
| | CAGACAGGACGGCGCAGTT | |
| h3-5 OF | GCCGAGATCTGGCTGGGCCTGAACGACATGG | 217 |

### Example 2

### Library Construction

### Mutation of Loops 1 and 2

For the Loop 1-2 libraries of human and mouse tetranectin C-type lectin binding domains (“Human 1-2” and “Mouse 1-2,” respectively), the coding sequences for Loop 1 were modified to encode the sequences shown in Table 1, where the five amino acids AAEGT (SEQ ID NO: 579; human) or AAEGA (SEQ ID NO: 581; mouse) were replaced with five random amino acids encoded by the nucleotides NNK NNK NNK NNK NNK (SEQ ID NO: 583); N denotes A, C, G, or T; K denotes G or T). In Loop 2 (including the neighboring arginine), the four amino acids TGAR (SEQ ID NO: 584) in human or TGGR (SEQ ID NO: 585) in mouse were replaced with four random amino acids encoded by the nucleotides NNK NNK NNK NNK (SEQ ID NO: 586). In addition, the coding sequence for Loop 4 was altered to encode an alanine (A) instead of the lysine (K) in the loop, in order to abrogate plasminogen binding, which has been shown to be dependent on the Loop 4 lysine (Graversen et al., 1998).

The human 1-2 library was generated using overlap PCR in the following manner (primer sequences are shown in Table 4). Primers 1-2 for (SEQ ID NO: 149) and 1-2 rev (SEQ ID NO: 150) were mixed and extended by PCR. The resulting fragment was purified from gels, mixed and extended by PCR in the presence of the outer primers Bglfor12 (SEQ ID NO: 141) and PstRev12 (SEQ ID NO: 151). The resulting fragment was gel purified and cut with Bgl II and Pst I and cloned into similarly digested phage display vector pPhCPAB or pANA27, as described above. A library size of 4.86×10⁸ was obtained, and clones examined showed diversified sequence in the targeted regions.

The mouse Loop 1-2 library was generated using overlap PCR in the following manner. Primers Mu1Xfor (SEQ ID NO: 143) and Mu12rev (SEQ ID NO: 152) were mixed and extended by PCR, and primers Mu1234for (SEQ ID NO: 153) and Mu1XPstRev (SEQ ID NO: 146) were mixed and extended by PCR. The resulting fragments were purified from gels, mixed and extended by PCR in the presence of the outer primers BstBBssH (SEQ ID NO: 147) and Mu Pst (SEQ ID NO: 148). The resulting fragment was gel purified and cut with BssH II and Pst I and cloned into similarly digested phage display vector pANA16 or pANA28, as described above. A library size of 1.63×10⁹ was obtained, and clones examined showed diversified sequence in the targeted regions.

### Example 3

### Library Construction

### Mutation and Extension of Loops 1 and 4

For the Loop 1-4 libraries of human and mouse tetranectin C-type lectin binding domains (“Human 1-4” and “Mouse 1-4,” respectively), the coding sequences for Loop 1 were modified to encode the sequences shown in Table 3, where the seven amino acids DMAAEGT (see SEQ ID NO: 587; human) or DMAAEGA (see SEQ ID NO: 588; mouse) were replaced with seven random amino acids encoded by the nucleotides NNK NNK NNK NNK NNK NNK NNK (SEQ ID NO: 582); N denotes A, C, G, or T; K denotes G or T). In Loop 4 two amino acids KT in human or KA in mouse, were replaced with five random amino acids encoded by the nucleotides NNK NNK NNK NNK NNK (SEQ ID NO: 583).

The human 1-4 library was generated using overlap PCR in the following manner (primer sequences are shown in Table 4). Primers BglBssfor (SEQ ID NO: 154) and BssBglrev (SEQ ID NO: 155) were mixed and extended by PCR, and primers BssPstfor (SEQ ID NO: 156) and PstBssRev (SEQ ID NO: 157) were mixed and extended by PCR. The resulting fragments were purified from gels, mixed and extended by PCR in the presence of the outer primers Bglfor (SEQ ID NO: 158) and PstRev (SEQ ID NO: 142). The resulting fragment was gel purified and cut with Bgl II and Pst I restriction enzymes, and cloned into similarly digested phage display vector pPhCPAB or pANA27, as described above. A library size of 2×10⁹ was obtained, and 12 clones examined prior to panning showed diversified sequence in the targeted regions.

The mouse 1-4 library was generated using overlap PCR in the following manner (primer sequences are shown in Table 4). Primers Mu 1-4 AF (SEQ ID NO: 201) and Mu 1-4 AR (SEQ ID NO: 202) were mixed and extended by PCR, and primers Mu 1-4 BF (SEQ ID NO: 203) and Mu 1-4 BR (SEQ ID NO: 204) were mixed and extended by PCR. The resulting fragments were purified from gels, mixed and extended by PCR in the presence of the outer primers Mu 1-4 OF (SEQ ID NO: 205) and Mu 1-4 OR (SEQ ID NO: 206). The resulting fragment was gel purified and cut with BstB I and Pst I restriction enzymes, and cloned into similarly digested phage display vector pANA28, as described above. A library size of 4.7×10⁹ was obtained, and >20 clones were examined prior to panning showed diversified sequence in the targeted regions.

### Example 4

### Library Construction

### Mutation and Extension of Loops 3 and 4

For the Loop 3-4 extended libraries of human and mouse tetranectin C-type lectin binding domains (“Human 3-4X” and “Mouse 3-4X,” respectively), the coding sequences for Loop 3 were modified to encode the sequences shown in Table 4, where the three amino acids EIT of human or mouse tetranectin were replaced with six random amino acids encoded by the nucleotides NNK NNK NNK NNK NNK NNK (SEQ ID NO: 589) in the coding strand (N denotes A, C, G, or T; K denotes G or T). In addition, in Loop 4, the three amino acids KTE in human or KAE in mouse were replaced with six random amino acids encoded by the nucleotides NNK NNK NNK NNK NNK NNK (SEQ ID NO: 589).

The human 3-4 extended library was generated using overlap PCR in the following manner (primer sequences are shown in Table 4). Primers H Loop 1-2-F (SEQ ID NO: 163) and H Loop 3-4 Ext-R (SEQ ID NO: 164) were mixed and extended by PCR, and primers H Loop 3-4 Ext-F (SEQ ID NO: 165) and H Loop 5-R (SEQ ID NO: 166) were mixed and extended by PCR. The resulting fragments were purified from gels, and mixed and extended by PCR in the presence of additional H Loop 1-2-F (SEQ ID NO: 163) and H Loop 5-R (SEQ ID NO: 166). The resulting fragment was gel purified and cut with Bgl II and Pst I restriction enzymes, and cloned into similarly digested phage display vector pPhCPAB or pANA27, as described above. A library size of 7.9×10⁸ was obtained, and clones examined showed diversified sequence in the targeted regions.

The mouse 3-4 extended library was generated using overlap PCR in the following manner. Primers M SacII-F (SEQ ID NO: 167) and M Loop 3-4 Ext-R (SEQ ID NO: 168) were mixed and extended by PCR, and primers M Loop 3-4 Ext-F (SEQ ID NO: 169) and M Loop 5-R (SEQ ID NO: 170) were mixed and extended by PCR. The resulting fragments were purified from gels, and mixed and extended by PCR in the presence of additional M SacII-F (SEQ ID NO: 167) and M Loop 5-R (SEQ ID NO: 170). The resulting fragment was gel purified and cut with Sac II and Pst I restriction enzymes, and cloned into similarly digested phage display vector pANA16 or pANA28, as described above. A library size of 4.95×10⁹ was obtained, and clones examined showed diversified sequence in the targeted regions.

### Example 5

### Library Construction

### Mutation of Loops 3 and 4 and the PRO Between the Loops

For the Loop 3-4 combo library of human and mouse tetranectin C-type lectin binding domains (“Human 3-4 combo” and “Mouse 3-4 combo,” respectively), the coding sequences for loops 3 and 4 and the proline between these two loops were altered to encode the sequences shown in Table 3, where the human sequence TEITAQPDGGKTE (SEQ ID NO: 590) or the corresponding mouse sequence TEITTQPDGGKAE (SEQ ID NO: 591) were replaced by the 13 amino acid sequence XXXXXXXXGGXXX, (SEQ ID NO: 592) where X represents a random amino acid encoded by the sequence NNK (N denotes A, C, G, or T; K denotes G or T).

The human 3-4 combo library was generated using overlap PCR in the following manner (primer sequences are shown in Table 4). Primers H Loop 1-2-F (SEQ ID NO: 163) and H Loop 3-4 Combo-R (SEQ ID NO: 171) were mixed and extended by PCR and the resulting fragment was purified from gels and mixed and extended by PCR in the presence of additional H Loop 1-2-F (SEQ ID NO: 163) and H loop 5-R (SEQ ID NO: 166). The resulting fragment was gel purified and cut with Bgl II and Pst I restriction enzymes, and cloned into similarly digested phage display vector pPhCPAB or pANA27, as described above. A library size of 4.95×10⁹ was obtained, and clones examined showed diversified sequence in the targeted regions.

The mouse 3-4 combo library was generated using overlap PCR in the following manner. Primers M SacII-F (SEQ ID NO: 167) and M Loop 3-4 Combo-R (SEQ ID NO: 172) were mixed and extended by PCR and the resulting fragment was purified from gels and mixed and extended by PCR in the presence of the outer primers M SacII-F (SEQ ID NO: 167) and M Loop 5-R (SEQ ID NO: 170). The resulting fragment was gel purified and cut with Sac II and Pst I restriction enzymes, and cloned into similarly digested phage display vector pANA16 or pANA28, as described above. A library size of 7.29×10⁸ was obtained, and clones examined showed diversified sequence in the targeted regions.

### Example 6

### Library Construction

### Mutation and Extension of Loop 4

For the Loop 4 extended libraries of human and mouse tetranectin C-type lectin binding domains (“Human 4” and “Mouse 4,” respectively), the coding sequences for Loop 4 were modified to encode the sequences shown in Table 3, where the three amino acids KTE of human or KAE of mouse tetranectin were replaced with seven random amino acids encoded by the nucleotides NNK NNK NNK NNK NNK NNK NNK (SEQ ID NO: 582); N denotes A, C, G, or T; K denotes G or T).

The human 4 extended library was generated using overlap PCR in the following manner (primer sequences are shown in Table 4). Primers H Loop 1-2-F (SEQ ID NO: 163) and H Loop 3-R (SEQ ID NO: 173) were mixed and extended by PCR, and primers H Loop 4 Ext-F (SEQ ID NO: 174) and H Loop 5-R (SEQ ID NO: 166) were mixed and extended by PCR. The resulting fragments were purified from gels, and mixed and extended by PCR in the presence of additional H Loop 1-2-F (SEQ ID NO: 163) and H Loop 5-R (SEQ ID NO: 166). The resulting fragment gel purified and was cut with Bgl II and Pst I restriction enzymes, and cloned into similarly digested phage display vector pPhCPAB or pANA27, as described above. A library size of 2.7×10⁹ was obtained, and clones examined showed diversified sequence in the targeted regions.

The mouse 4 extended library was generated using overlap PCR in the following manner. Primers M SacII-F (SEQ ID NO: 167) and M Loop 3-R (SEQ ID NO: 175) were mixed and extended by PCR, and primers M Loop 4 Ext-F (SEQ ID NO: 176) and M Loop 5-R (SEQ ID NO: 170) were mixed and extended by PCR. The resulting fragments were purified from gels, and mixed and extended by PCR in the presence of the additional M SacII-F (SEQ ID NO: 167) and M Loop 5-R (SEQ ID NO: 170). The resulting fragment was gel purified, digested with SacII and PstI restriction enzymes, and cloned into similarly digested phage display vector pANA16 or pANA28, as described above.

### Example 7

### Library Construction

### Mutation with and without Extension of Loop 3

For the Loop 3 altered libraries of human and mouse tetranectin C-type lectin binding domains, the coding sequences for Loop 3 were modified to encode the sequences shown in Table 3, where the six amino acids ETEITA (SEQ ID NO: 593) of human or ETEITT (SEQ ID NO: 594) of mouse tetranectin were replaced with six, seven, or eight random amino acids encoded by the nucleotides NNK NNK NNK NNK NNK NNK (SEQ ID NO: 583), NNK NNK NNK NNK NNK NNK NNK (SEQ ID NO: 582), and NNK NNK NNK NNK NNK NNK NNK NNK (SEQ ID NO: 595); N denotes A, C, G, or T; and K denotes G or T. In addition, in Loop 4, the three amino acids KTE in human or KAE in mouse were replaced with six random amino acids encoded by the nucleotides NNK NNK NNK NNK NNK NNK (SEQ ID NO: 589). In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

The human Loop 3 altered library was generated using overlap PCR in the following manner. Primers HLoop3F6, HLoop3F7, and HLoop3F8 (SEQ ID NOS: 177-179, respectively) were individually mixed with HLoop4R (SEQ ID NO: 180) and extended by PCR. The resulting fragments were purified from gels, and mixed and extended by PCR in the presence of oligos H Loop 1-2F (SEQ ID NO: 163), HuBglfor (SEQ ID NO: 193) and PstRev (SEQ ID NO: 142). The resulting fragments were gel purified, digested with BglI and PstI restriction enzymes, and cloned into similarly digested phage display vector pPhCPAB or pANA27, as above. After library generation, the three libraries were pooled for panning.

The mouse Loop 3 altered library was generated using overlap PCR in the following manner. Primers MLoop3F 6, MLoop3F 7, and MLoop3F 8 (SEQ ID NOS: 181-183, respectively) were individually mixed with primer M SacII-F (SEQ ID NO: 167) and extended by PCR. In addition, primers MLoop4F (SEQ ID NO: 207) and M MfeR (SEQ ID NO: 208) were mixed and extended by PCR. The resulting fragments were purified from gels, mixed, and subjected to PCR in the presence of primers M 3X OF (SEQ ID NO: 184) and M 3X OR (SEQ ID NO: 192). Products were digested with Sal I (or Sac II) and PstI restriction enzymes, and the purified fragments were cloned into similarly digested phage display vector pANA16 or pANA28, as described above.

Alternate Loop Extension of Loop 3

The human loop 3 loop library was generated using overlap PCR in the following manner. Primers Loop3AF2 (SEQ ID NO: 187) and Loop3AR2 (SEQ ID NO: 188) are mixed and extended by PCR, and primers Loop3BF (SEQ ID NO: 189) and Loop3BR (SEQ ID NO: 190) are mixed and extended by PCR. The resulting fragments are purified from gels, mixed, and subjected to PCR in the presence of primers Bglfor (SEQ ID NO: 158) and Loop3OR (SEQ ID NO: 191). Products are digested with Bgl II and Pst I restriction enzymes, and the purified fragments are cloned into similarly digested phage display vector pPhCPAB or pANA27, as above. In addition the coding sequence for loop 4 was altered to encode an alanine (A) instead of the lysine (K) in the loop, in order to abrogate plasminogen binding, which has been shown to be dependent on the loop 4 lysine (Graversen et al., 1998). A similar approach can be used to generate the corresponding mouse TN library.

### Example 8

### Mutation of Loops 3 and 5

For the loop 3 and 5 altered libraries of human and mouse tetranectin C-type lectin binding domains, the coding sequences for loops 3 and 5 were modified to encode the sequences shown in Table 3, where the five amino acids TEITA (SEQ ID NO: 596) of human or TEITT (SEQ ID NO: 597) of mouse tetranectin were replaced with five amino acids encoded by the nucleotides NNK NNK NNK NNK NNK (SEQ ID NO: 583), and the three Loop 5 amino acids AAN of human or mouse were replaced with three amino acids encoded by the nucleotides NNK NNK NNK. In addition the coding sequence for loop 4 was altered to encode an alanine (A) instead of the lysine (K) in the loop, in order to abrogate plasminogen binding, which has been shown to be dependent on the loop 4 lysine (Graversen et al., 1998).

The human loop 3 and 5 altered library was generated using overlap PCR in the following manner. Primers h3-5AF (SEQ ID NO: 213) and h3-5AR (SEQ ID NO: 214) were mixed and extended by PCR, and primers h3-5BF (SEQ ID NO: 215) and h3-5 BR (SEQ ID NO: 216) were mixed and extended by PCR. The resulting fragments were purified from gels, and mixed and extended by PCR in the presence of h3-5 OF (SEQ ID NO: 217) and PstRev (SEQ ID NO: 142). The resulting fragment was gel purified, digested with Bgl I and Pst I restriction enzymes, and cloned into similarly digested phage display vector pPhCPAB or pANA27 as described above.

The mouse loop 3 and 5 altered library was generated using overlap PCR in the following manner. Primers m3-5 for (SEQ ID NO: 209) and m3-5 rev (SEQ ID NO: 210) were mixed and extended by PCR. The resulting fragment was purified from gels, and reamplified by PCR with primers m3-5OF (SEQ ID NO: 211) and m3-5 OR (SEQ ID NO: 212). Products were digested with Sal I and Pst I restriction enzymes, and the purified fragments were cloned into similarly digested phage display vector pANA16 or pANA28 as described above.

Examples 9-22 provide exemplary methods for isolating polypeptide sequences specific for TRAIL death receptors using the combinatorial polypeptide libraries of the invention. TRAIL (tumor necrosis factor-related apoptosis-inducing ligand, also referred to in the literature as Apo2L and TNFSF10, among other things) belongs to the tumor necrosis factor (TNF) superfamily and has been identified as an activator of programmed cell death, or apoptosis, in tumor cells. TRAIL is expressed in cells of the immune system including NK cells, T cells, macrophages, and dendritic cells and is located in the cell membrane. TRAIL can be processed by cysteine proteases, generating a soluble form of the protein. Both the membrane-bound and soluble forms of TRAIL function as trimers and are able to trigger apoptosis via interaction with TRAIL receptors located on target cells. In humans, five receptors have been identified to have binding activity for TRAIL. Two of these five receptors, TRAIL-R1 (DR 4, TNFSF10a) and TRAIL-R2 (DR 5, TNFRSF10b), contain a cytoplasmic region called the death domain (DD). The death domain on these two receptor molecules is required for TRAIL-activation of the extrinsic apoptotic pathway upon the binding of TRAIL to the receptors. The remaining three TRAIL receptors (called TRAIL-R3 (DcR1, TNFRSF10c), TRAIL-R4 (DcR2, TNFRSF10d) and circulating osteoprotegerin (OPG, TNFRSF11b)) are thought to serve as decoy receptors. These three receptors lack functional DDs and are thought to be mainly involved in negatively regulating apoptosis by sequestering TRAIL or stimulating pro-survival signals.

Upon binding of TRAIL to TRAIL-R1 (DR 4) or -R2 (DR 5) the trimerized receptors recruit several cytosolic proteins that form the death-inducing signaling complex (DISC) which subsequently leads to activation of caspase-8 or caspase-10. This triggers one of two different routes that cause irreversible cell death, one in which caspase-8 directly activates the effector caspases (caspases-3, -6, -7) leading to the disassembly of the cell, and the other route involving the caspase-8 dependent cleavage of the pro-death Bcl-2 family protein, Bid, and engaging the mitochondrial or intrinsic death pathway.

In light of this cell death activity, molecules that bind to TRAIL-R1 and TRAIL-R2 may have a therapeutic role in the treatment of a wide variety of cancers. Accordingly, the CTLD polypeptide libraries of the invention were screen in an effort to identify and isolate CTLD-based polypeptides having specific binding activity to TRAILR1 and TRAIL R2.

### Example 9

### Panning & Screening of Human Library 1-4

Phage generated from human library 1-4 were panned on recombinant TRAIL R1 (DR 4)/Fc chimera, and TRAIL R2 (DR 5)/Fc chimera. Screening of these binding panels after three, four, and/or five rounds of panning using an ELISA plate assay identified receptor-specific binders in all cases.

### Example 10

### Construction of Libraries and Clones for Selection and Screening of Agonists for TRAIL Receptors DR 4 and DR 5

Phage libraries expressing linear or cyclized randomized peptides of varying lengths can be purchased commercially from manufacturers such as New England Biolabs (NEB). Alternatively, phage display libraries containing randomized peptides in loops of the C-type lectin domain (CTLD) of human tetranectin can be generated. Loops 1, 2, 3, and 4 of the LSA of CTLD are shown in FIG. 4. Amino acids within these loops can be randomized using an NNS or NNK overlapping PCR mutagenesis strategy. From one to seven codons in any one loop may be replaced by a mutagenic NNS or NNK codon to generate libraries for screening; alternatively, the number of mutagenized amino acids may exceed the number being replaced (two amino acids may be replaced by five, for example, to make larger randomized loops). In addition, more than one loop may be altered at the same time. The overlap PCR strategy can generate either a Kpn I site in the final DNA construct between loops 2 and 3, which alters one of the amino acids between the loops, exchanging a threonine for the original alanine. Alternatively, a BssH II site can be incorporated between loops 2 and 3 that does not alter the original amino acid sequence.

### Example 11

### Selection and Screening of Agonists for TRAIL Receptors DR 4 and DR 5

Bacterial colonies expressing phage were generated by infection or transfection of bacteria such as E. coli TG-1 or XL-1 Blue using either glycerol phage stocks of phage libraries or library DNA, respectively. Fifty milliliters of infected/transfected bacteria at an O.D.₆₀₀ of 1.0 are grown for 15 min at room temperature (RT), after which time 40% of the final concentration of selectable drug marker is added to the culture and incubated for 1 h at 37° C. Following that incubation the remaining drug for selection is added and incubated for another hour at 37° C. Helper phage VCS M13 are added and incubated for 2 h. Kanamycin (70 μg/mL) is added to the culture, which is then incubated overnight at 37° C. with shaking Phage are harvested by centrifugation followed by cold precipitation of phage from supernatant with one third volume of 20% polyethylene glycol (PEG) 8000/2.5 M NaCl. Phage are resuspended in a buffer containing a protease inhibitor cocktail (Roche Complete Mini EDTA-free) and are subsequently sterile filtered. Phage libraries are titered in E. coli TG-1, XL1-Blue, or other appropriate bacterial host.

Phage are panned in rounds of positive selection against human DR 4 and/or DR 5. Human DR 4 and DR 5 (aka human TRAIL death receptors 1 and 2) are commercially available in a soluble form (Antigenix America, Cell Sciences, or as Fc (Genway Biotech, R&D Systems) or GST fusions (Novus Biologicals). Soluble DR 4 or DR 5 in PBS is bound directly to a solid support, such as the bottom of a microplate well (Immulon 2B plates) or to magnetic beads such as Dynabeads. About 250 ng to 500 ng of soluble DR 4 or DR 5 is bound to the solid substrate by incubation overnight in PBS at either 4° C. or RT. The plates (or beads) are then washed three times in PBS/0.05% Tween 20, followed by addition of a blocking agent such as 1% BSA, 0.05% sodium azide in PBS and is incubated for at least 0.5 h at RT to prevent binding of material in future steps to non-specific surfaces. Blocking agents such as PBS with 3% non-fat dry milk or boiled casein can also be used.

In an alternative protocol, in order to bind DR 4 or DR 5 Fc fusion proteins, plates or beads are first incubated with 0.5-1 μg of a commercially available anti-Fc antibody in PBS. The plates (or beads) are washed and blocked with 1% BSA, 0.05% sodium azide in PBS as above, and are then incubated with death receptor fusion protein at 5 μg/mL and incubated for 2 h at RT. Plates are then washed three times with PBS/0.05% Tween 20.

Phage libraries at a concentration of about 10¹¹ or 10¹² pfu/mL are added to the wells (or beads) containing directly or indirectly bound death receptor. Phage are incubated for at least 2 h at RT, although to screen for different binding properties the incubation time and temperature can be varied. Wells are washed at least eight times with PBS/0.05% Tween 20, followed by PBS washes (8×). Wells can be washed in later rounds of selection with increasingly acidic buffers, such as 100 mM Tris pH 5.0, Tris pH 4.0, and Tris pH 3.0. Bound phages are eluted by trypsin digestion (100 μL of 1 mg/mL trypsin in PBS for 30 min). Bound phages can also be eluted using 0.1 M glycine, pH 2.2. Alternatively, bound phages can be eluted using TRAIL (available commercially from AbD Serotec) to select for CTLDs or peptides that compete with TRAIL for binding to the death receptors. Further, bound phage can be eluted with compounds that are known to compete with TRAIL for death receptor binding.

Eluted phage are incubated for 15 min with 10 mL of freshly grown bacteria at an OD₆₀₀ of 0.8, and the infected bacteria are treated as above to generate phage for the second round of panning Two or three additional rounds of positive panning are performed.

As an alternative to using DR 4 and/or DR 5 directly or indirectly bound to a support, DR 4 and/or DR 5 expressed endogenously by cancer cell lines or expressed by transfected cells such as 293 cells may be used in rounds of positive selection. For transfected cells, transfection is performed two days prior to panning using the Qiagen Attractene™ protocol, for example, and an appropriate expression plasmid such as pcDNA3.1, pCEP4, or pCEP5 bearing DR 4 or DR 5. Cells are dissociated in a non-trypsin dissociation buffer and 6×10⁶ cells are resuspended in 2 mL IMDM buffer. Phage to be panned are dialyzed prior to being added to cells and incubated for 2 h, RT. Cells are washed by pelleting and resuspending multiple times in IMDM, and phage are eluted with glycine buffer.

In order to select those peptides that have affinity for DR 4 and/or DR 5 but not decoy receptors, negative selection rounds or negative selection concomitant with positive selection are performed. Negative selection is done using the decoy receptors DcR1, DcR2, soluble DcR3, and/or osteoprotegerin (OPG, R&D systems). OPG and soluble DcR3 are commercially available (GeneTex, R&D systems), as are DcR1 and DcR2 conjugated to Fcor GST (R&D Systems, Novus Biologicals). For negative selection rounds, decoy receptor is bound to plates or beads and blocked as described above for positive rounds of selection. Beads are more desirable as a larger surface area of negative selection molecules can be exposed to the library being panned. The primary library or the phage from other rounds of positive selection are incubated with the decoy receptors for 2 h at room temperature, or overnight at 4° C. Unbound phage are then removed and subjected to a positive round of selection.

Positive selection is also performed simultaneously with negative selection. Wells or beads coated with soluble DR 4 or DR 5 are blocked and exposed to the primary library or phage from a selection round as described above, but a decoy receptor such as DcR1 is included at a concentration of 10 μg/mL. Incubation time may be extended from 2 h to several days at 4° C. prior to elution in this strategy in order to obtain phage with greater specificity and affinity for DR 4 or DR 5. Negative selection using DR 4, in order to obtain DR 5-specific, or DR 5, in order to obtain DR 4-specific binders, can also be performed using the approaches detailed above.

Negative selection can also be performed on cancerous or transfected cells that express one or more of the decoy receptors. Negative selection is performed similarly to positive selection as described above except that phage are recovered from the supernatant after spinning cells down after incubation and then used in a positive round of selection.

### Example 12

### Plasmid Construction of Trimeric TRAIL Receptor Agonists and Trimeric CTLD-Derived TRAIL Receptor Agonists

The various versions of trimeric TRAIL receptor agonists and trimeric CTLD-derived TRAIL receptor agonists from phage display or from peptide-grafted, peptide-trimerization domain (TD) fusions, peptide-TD-CTLD fusion, or their various combinations are sub-cloned into bacterial expression vectors (pT7 in house vector, or pET, NovaGen) and mammalian expression vectors (pCEP4, pcDNA3, Invitrogen) for small scale or large-scale production.

Primers are designed to PCR amplify DNA fragments of binders/agonists from various functional display vectors from Example 1. Primers for the 5′-end are flanked with BamH I restriction sites and are in frame with the leader sequence in the vector pT7CIIH6. 5′ primers also can be incorporated with a cleavage site for protease Granzyme B or Factor Xa. 3′ primers are flanked with EcoRI restriction sites. PCR products are digested with BamHI/EcoRI, and then ligated into pT7CIIH6 digested with the same enzymes, to create bacterial expression vectors pT7CIIH₆-TRAILa.

The TRAIL receptor agonist DNAs can be sub-cloned into vector pT7CIIH₆ or pET28a (NovoGen), without any leader sequences and 6×His. 5′ primers are flanked with NdeI restriction sites and 3′ primers are flanked with EcoRI restriction sites. PCR products are digested with NdeI/EcoRI, and ligated into the vectors digested with the same enzymes, to create expression vectors pT7-TRAILa and pET-TRAILa.

The TRAIL receptor agonist DNAs can be sub-cloned into vector pT7CIIH₆ or pET28a (NovoGen), with a secretion signal peptide. Expressed proteins are exported into bacterial periplasm, and secretion signal peptide is removed during translocation. 5′ primers are flanked with NdeI restriction sites and the primers are incorporated into a bacterial secretion signal peptide, PelB, OmpA or OmpT. 3′ primers are flanked with EcoRI restriction sites. A 6×His tag coding sequence can optionally be incorporated into the 3′ primers. PCR products are digested with NdeI/EcoRI, and ligated into vectors that are digested with the same enzymes, to create the expression vectors pT7-sTRAILa, pET-sTRAILa, pT7-sTRAILaHis, and pET-sTRAILHis.

The TRAIL receptor agonist DNAs can also be sub-cloned into mammalian expression vector pCEP4 or pcDNA3.1, along with a secretion signal peptide. Expressed proteins are secreted into the culture medium, and the secretion signal peptide is removed during the secretion processes. 5′ primers are flanked with NheI restriction sites and the primers are incorporated into a tetranectin secretion signal peptide, or another secretion signal peptide (e.g., Ig peptide). 3′ primers are flanked with XhoI restriction sites. A 6×His tag is optionally incorporated into the 3′ primers. PCR products are digested with NheI/XhoI, and ligated into the vectors that are digested with the same enzymes, to create expression vectors pCEP4-TRAILa, pcDNA-TRAILa, pCEP4-TRAILaHis, and pcDNA-TRAILaHis.

### Example 13

### Expression and Purification of TRAIL Receptor Agonists from Bacteria

Bacterial expression constructs are transformed into bacterial strain BL21(DE3) (Invitrogen). A single colony on a fresh plate is inoculated into 100 mL of 2×YT medium in a shaker flask. The flask is incubated in a shaker rotating at 250 rpm at 37° C. for 12 h or overnight. Overnight culture (50 mL) is used to inoculate 1 L of 2×YT in a 4 L shaker flask. Bacteria are cultured in the flask to an OD₆₀₀ of about 0.7, at which time IPTG is added to the culture to a final concentration of 1 mM. After a 4 h induction, bacterial pellets are collected by centrifugation and saved for subsequent protein purification.

Bacterial fermentation is performed under fed-batch conditions in a 10-liter fermentor. One liter of complex fermentation medium contains 5 g of yeast extract, 20 g of tryptone, 0.5 g of NaCl, 4.25 g of KH₂PO₄, 4.25 g of K₂HPO₄.3H₂O, 8 g of glucose, 2 g of MgSO₄.7H₂O, and 3 mL of trace metal solution (2.7% FeCl₃.6H₂O/0.2% ZnCl₂.4H₂O/0.2% CoCl₂.6H₂O/0.15% Na₂MoO₄.2H₂O/0.1% CaCl₂.2H₂O/0.1% CuCl₂/0.05% H₃BO₃/3.7% HCl). The fermentor is inoculated with an overnight culture (5% vol/vol) and grown at constant operating conditions at pH 6.9 (controlled with ammonium hydroxide and phosphoric acid) and at 30° C. The airflow rate and agitation are varied to maintain a minimum dissolved oxygen level of 40%. The feed (with 40% glucose) is initiated once the glucose level in the culture is below 1 g/L, and the glucose level is maintained at 0.5 g/L for the rest of the fermentation. When the OD₆₀₀ reaches about 60, IPTG is added into the culture to a final concentration of 0.05 mM. Four hours after induction, the cells are harvested. The bacterial pellet is obtained by centrifugation and stored at −80° C. for subsequent protein purification.

Expressed proteins that are soluble, secreted into the periplasm of the bacterial cell, and include an affinity tag (e.g., 6×His tagged proteins) are purified using standard chromatographic methods, such as metal chelation chromatography (e.g., Ni affinity column), anionic/cationic affinity chromatography, size exclusion chromatography, or any combination thereof, which are well known to one skilled in the art.

Expressed proteins can form insoluble inclusion bodies in bacterial cells. These proteins are purified under denaturing conditions in initial purification steps and undergo a subsequent refolding procedure, which can be performed on a purification chromatography column. The bacterial pellets are suspended in a lysis buffer (0.5 M NaCl, 10 mM Tris-HCl, pH 8, and 1 mM EDTA) and sonicated. The inclusion body is recovered by centrifugation, and subsequently dissolved in a binding buffer containing 6M guanidinium chloride, 50 mM Tri-HCl, pH8, and 0.1M DTT. The solubilized portion is applied to a Ni affinitycolumn. After washing the unbound materials from the column, the proteins are eluted with an elution buffer (6M guanidinium chloride, 50 mM Tris-HCl pH8.0, 10 mM 2-mercaptoethanol, 250 mM imidazole). Isolated proteins are buffer exchanged into the binding buffer, and are re-applied to the Ni⁺ column to remove the denaturing agent. Once loaded onto the column, the proteins are refolded by a linear gradient (0-0.5M NaCl) using 5 C.V. (column volumes) of a buffer that lacks the denaturant (50 mM Tris-HCl pH8.0, 10 mM 2-mercaptoethanol, plus 2 mM CaCl₂). The proteins are eluted with a buffer containing 0.5M NaCl, 50 mM Tris-HCl pH8.0, and 250 mM imidazole. The fusion tags (6×His, CII6His) are cleaved with Factor Xa or Granzyme B, and removed from protein samples by passage through a Ni⁺-NTA affinity column. The proteins are further purified by ion-exchange chromatography on Q-sepharose (GE) using linear gradients (0-0.5M NaCl) over 10 C.V. in a buffer (50 mM Tris-HCl, pH8.0 and 2 mM CaCl₂). Proteins are dialyzed into 1×PBS buffer. Optionally, endotoxin is removed by passing through a Mustang E filter (PALL).

To prepare soluble extracts from bacterial cells for expressed proteins in the periplasm, the bacterial pellets are suspended in a loading buffer (10 mM phosphate buffer pH6.0), and lysed using sonication (or alternatively a French press). After spinning down the insoluble portion in a centrifuge, the soluble extract is applied to an SP FF column (GE). Periplasmic extracts are also prepared by osmotic shock or “soft” sonication. Secreted soluble 6×His tagged proteins are purified by Ni⁺-NTA column as described above. Crude extracts are buffer exchanged into an affinity column loading buffer, and then applied to an SP FF column. After washing with 4 C.V. of loading buffer, the proteins are eluted using a 100% gradient over 8 C.V. with a high salt buffer (10 mM phosphate buffer, 0.5M NaCl, pH6.0). Eluate is filtered by passing through a Mustang E filter to remove endotoxin. The partially purified proteins are buffer exchanged into 10 mM phosphate buffer, pH7.4, and then loaded to a Q FF column. After washing with 7 C.V. with 10 mM phosphate buffer pH 6.0, the proteins are eluted using a 100% gradient over 8 C.V. with a high salt buffer (10 mM phosphate buffer, pH6.0, 0.5M NaCl). Once again endotoxin is removed by passing through a Mustang E filter.

### Example 14

### Expression and Purification of TRAIL Receptor Agonists from Mammalian Cells

Plasmids for each expression construct are prepared using a Qiagen Endofree Maxi Prep Kit. Plasmids are used to transiently transfect HEK293-EBNA cells. Tissue culture supernatants are collected for protein purification 2-4 days after transfection.

For large-scale production, stable cell lines in CHO or PER.C6 cells are developed to overexpress TRAIL receptor agonists. Cells (5×10⁸) are inoculated into 2.5 L of media in a 20 L bioreactor (Wave). Once the cells have doubled, fresh media (1× start volume) is added, and continues to be added as cells double until the final volume reaches 10 L. The cells are cultured for about 10 days until cell viability drops to 20%. The cell culture supernatant is then collected for purification.

Both His-tagged protein purification (by Ni⁺-NTA column) and non-tagged protein purification (by ion exchange chromatography) are employed as detailed above.

### Example 15

### Affinity Maturation of TRAIL Receptor Agonists Assisted by in Silico Modeling

In silico modeling is used to affinity mature TRAIL receptor agonists that are identified from the CTLD phage display library screening. Agonist homology models are built based on the known tetranectin 3D structures. Loop conformations of homology models of agonists are refined and optimized using LOOPER (DS2.1, Accelrys) and their related algorithms. This process includes three basic steps: 1. Construction of a set of possible loop conformers with optimized interactions of loop backbone with the rest of the protein; 2. Building and structural optimization of loop side chains and energy minimization applied to all loop atoms; 3. Final scoring and ranking the retained variants of loop conformers. Potential binding regions or epitopes located on the DR 4/DR 5 extracellular domain are identified for the agonists using a combination of manual and molecular dynamics-based docking. The binding domains are further confirmed by performing binding assays using deletion or point mutations of DR 4/DR 5 extracellular domain(s) and the agonists. Amino acid residues (or sequences) that are involved in determining binding specificity are defined on both DR 4/DR 5 and TRAIL CTLD agonists. A combination of random mutations at various target positions is screened using structure-based computation to determine the compatibility with the structure template. Based on the analysis of apparent packing defects, residues are selected for mutagenesis to construct a library for phage display.

The 3D models of TRAIL receptor agonist peptides and DR 4/DR 5 can be used as a reference to refine the peptide-grafted CTLD and DR 4/DR 5 modeling. When TRAIL receptor agonist peptides are grafted into CTLD loops, loop conformations are optimized and re-surfaced to match agonist peptides/DR 4/DR 5 binding by changing the flanking and surrounding amino acid residues using in silico modeling. Peptide grafted CTLD agonist homology models are built based on the known tetranectin 3D structures. Loop conformations of homology models of agonists are refined and optimized using LOOPER (DS2.1, Accelrys) and their related algorithms as described above. A combination of random mutations at various target positions is screened by structure-based computation for their compatibility with the structure template. Based on analysis of apparent packing defects, amino acid residues flanking and surrounding peptides are selected for mutagenesis to construct a library for phage display.

### Example 16

### Inhibition of Cancer Cell Proliferation

Human cancer cell lines expressing DR 4 and/or DR 5 such as COLO205 (colorectal adenocarcinoma), NCI-H2122 (non-small cell lung cancer), MIA PaCa-2 (pancreatic carcinoma), ACHN (renal cell carcinoma), WM793B (melanoma) and U266B1 (lymphoma) (all purchased from American Type Tissue Collection (Manassas, Va.) are cultured under the appropriate condition for each cell line and seeded at cell densities of 5,000-20,000 cells/well (as determined appropriate by growth curve for each cancer cell line). DR 4/5 agonistic molecules are added at concentrations ranging from 0.0001-100 μg/mL. Optionally DR 4/DR 5 agonists are combined with therapeutic methods, including chemotherapeutics (e.g., bortezomib) or cells that are pre-sensitized by radiation, to generate a synergistic effect that upregulates DR 4 or DR 5 or alters caspase activity. The number of viable cells is assessed after 24 and 48 h using “CellTiter 96® AQᵤₑₒᵤₛ One Solution Cell Proliferation Assay” (Promega) according to the manufacturer's instructions, and the IC₅₀ concentrations for the DR 4/DR 5 agonists are determined.

### Example 17

### Activation of Caspases by DR 5 and DR 4 Agonistic Molecules in Cancer Cell Lines

Human cancer cell lines expressing DR 4 and/or DR 5 such as COLO205 (colorectal adenocarcinoma), NCI-H2122 (non-small cell lung cancer), MIA PaCa-2 (pancreatic carcinoma), ACHN (renal cell carcinoma), WM793B (melanoma) and U266B1 (lymphoma) (all purchased from American Type Tissue Collection (Mannasas, Va.)) are cultured under the appropriate condition for each cell line and seeded at cell densities of 5,000-20,000 cells/well (as determined appropriate by growth curve for each cancer cell line). DR 4/5 agonistic molecules are added at concentrations ranging from 0.0001-100 μg/mL. DR 4/DR 5 agonists can be combined with other therapies such as chemotherapeutics (e.g., bortezomib) or cells that are pre-sensitized by radiation to determine whether such a combination has a synergistic effect on up-regulation of DR 4 or DR 5 or altering caspase activity. Caspase activity is determined at various timepoints using the “APO-ONE Caspase assay” (Promega) according to the manufacturers instruction.

Further analysis by Western Blot is performed by incubating 2×10⁶ tumor cells as described above. Subsequent cell lysates are prepared for Western Blot. Proteins are separated by SDS-PAGE and transferred to nitrocellulose membranes. The filters are incubated with antibodies that recognize the pro and cleaved forms of the apoptotic proteins PARP, caspase 3, caspase 8, caspase 9, bid and actin. The bands corresponding to specific proteins are detected by HRP-conjugated secondary antibodies and enhanced chemiluminescence.

### Example 18

### Agonist Molecule Assessment in Tumor Xenograft Models

Cancer cell lines (e.g. HCT-116, SW620, COLO205) are injected s.c into Balb/c nude or SCID mice. Tumor length and width is measured twice a week using a caliper. Once the tumor reaches 250 mm³ in size, mice will be randomized and treated i.v. or s.c. with 10-100 mg/kg DR 4 or DR 5 agonist. Treatment can be combined with other therapeutics such as chemotherapeutics (e.g. irinotecan, bortezomib, or 5FU) or radiation treatment. Tumor size is observed for 30 days unless tumor size reaches 1500 mm³ in which case mice have to be sacrificed.

### Example 19

### Panning of Human Library 1-4 on Human DR 4 and DR 5

1. Panning on DR 4 receptor

Panning was performed using the human Loop1-4 library of human CTLDs on DR 4/Fc antigen-coated (R&D Systems) wells prepared fresh the night before bound with 250 ng to 1 μg of the carrier free target antigen diluted in 100 μL of PBS per well. Antigen plates were incubated overnight at 4° C. then for 1 hour at 37° C., washed twice with PBS/0.05% Tween 20 and twice with PBS, and then blocked with 1% BSA/PBS for 1 hr at 37° C. prior to panning Six wells were used in each round, and phage were bound to wells for two hours at 37° C. using undiluted, 1:10, and 1:100 dilutions in duplicates of the purified phage supernatant stock. Since target antigens were expressed as Fc fusion proteins, phage supernatant stocks contained 1 μg/mL soluble IgG1 Fc acting as soluble competitor. In addition, prior to target antigen binding, phage supernatants were pre-bound to antigen wells with human IgG1 Fc to remove Fc binders (no soluble IgG1 Fc competitor was present during the pre-binding).

To produce phage for the initial round of panning, 10 μg of library DNA was transformed into electrocompetent TG-1 bacteria and grown in a 100 mL culture containing SB with 40 μg/mL carbenicillin and 2% glucose for 1 hour at 37° C. The carbenicillin concentration was then increased to 50 μg/mL and the culture was grown for an additional hour. The culture volume was then increased to 500 mL, and the culture was infected with helper phage at a multiplicity of infection (MOI) of 5×10⁹ pfu/mL and grown for an additional hour at 37° C. The bacteria were spun down and resuspended in 500 mL SB containing 50 μg/mL carbenicillin and 100 μg/mL kanamycin and grown overnight at room temperature shaking at 250 rpm. The following day bacteria were spun out and the phage precipitated with a final concentration of 4% PEG/0.5 M NaCl on ice for 1 hr. Precipitated phage were then spun down at 10,500 rpm for 20 minutes at 4° C. Phage pellets were resuspended in 1% BSA/PBS containing the Roche EDTA free complete protease inhibitors. Resuspended phage were then spun in a microfuge for 10 minutes at 13,200 rpm and passed through a 0.2 μM filter to remove residual bacteria.

50 μL of the purified phage supernatant stock per well were pre bound to the IgG Fc coated wells for 1 hr at 37° C. and then transferred to the target antigen coated well at the appropriate dilution for 2 hrs at 37° C. as described above. Wells were then washed with PBS/0.05% Tween 20 for 5 minutes pipeting up and down (1 wash at round 1, 5 washes at round 2, and 10 washes at rounds 3 and 4). Target antigen bound phage were eluted with 60 μL per well acid elution buffer (glycine pH 2) and then neutralized with 2M Tris 3.6 μL/well. Eluted phage were then used to infect TG-1 bacteria (2 mL at OD₆₀₀ of 0.8-1.0) for 15 minutes at room temperature. The culture volume was brought up to 10 mL in SB with 40 μg/mL carbenicillin and 2% glucose and grown for 1 hour at 37° C. shaking at 250 rpm. The carbenicillin concentration was then increased to 50 μg/mL and the culture was grown for an additional hour. The culture volume was then increased to 100 mL, and the culture was infected with helper phage at an MOI of 5×10⁹ pfu/mL and grown for an additional hour at 37° C. The bacteria were spun down and resuspended in 100 mL SB containing 50 μg/mL carbenicillin and 100 μg/mL kanamycin and grown overnight at room temperature with shaking at 250 rpm. Subsequent rounds of panning were performed similarly adjusting for smaller culture volumes, and with increased washing in later rounds. Clones were panned on DR 4/Fc for four rounds and clones obtained from screening rounds three and four.

2. Phage ELISA

Panning was performed using the TG-1 strain of bacteria for at least four rounds. At each round of panning sample titers were taken and plated on LB plates containing 50 μg/mL carbenicillin and 2% glucose. To screen for specific binding of phagemid clones to the receptor target, individual colonies were picked from these titer plates from the later rounds of panning and grown up overnight at room temperature with shaking at 250 rpm in 250 μL of 2×YT medium containing 2% glucose and 50 μg/mL carbenicillin in a polypropylene 96-well plate with an air-permeable membrane on top. The following day a replica plate was set up in a 96-deep-well plate by inoculating 500 μL of 2×YT containing 2% glucose and 50 μg/mL carbenicillin with 30 μL of the previous overnight culture. The remaining overnight culture was used to make a master stock plate by adding 100 μL of 50% glycerol to each well and storing at −80° C. The replica culture plate was grown at 37° C. with shaking at 250 rpm for approximately 2 hrs until the OD₆₀₀ was 0.5-0.7. The wells were then infected with K07 helper phage to 5×10⁹ pfu/mL mixed and incubated at 37° C. for 30 minutes without shaking, then incubated an addition 30 minutes at 37° C. with shaking at 250 rpm. The cultures were then spun down at 2500 rpm and 4° C. for 20 minutes. The supernatants were removed from the wells and the bacterial cell pellets were re-suspended in 500 μL of 2×YT containing 50 μg/mL carbenicillin and 50 μg/mL kanamycin. An air-permeable membrane was placed on the culture block and cells were grown overnight at room temperature with shaking at 250 rpm.

On day 3, cultures were spun down and supernatants containing the phage were blocked with 3% milk/PBS for 1 hr at room temperature. An initial Phage ELISA was performed using 75-100 ng of antigen bound per well. Non-specific binding was measured using 75-100 ng of human IgG1 Fc per well. DR 4/Fc antigen (R&D Systems)-coated wells and IgG Fc coated wells were prepared fresh the night before by binding the above amount of antigen diluted in 100 μL of PBS per well. Antigen plates were incubated overnight at 4° C. then for 1 hour at 37° C., washed twice with PBS/0.05% Tween 20 and twice with PBS, and then blocked with 3% milk/PBS for 1 hr at 37° C. prior to the ELISA. Blocked phage were bound to blocked antigen-bound plates for 1 hr then washed twice with 0.05% Tween 20/PBS and then twice more with PBS. A HRP-conjugated anti-M13 secondary antibody diluted in 3% milk/PBS was then applied, with binding for 1 hr and washing as described above. The ELISA signal was developed using 90 μL TMB substrate mix and then stopped with 90 μL 0.2 M sulfuric acid, then ELISA plates were read at 450 nM. Secondary ELISA screens were performed on the positive binding clones identified, screening against additional TRAIL receptors and decoy receptors to test for specificity (DR 4, DR 5, DcR1 and DcR2). Secondary ELISA screens were performed similarly to the protocol detailed above.

DR 4 specific binding clones. Examples of amino acid sequences for Loops 1 and 4 selected for specific binding to the DR 4 receptor from the human TN1-4 library are detailed below in Table 5.

TABLE 5
Sequences of Loops 1 and 4 from binders
to human DR4

| | | Loop 1 | | Loop 4 |
|---|---|---|---|---|
| | Loop 1 | SEQ ID | Loop 4 | SEQ ID |
| Clones | Sequence | NO | Sequence | NO |
| 014-42.3D11 | GWLEGAGW | 218 | DGGWHWRWEN | 219 |
| 014-42.3B8 | GWLEGVGW | 220 | DGGEHWGWEN | 221 |
| 014-42.3D9 | GYLAGVGW | 222 | DGGRGFRWEN | 223 |
| 014-42.3C7 | GWLEGYGW | 224 | DGGTWWEWEN | 225 |
| 014-42.3D10 | GYLEGYGW | 226 | DGGATIAWEN | 227 |
| 014-42.3G8 | GWLqGVGW | 228 | DGGRGWPWEN | 229 |
| 014-40.3E11 | GYLAGYGW | 230 | DGGPSIWREN | 231 |
| 014-40.3B2 | GYIEGTGW | 232 | DGGSNWAWEN | 233 |
| 014-40.3B3 | GYMSGYGW | 234 | DGGMMARWEN | 235 |
| 014-40.3A3 | GFMVGRGW | 236 | DGGSMWPWEN | 237 |
| 014-40.3H2 | MVTRPPYW | 238 | DGGWVMSFEN | 239 |
| 014-40.3E9 | PFRVPqWW | 240 | DGGYGPVqEN | 241 |
| 064-40.2G11 | GWLEGAGW | 218 | DGGWQWRWEN | 242 |
| 064-40.2E10 | GYLDGVGW | 243 | DGGQGCRWEN | 244 |
| 064-36.1E4 | VLRLAWSW | 245 | DGGKRNGCEN | 246 |
| 064-40.1E11 | WLSLFSPW | 247 | DGGRGVRGEN | 248 |
| 064-36.1B7 | GWMAGVGW | 249 | DGGRRLPWEN | 250 |
| 064-40.2C7 | SYRLHYGW | 251 | DGGRRWLGEN | 252 |
| 064-36.1E1 | IWPLRFRW | 253 | DGGFVTRKEN | 254 |
| 064-40.2D9 | WqLYYRYW | 255 | DGGVGCMVEN | 256 |
| 064-36.1G4 | RCLqGVGW | 257 | DGGRGWPWEN | 229 |
| 064-36.1E12 | GCTqGQGW | 258 | DGGKKWKWEN | 259 |
| 064-21.1A5 | GFLqGNGW | 260 | DGGMWDRWEN | 261 |
| 064-40.2A10 | GVLqRGGW | 262 | DGGPGGEREN | 263 |
| 064-40.2C3 | PFRVLqQWW | 264 | DGGCGPVqQEN | 265 |
| 064-40.2D2 | PFRGPqQWW | 266 | DGGYGPVGEN | 267 |
| 064-40.2E5 | ARFAMWqQW | 268 | DGGRAGVGEN | 269 |
| 064-40.2C4 | GWLQGYGW | 270 | DGGqQIGWGEN | 271 |
| 064-40.2C5 | AWRSWLNW | 272 | DGGREqQRREN | 273 |
| 029-61.1E11 | GWLEGVGW | 220 | DGGWPFSNEN | 274 |
| 029-61.1A5 | GWLMGTGW | 275 | DGGWWNRWEN | 276 |
| 029-62.2C5 | VRRMGFHW | 277 | DGGRVAVGEN | 278 |
| 029-62.2B3 | RYHVQALW | 279 | DGGRVRPREN | 280 |
| 029-62.4F5 | IqCSPPLW | 281 | DGGAVqqQEN | 282 |
| 029-62.7D10 | GLARQqGW | 283 | DGGKGRPREN | 284 |
| 064-40.1G9 | GWLSGVGW | 285 | DGGWAHAWEN | 286 |
| 064-40.1C7 | GWLEGVGW | 220 | DGGGGVRWEN | 287 |
| 064-98.1G6 | GWLSGYGW | 288 | DGGRVWSWEN | 289 |
| 064-99.2H5 | GLLSDWWW | 290 | DGGGNqSREN | 291 |
| 064-101.4B10 | QWVAFWSW | 292 | DGGSAVSGEN | 293 |
| 064-101.4H1 | PYTSWGLW | 294 | DGGVGGRGEN | 295 |
| 064-40.1G11 | VARWLLKW | 296 | DGGMCKPCEN | 297 |
| 064-36.1E10 | GFLAGVGW | 298 | DGGWWTRWEN | 299 |
| 064-36.1G10 | GYLQGSGW | 300 | DGGWKTRWEN | 301 |
| 064-36.1D7 | VRHWLqLW | 302 | DGGGWWKGEN | 303 |

3. Panning on DR 5 Receptor

Panning on the DR 5 receptors was performed similarly to that detailed above for the DR 4 receptor with the exception that five rounds of panning were performed and pre-binding was performed on wells coated with BSA rather than IgG1 Fc. However phage supernatant stocks contained soluble IgG1 Fc to act as soluble competitor for Fc binding during each round. DR 5-specific binding clones were obtained screening from round 5. Amino acid sequences for Loops 1 and 4 obtained from the clones for DR 5 specific binding are shown below in Table 6, below.

TABLE 6
Sequences of Loops 1 and 4 from binders
to human DR5

| | | Loop 1 | | Loop 4 |
|---|---|---|---|---|
| | Loop 1 | SEQ ID | Loop 4 | SEQ ID |
| Clone | Sequence | NO | Sequence | NO |
| 029-15.A3C | RATLRPRW | 304 | DGG----KN | 305 |
| 029-15.A7D | RAMLRSRW | 306 | DGGRWFQGKN | 307 |
| 029-15.A5A | RALFRPRW | 308 | DGGPWYLKEN | 309 |
| 029-15.A1H | RAVLRPRW | 310 | DGGWVLGGKN | 311 |
| 029-15.A8G | RAWLRPRW | 312 | DGGTLVSGEN | 313 |
| 029-15.B10A | RVIRRSMW | 314 | DGGQKWMAEN | 315 |
| 029-15.B2H | RVLQRPVW | 316 | DGGMVWSMEN | 317 |
| 029-15.B12H | RVqLRPRW | 318 | EGGFRRHAKN | 319 |
| 029-15.A6C | RVVRLSEW | 320 | DGGMLWAMEN | 321 |
| 029-15.B3G | RVISAPVW | 322 | DGGQQWAMEN | 323 |
| 029-15.B12G | RVLRRPQW | 324 | NGGDWRIPEN | 325 |
| 029-15.A6B | RVMMRPRW | 326 | DGGMWGAMEN | 327 |
| 029-15.B4F | RVMRRVLW | 328 | DGGRRETMKN | 329 |
| 029-15.A9G | RVMRRPLW | 330 | DGGRGQQWEN | 331 |
| 029-15.B11F | RVMRRREW | 332 | DGAQLMALEN | 333 |
| 029-15.B11C | RVWRRSLW | 334 | DGGHLVKQKN | 335 |
| 029-15.A4G | KRRWYGGW | 336 | DGGVNTVREN | 337 |
| 029-15.B9F | KRVWYRGW | 338 | DGGMRRRREN | 339 |
| 029-15.A9B | AVIRRPLW | 340 | DGGMKYTMEN | 341 |
| 029-15.B4H | ELVTSRLW | 342 | DGGVMqLGEN | 343 |
| 029-15.B11G | ELGTSRLW | 344 | DGGVMqLGEN | 343 |
| 029-15.B3A | FRGWLRWW | 345 | DDGARVLAEN | 346 |
| 029-15.B1A | GRLKGIGW | 347 | DGGRPQWGEN | 348 |
| 029-15.A4E | GVWqSFPW | 349 | DGGLGYLREN | 350 |
| 029-15.B3E | HLVSLAPW | 351 | DGGGMHQGKN | 352 |
| 029-15.A11H | HIFIDWGW | 353 | DGGVMTMGEN | 354 |
| 029-15.B4D | PVMRGVTW | 355 | DGGRSWVWEN | 356 |
| 029-15.A2E | QLVTVGPW | 357 | DGGVMHRTEN | 358 |
| 029-15.A7F | QLVVqMGW | 359 | DGGWMTVGEN | 360 |
| 029-15.B11A | VAIRRSVW | 361 | DGGERAHSEN | 362 |
| 029-15.B2B | WVMRRPLW | 363 | DGGSMGWREN | 364 |
| 029-15.A8E | WRSMVVWW | 365 | DGGKHTLGEN | 366 |
| 029-15.B3D | ELRTDGLW | 367 | DGGVMRRSEN | 368 |

As stated above, Loop 1 contained seven randomized amino acids in the screened library, whereas Loop 4 had an insertion of 5 randomized amino acids in place of 2 native amino acids (underlined regions in Table 6). In some clones having a glutamine (Q) in an altered loop, an amber-suppressible stop codon (TAG) encoded the glutamine, and this is indicated by a lower case “q”. During panning, a few clones containing changes outside of these regions were identified, for example, in Loop 4, the carboxy-flanking amino acid has been altered from E to K in several instances.

### Example 20

### Subcloning and Production of ATRIMER™ Binders to Human DR 4 and DR 5 Receptors

The loop region DNA fragments were released from DR 4/DR 5 binder DNA by double digestion with BglII and MfeI restriction enzymes, and were ligated to bacterial expression vectors pANA4 (SEQ ID: 54), pANA10 (SEQ ID NO: 60) or pANA19 to produce secreted ATRIMER™ in E. coli.

The expression constructs were transformed into E. coli strains BL21 (DE3), and the bacteria were plated on LB agar with ampicillin. Single colony on a fresh plate was inoculated into 2×YT medium with ampicillin. The cultures were incubated at 37° C. in a shaker at 200 rpm until OD600 reached 0.5, then cooled to room temperature. Arabinosis was added to a final concentration of 0.002-0.02%. The induction was performed overnight at room temperature with shaking at 120-150 rpm, after which the bacteria were collected by centrifugation. The periplasmic proteins were extracted by osmotic shock or gentle sonication.

The 6×His-tagged ATRIMERs™ were purified by Ni⁺-NTA affinity chromatography. Briefly, periplasmic proteins were reconstituted in a His-binding buffer (100 mM HEPES, pH 8.0, 500 mM NaCl, 10 mM imidazole) and loaded onto a Ni⁺-NTA column pre-equivalent with His-binding buffer. The column was washed with 10× vol. of binding buffer. The proteins were eluted with an elution buffer (100 mM HEPES, pH 8.0, 500 mM NaCl, 500 mM imidazole). The purified proteins were dialyzed into PBS buffer and bacterial endotoxin was removed by anion exchange.

The strep II-tagged ATRIMERs™ were purified by Strep-Tactin affinity chromatography. Briefly, periplasmic proteins were reconstituted in 1× binding buffer (20 mM Tris-HCl, pH 8.5, 150 mM NaCl, 2 mM CaCl₂, 0.1% Triton X-100) and loaded onto a Strep-Tactin column pre-equivalent with binding buffer. The column was washed with 10× vol. of binding buffer. The proteins were eluted with an elution buffer (binding buffer with 2.5 mM desthiobiotin). The purified proteins were dialyzed into binding buffer and bacterial endotoxin was removed by anion exchange.

The DNA fragments of loop region were sub-cloned into mammalian expression vectors pANA2 (SEQ ID NO: 52) and pANA11 (SEQ ID NO: 61) to produce ATRIMERs™ in a HEK293 transient expression system. The DNA fragments of the loop region were released from IL-23R binder DNA by double digestion with BglII and MfeI restriction enzymes, and ligated to the expression vectors pANA2 and pANA11, which were pre-digested with BglII and MfeI. The expression plasmids were purified from bacteria by Qiagen HiSpeed Plasmid Maxi Kit (Qiagene). For HEK293 adhesion cells, the transient transfection was performed by Qiagen SuperFect Reagent (Qiagene) according to the manufacturer's protocol. The day after transfection, the medium was removed and changed to 293 Isopro serum-free medium (Irvine Scientific). Two days later, 20% glucose in 0.5M HEPES was added into the media to a final concentration of 1%. The tissue culture supernatant was collected 4-7 days after transfection for purification. For HEK293F suspension cells, the transient transfection was performed by Invitrogen's 293Fectin and its protocol. The next day, 1× volume of fresh medium was added into the culture. The tissue culture supernatant was collected 4-7 days after transfection for purification. The His- or Strep II-tagged ATRIMER™ purification from mammalian tissue culture supernatant was performed as described above.

The DNA fragments of loop region were sub-cloned into mammalian expression vectors pANA5 (SEQ ID NO: 55), pANA6 (SEQ ID NO: 56), pANA7 (SEQ ID NO: 57), pANA8 (SEQ ID NO: 58) and pANA9 (SEQ ID NO: 59) to produce ATRIMER™ complexes with different CTLD-presenting orientations in the HEK293 transient expression system. pANA5 is a modified pCEP4 vector containing a C-terminal His-tag and a V₄₉ deletion in human TN. Similarly, pANA6 has a T₄₈ deletion, and pANA7 has T₄₈ and V₄₉ deletions. pANA8 has a C₅₀,C₆₀→S₅₀,S₆₀ double mutation to provide a more flexible CTLD than wildtype TN. pANA9 has E₁-V₁₇ deletions to remove the glycosylation site. The DNA fragments of loop region were released from IL-23R binder DNA by double digestion with BglII and MfeI restriction enzymes, and were ligated to the expression vectors pANA5, pANA6, pANA7, pANA8 and pANA9, which were pre-digested with BglII and MfeI.

### Example 21

### Characterization of the Affinity of Human DR 4 and DR 5 Receptor Binders Using Biacore

Apparent affinities of the trimeric DR 4 and DR 5 binders are provided in Tables 7 and 8, respectively. Immobilization of an anti-human IgG Fc antibody (Biacore) to the CM5 chip (Biacore) was performed using standard amine coupling chemistry and this surface was used to capture recombinant human DR 4 or DR 5 receptor Fc fusion protein (R&D Systems). ATRIMER™ COMPLEX dilutions (1-500 nM) were injected over the IL-23 receptor surface at 30 μl/min and kinetic constants were derived from the sensorgram data using the Biaevaluation software (version 3.1, Biacore). Data collection was 3 minutes for the association and 5 minutes for dissociation. The anti-human IgG surface was regenerated with a 30 s pulse of 3 M magnesium chloride. All sensorgrams were double-referenced against an activated and blocked flow-cell as well as buffer injections.

TABLE 7
Apparent affinities of DR4 receptor binders from H Loop 1-4 library.

| Analyte | Kₐ (1/M · s) | K_{d} (1/s) | K_{A} (1/M) | K_{D} (nM) |
|---|---|---|---|---|
| 014-42.3D10 | 1.22E+04 | 1.85E−03 | 6.58E+06 | 152 |
| 014-42.3B8 | 1.12E+05 | 1.01E−03 | 1.11E+08 | 9.01 |
| 014-42.3D11 | 1.33E+04 | 5.26E−04 | 2.53E+07 | 39.5 |

TABLE 8
Apparent affinities of DR5 receptor binders from H Loop 1-4 library.

| Analyte | Kₐ (1/M · s) | K_{d} (1/s) | K_{A} (1/M) | K_{D} (nM) |
|---|---|---|---|---|
| 1a7b (=A8G) | 4.05E+04 | 6.29E−04 | 6.43E+07 | 15.6 |
| 8b6b (=A1H) | 1.29E+04 | 5.06E−04 | 2.56E+07 | 39.1 |
| 9b3d (=B3D) | 116 | 1.04E−04 | 1.11E+06 | 899 |
| 2a1a (=B9F) | 4.38E+04 | 1.84E−03 | 2.38E+07 | 42.8 |
| 4a8c (=A3C) | 6.30E+04 | 3.62E−04 | 1.74E+08 | 5.74 |

Description of Cell Assay.

H2122 lung adenocarnoma cells (ATCC# CRL-5985) and A2780 ovarian carcinoma cells (European Collection of Cell Culture, #93112519) were incubated at 1×10⁴ cells/well with DR 5 ATRIMERs™ (20 μg/mL) or TRAIL (0.2 μg/mL, R&D Systems) in 10% FBS/RMPI media (Invitrogen) in a 96-well white opaque plate (Costar). The control wells received media and the respective buffer: TBS for DR 5 ATRIMERs™ and PBS for TRAIL. After 20 hours, cell viability was determined by ViaLight Plus (Lonza) and detected on a Glomax luminometer (Promega). Data were expressed as percent cell death relative to the respective buffer control. (See FIG. 10). The mean and standard error of triplicates were plotted using Excel. Five DR 5 ATRIMER™ COMPLEX were tested: 4a8c, 2a1a, 1a7b, 9b3d and 8b6b. Three DR 5 ATRIMERs™ (4a8c, 1a7b and 8b6b) showed over 50% killing in both cell lines. Similar data were obtained in a separate experiment.

### Example 22

### Panning of NEB Peptide Libraries on Human DR 5 and Identification of a DR 5 Specific Peptide

Panning of peptide libraries was performed using the New England Biolabs (NEB) Ph.D. Phage Display Libraries. Panning was performed on DR 5/Fc antigen-coated (R&D Systems) wells prepared fresh the night before bound with 3 μg of the carrier free target antigen diluted in 150 μL of 0.1M NaHCO₃ pH 8.6 per well. Duplicate wells were used in each round. Antigen plates were incubated overnight at 4° C. then for 1 hour at 37° C. The antigen was removed and the well was then blocked with 0.5% boiled Casein in PBS pH 7.4 for 1 hr at 37° C. prior to panning. The Casein was then removed and wells were then washed 6× with 300 μL of TBST (0.1% Tween), then phage were added. Since target antigens were expressed as Fc fusion proteins, prior to target antigen binding, phage supernatants were pre-bound for 1 hr to antigen wells with human IgG1 Fc to remove Fc binders (during rounds 2 through 4). Fc antigen bound wells were prepared similar to DR 5/Fc antigen bound wells as detailed above.

For the initial round of panning, 100 μL of TBST (0.1% Tween) was added to each well and 5 ul of each of the 3 NEB peptide libraries (Ph.D.-7, Ph.D.-12, and Ph.D.-C7C) were added to each well. The plate was rocked gently for 1 hr at room temperature, then washed 10× with TBST (0.1% Tween). Bound phage were eluted with 100 μL of PBS containing soluble DR 5/Fc target antigen at a concentration of 100 μg/ml. Phage were eluted for 1 hr rocking at room temperature. Eluted phage were then removed from the wells and used to infect 20 mls of ER2738 bacteria at an OD₆₀₀ₙₘ of 0.05 to 0.1, and grown shaking at 250 rpm at 37° C. for 4.5 hrs. Bacteria were then spun out of the culture at 12K×G for 20 min at 4° C. Bacteria were transferred to a fresh tube and re-spun. The supernatant was again transferred to a fresh tube and the Phage were precipitated by adding ⅙^{th} the volume of 20% PEG/2.5M NaCl. Phage were precipitated overnight at 4° C. The following day the precipitated phage were spun down at 12K×G for 20 min at 4° C. The supernatant was discarded and the phage pellet re-suspended in 1 ml of TBST (0.1% Tween). Residual bacteria were cleared by spinning in a microfuge at 13.2K for 10 minutes at 4° C. The phage supernatant was then transferred to a new tube and re-precipitated by adding ⅙th the volume of 20% PEG/2.5M NaCl, and incubating at 4° C. on ice for 1 hr. The precipitated phage were spun down in a microfuge at 13.2K for 10 minutes at 4° C. The supernatant was discarded and the phage pellet re-suspended in 200 μL of TBS. Subsequent rounds of panning were performed similar to round 1 with the exception phage were pre-bound for 1 hr to Fc coated wells and that 4 μL of the amplified phage stock from the previous round were used per well during the binding. In addition the tween concentration was increased to 0.5% in the TBST used during the 10 washes.

Phage ELISA

Panning was performed using the ER2738 strain of bacteria for at least four rounds. At each round of panning sample titers were taken and plated using top agar on LB/Xgal plates to obtain plaques. To screen for specific binding of phage clones to the receptor target, individual plaques were picked from these titer plates from the later rounds of panning and used to infect ER2738 bacteria at an OD₆₀₀ₙₘ of 0.05 to 0.1, and grown shaking at 250 rpm at 37° C. for 4.5 hrs. Then stored at 4° C. overnight.

On day 2, cultures were spun down at 12K×G for 20 min at 4° C., and supernatants containing the phage were blocked with 3% milk/PBS for 1 hr at room temperature. An initial Phage ELISA was performed using 75-100 ng of DR 5/Fc antigen bound per well. Non-specific binding was measured using wells containing 75-100 ng of human IgG1 Fc petr well. DR 5/Fc antigen (R&D Systems)-coated wells and IgG1 Fc coated wells were prepared fresh the night before by binding the above amount of antigen diluted in 100 μL of PBS per well. Antigen plates were incubated overnight at 4° C. then for 1 hour at 37° C., washed twice with PBS/0.05% Tween 20 and twice with PBS, and then blocked with 3% milk/PBS for 1 hr at 37° C. prior to the ELISA. Blocked phage were bound to blocked antigen-bound plates for 1 hr then washed twice with 0.05% Tween 20/PBS and then twice more with PBS. A HRP-conjugated anti-M13 secondary antibody diluted in 3% milk/PBS was then applied, with binding for 1 hr and washing as described above. The ELISA signal was developed using 90 μL TMB substrate mix and then stopped with 90 μL 0.2 M sulfuric acid, then ELISA plates were read at 450 nM. Secondary ELISA screens were performed on the positive binding clones identified, screening against additional TRAIL receptors and decoy receptors to test for specificity (DR 4, DR 5, DcR1 and DcR2). Secondary ELISA screens were performed similarly to the protocol detailed above.

DR 5 specific binding clone. An example of the amino acid sequence of a peptide from the NEB Ph.D.-C7C phage library selected for specific binding to the DR receptor is detailed below in Table 9.

TABLE 9

| | | Peptide |
|---|---|---|
| | Peptide | SEQ ID |
| Clone | Sequence | NO |
| 088-13.1H3 | ACFPIMTLHCGGG | 369 |

TABLE 10
TRAIL-Related Sequences

| Sequence | | SEQ ID |
|---|---|---|
| Description | Sequence | NO: |
| Human TRAIL | MAMMEVQGGP SLGQTCVLIV IFTVLLQSLC VAVTYVYFTN | 370 |
| GenBank Acc. | ELKQMQDKYS KSGIACFLKE DDSYWDPNDE ESMNSPCWQV | |
| P50591 | KWQLRQLVRK MILRTSEETI STVQEKQQNI SPLVRERGPQ | |
| 281 AA | RVAAHITGTR GRSNTLSSPN SKNEKALGRK INSWESSRSG | |
| | HSFLSNLHLR NGELVIHEKG FYYIYSQTYF RFQEEIKENT | |
| | KNDKQMVQYI YKYTSYPDPI LLMKSARNSC WSKDAEYGLY | |
| | SIYQGGIFEL KENDRIFVSV TNEHLIDMDH EASFFGAFLV G | |
| DR4; TRAIL-R1 | MAPPPARVHL GAFLAVTPNP GSAASGTEAA AATPSKVWGS | 371 |
| GenBank Acc. | SAGRIEPRGG GRGALPTSMG QHGPSARARA GRAPGPRPAR | |
| O00220 | EASPRLRVHK TFKFVVVGVL LQVVPSSAAT IKLHDQSIGT | |
| 468 AA | QQWEHSPLGE LCPPGSHRSE HPGACNRCTE GVGYTNASNN | |
| | LFACLPCTAC KSDEEERSPC TTTRNTACQC KPGTFRNDNS | |
| | AEMCRKCSRG CPRGMVKVKD CTPWSDIECV HKESGNGHNI | |
| | WVILVVTLVV PLLLVAVLIV CCCIGSGCGG DPKCMDRVCF | |
| | WRLGLLRGPG AEDNAHNEIL SNADSLSTFV SEQQMESQEP | |
| | ADLTGVTVQS PGEAQCLLGP AEAEGSQRRR LLVPANGADP | |
| | TETLMLFFDK FANIVPFDSW DQLMRQLDLT KNEIDVVRAG | |
| | TAGPGDALYA MLMKWVNKTG RNASIHTLLD ALERMEERHA | |
| | KEKIQDLLVD SGKFIYLEDG TGSAVSLE | |
| DRS; TRAIL-R2 | MEQRGQNAPA ASGARKRHGP GPREARGARP GPRVPKTLVL | 372 |
| GenBank Acc. | VVAAVLLLVS AESALITQQD LAPQQRAAPQ QKRSSPSEGL | |
| O14763 | CPPGHHISED GRDCISCKYG QDYSTHWNDL LFCLRCTRCD | |
| 440 AA | SGEVELSPCT TTRNTVCQCE EGTFREEDSP EMCRKCRTGC | |
| | PRGMVKVGDC TPWSDIECVH KESGTKHSGE APAVEETVTS | |
| | SPGTPASPCS LSGIIIGVTV AAVVLIVAVF VCKSLLWKKV | |
| | LPYLKGICSG GGGDPERVDR SSQRPGAEDN VLNEIVSILQ | |
| | PTQVPEQEME VQEPAEPTGV NMLSPGESEH LLEPAEAERS | |
| | QRRRLLVPAN EGDPTETLRQ CFDDFADLVP FDSWEPLMRK | |
| | LGLMDNEIKV AKAEAAGHRD TLYTMLIKWV NKTGRDASVH | |
| | TLLDALETLG ERLAKQKIED HLLSSGKFMY LEGNADSAMS | |
| TRAIL-R3 | MARIPKTLKF VVVIVAVLLP VLAYSATTAR QEEVPQQTVA | 373 |
| GenBank Acc. | PQQQRHSFKG EECPAGSHRS EHTGACNPCT EGVDYTNASN | |
| O14798 | NEPSCFPCTV CKSDQKHKSS CTMTRDTVCQ CKEGTFRNEN | |
| 259 AA | SPEMCRKCSR CPSGEVQVSN CTSWDDIQCV EEFGANATVE | |
| | TPAAEETMNT SPGTPAPAAE ETMNTSPGTP APAAEETMTT | |
| | SPGTPAPAAE ETMTTSPGTP APAAEETMTT SPGTPASSHY | |
| | LSCTIVGIIV LIVLLIVFV | |
| TRAIL-R4 | MGLWGQSVPT ASSARAGRYP GARTASGTRP WLLDPKILKF | 374 |
| GenBank Acc. | VVFIVAVLLP VRVDSATIPR QDEVPQQTVA PQQQRRSLKE | |
| Q9UBN6 | EECPAGSHRS EYTGACNPCT EGVDYTIASN NLPSCLLCTV | |
| 386 AA | CKSGQTNKSS CTTTRDTVCQ CEKGSFQDKN SPEMCRTCRT | |
| | GCPRGMVKVS NCTPRSDIKC KNESAASSTG KTPAAEETVT | |
| | TILGMLASPY HYLIIIVVLV IILAVVVVGF SCRKKFISYL | |
| | KGICSGGGGG PERVHRVLFR RRSCPSRVPG AEDNARNETL | |
| | SNRYLQPTQV SEQEIQGQEL AELTGVTVES PEEPQRLLEQ | |
| | AEAEGCQRRR LLVPVNDADS ADISTLLDAS ATLEEGHAKE | |
| | TIQDQLVGSE KLFYEEDEAG SATSCL | |
| OPG | MNNLLCCALV FLDISIKWTT QETFPPKYLH YDEETSHQLL | 375 |
| GenBank Acc. | CDKCPPGTYL KQHCTAKWKT VCAPCPDHYY TDSWHTSDEC | |
| NP_002537 | LYCSPVCKEL QYVKQECNRT HNRVCECKEG RYLEIEFCLK | |
| 401 AA | HRSCPPGFGV VQAGTPERNT VCKRCPDGFF SNETSSKAPC | |
| | RKHTNCSVFG LLLTQKGNAT HDNICSGNSE STQKCGIDVT | |
| | LCEEAFFRFA VPTKFTPNWL SVLVDNLPGT KVNAESVERI | |
| | KRQHSSQEQT FQLLKLWKHQ NKDQDIVKKI IQDIDLCENS | |
| | VQRHIGHANL TFEQLRSLME SLPGKKVGAE DIEKTIKACK | |
| | PSDQILKLLS LWRIKNGDQD TLKGLMHALK HSKTYHFPKT | |
| | VTQSLKKTIR FLHSFTMYKL YQKLFLEMIG NQVQSVKISC L | |

Examples 23-32 provide exemplary methods for identifying and isolating CTLD polypeptides that specifically bind IL-23 receptors using the combinatorial polypeptide libraries of the invention.

IL-23 is an essential cytokine for generation and survival of Th17 cells. There is mounting evidence from preclinical models and clinical experience that Th17 cells play a critical role in pathology of many autoimmune diseases, including rheumatoid arthritis, inflammatory bowel disease, psoriasis, systemic lupus erythematosus (SLE) and multiple sclerosis. IL-23R is a key target on Th17 cells. Similarly, the IL-23 cytokine is composed of two subunits: p19 and p40, with the p19 subunit being unique to IL-23, and p40 shared with IL-12. The IL-23 receptor is a heterodimeric receptor that binds IL-23 and mediates activation of certain T cell subsets, NK cells and myeloid cells. The IL-23 heterodimeric receptor is composed of two subunits: IL-23R and IL-12Rβ1, with IL-23R being the subunit unique to the IL-23 pathway. IL-12Rβ1 is shared with the IL-12 receptor and hence the IL-12 pathway.

Importantly, genetic variation in IL-23R has been associated with susceptibility to psoriasis and Crohn's disease and also has been implicated in susceptibility to ankylosing spondylitis, Vogt-Koyanagi-Harada disease, Systemic Sclerosis, Behçet's disease (BD), Primary Sjögren's Syndrome, Goodpasture disease. Also, importance of IL-23 in Graft Versus Host disease and chronic ulcers has been suggested, and IL-23 has been implicated in tumorigenesis.

Blockade of the IL-23 pathway is efficacious in many preclinical models of autoimmune disease. However, the nature of shared ligand and receptor subunits between IL-23 and IL-12 pathways has led to more complex biology than previously appreciated, and separation of IL-23 blockade from IL-12 blockade appears to have important therapeutic implications regarding both efficacy and safety. Blockade of one or the other, or both, can be done at the level of the cytokine subunits or the receptor subunits.

### Example 23

### Panning & Screening of Human Library 1-4

Phage generated from human library 1-4 were panned on recombinant human IL-23R/Fc chimera (R&D Systems), and recombinant mouse IL-23R/Fc chimera (R&D Systems). Screening of these binding panels after three, four, and/or five rounds of panning using an ELISA plate assay identified receptor-specific binders in all cases.

To generate phage for panning, the master library DNA was transformed by electroporation into bacterial strain TG1 (Stratagene). Cells were allowed to recover for one hour with shaking at 37° C. in SOC (Super-Optimal broth with Catabolite repression) medium prior to increasing the volume 10-fold by adding super broth (SB) to a final concentration of 20% glucose and 20 μg/mL carbenicillin. After shaking at 37° C. for one hour, the carbenicillin concentration was increased to 50 μg/mL for another hour, after which 400 mL of SB with 2% glucose and 50 μg/mL carbenicillin were added, along with helper phage M13K07 to a final concentration of 5×10⁹ pfu/mL. Incubation was continued at 37° C. without shaking for 30 minutes, and then with shaking at 100-150 rpm for another 30 min. Cells were centrifuged at 3200 g at 4° C. for 20 minutes, then resuspended in 500 mL SB medium containing 50 μg/mL carbenicillin and 50 μg/mL kanamycin. Cells were grown overnight at room temperature (RT) with shaking at 150 rpm. Phage were isolated by pelleting the bacterial cells by centrifugation at 15,000 g and 4° C. for 20 min. The supernatant was incubated with one-fourth volume (usually 250 mL of supernatant/bottle+62.5 mL PEG solution) of 20% PEG/2.5 M NaCl on ice for 30 min. The phage is pelleted by centrifugation at 15,000 g and 4° C. for 20 min. The phage pellet was resuspended in 1% bovine serum albumin (BSA) in phosphate buffered saline (PBS) containing 0.1% sodium azide (BSA/PBS/azide) and complete mini-EDTA-free protease inhibitors (Roche), prepared according to the manufacturer's instructions. Alternatively, phage was resuspended in Buffer D, containing 0.05% boiled cassein, 0.025% Tween-20, and protease inhibitors. Material was filter-sterilized using Whatman Puradisc 25 mm diameter, 0.2 μm pore size filters.

Phage generated from human library 1-4 were panned on recombinant human IL-23R/Fc chimera (R&D Systems cat #1686-MR). Library panning was performed either using a plate or a bead format. For the plate format, six to eight wells of a 96-well Immulon HB2 ELISA plate were coated with 250-1000 ng/well of carrier-free human IL-23R/Fc in Dulbecco's PBS. Material was incubated on the plate overnight, after which wells were washed three times with PBS, blocking buffer (either 1% BSA/PBS/azide or Buffer C, containing 0.05% boiled casseing and 1% Tween-20) was added, and wells were then incubated for at least 1 hour at 37° C. Additional wells were also treated with blocking buffer at the same time for later absorption of phage binding to blocking buffer.

Three dilutions of the phage preparation were used: undiluted, 1:10, and 1:100 in blocking buffer plus protease inhibitors. In some rounds of panning, recombinant human IgG1 Fc was added to each of the dilutions to a final concentration of 10 μg/mL. Blocking buffer was removed from the “Block Only” (preabsorption to block) wells and the different phage mixtures were incubated in these wells for another hour at 37° C. Aliquots (50 μL) of each phage mixture were transferred to a washed and blocked target well and allowed to incubate for 2 h at 37° C. For the first round of panning, bound phage were washed once with either 1×PBS/0.05% Tween or with Buffer D, and were eluted using glycine buffer, pH 2.2, containing 1 mg/mL BSA. After neutralization with 2 M Tris base (pH 11.5) the eluted phage were incubated for 15 minutes at room temperature with two to four milliliters of TG1 (Stratagene), XL1-Blue (Stratagene), ER2738 (Lucigen or NEB), or SS320 (Lucigen) cells at an optical density of approximately 0.9 measured at 600 nm (OD₆₀₀) in yeast extract-tryptone (YT) medium. Phage were prepared from this infection using the protocol above, but scaled down by about 20% (volume). Phage prepared from eluted phage were subjected to additional rounds of panning. At each round, titers of input and output phage were determined by plating on agar with appropriate antibiotics, and colonies from these plates were used later for screening for binders by ELISA.

Additional rounds of panning were performed as described above, except that in the second round of panning, washes were increased to 5×, and in subsequent rounds, washes were increased to 10×. Three to six rounds of panning were performed. For the final round of panning, phage were not produced after infection; rather, infected bacteria were grown overnight and a maxiprep (Qiagen kit) was prepared from the DNA. Glycerol stocks (15%) of input phage were stored frozen (at −80° C.) from each round.

For the bead panning format, human IL-23R was biotinylated and purified using a Sulfo-NHS micro biotinylation kit (Thermo-Scientific) according to the manufacturer's instructions. Phage were generated for panning from the master library as per the protocol above, except that the phage pellet was resuspended in a casein buffer containing 0.5% boiled casein, 0.025% Tween 20 in PBS with added EDTA-free protease inhibitors (Roche). Using a magnet, streptavidin magnetic beads (2 tubes with 50 μL or 0.5 mg each of Myone T1 Dynabeads (Invitrogen)) were washed several times in 0.5% boiled casein, 1% Tween 20 to remove preservatives. A 150 μL aliquot of the phage prep was preincubated with one tube of beads for 30 min at 37° C. to remove streptavidin binders. The phage prep was then removed from the beads and 1 μg of biotinylated IL-23R was added along with 10 μL of human Fc at 100 μg/mL and incubated for 2 h at 37° C. with rotation. This material was then added to the remaining tube of washed beads and incubated at 37° C. for 30 min. Using the magnetic stand, beads were washed five times with PBS/0.05% Tween. Phage were eluted with glycine, pH 2.0, neutralized, and used to infect bacteria as described above. In subsequent rounds of panning, bead-bound phage were washed ten times prior to elution. Titers of input and output phage were determined as described above.

For ELISA screening, colonies from later rounds of panning were grown in YT medium with 2% glucose and antibiotics overnight, and an aliquot of each was then used to start fresh cultures that were grown to an OD₆₀₀ of 0.5. Helper phage were added to 5×10⁹ pfu/mL and allowed to infect for 30 min at 37° C., followed by growth at 37° C. with agitation. Bacteria were centrifuged and resuspended in YT medium with carbenicillin and kanamycin and grown overnight for phage production. Bacteria were then pelleted and the medium was removed and mixed with one-fifth volume (1:5 milk mixture:supernatant) of 6×PBS, 18% milk. ELISA plates were prepared by incubating overnight at 4° C. with 50-100 μL of PBS containing 75-100 ng/well of recombinant human IL-23R/Fc. A duplicate plate coated with human IgG Fc (R&D Systems) was used as a control. Plates were washed 3 times with PBS, blocked for 1 h at 37° C. with 3% milk in 1×PBS, and incubated for 1 hour with 100 uL/well of each milk-treated phage mixture. Plates were washed once with PBS/0.05% Tween 20 and twice with PBS, incubated for one hour with an HRP-conjugated anti-M13 antibody (GE Healthcare), washed three times each with PBS/Tween and PBS, and incubated with TMB substrate (VWR). Sulfuric acid was added to stop the color reaction and absorbance was read at 450 nm to identify positive binders.

Binders to human IL-23R were identified from the third and fourth rounds of panning Examples of the sequences from the randomized regions of Loops 1 and 4 from phage-displayed CTLD binders to human IL-23R/Fc chimera are given in Table 11. Examination of these data suggests that for 31/36 of the binders, a motif was evident in the randomized region of Loop 4: the second and fifth amino acids were always glycine, the fourth amino acid was always one of the cyclic amino acids tryptophan or phenylalanine, the first amino acid was hydrophobic, and usually a cyclic amino acid, such as phenylalanine, tyrosine, or tryptophan, and the third amino acid was hydrophobic, and was usually valine. The Loop 1 region had less of a consensus, though glycine and serine appeared predominantly in the first and second positions, and valine was often in the seventh position. Five additional binders did not appear to have this consensus, though two of these probably formed another small group, with MFGMG (SEQ ID NO: 598) or LFGRG (SEQ ID NO: 599) in the Loop 4 region. Many binders were each represented by multiple clones.

TABLE 11
Sequences of human Loop 1 and 4 binders to
human IL-23R/Fc chimera

| | | Loop 1 | | Loop 4 |
|---|---|---|---|---|
| | Loop 1 | SEQ ID | Loop 4 | SEQ ID |
| Clone ID | Sequence | NO | Sequence | NO |
| 001-91.A1A | GSNVTQT | 376 | FGAFG | 377 |
| 001-91.Al2C | GSSVSDV | 378 | FGMWG | 379 |
| 001-69.4H1 | AGRYSLI | 380 | FGVFG | 381 |
| 001-69.4G8 | GSRRSGV | 382 | FGVFG | 381 |
| 001-69.3E5 | RGATVKV | 383 | FGVFG | 381 |
| 001-87.A8E | ANPAQDL | 384 | FGVWG | 385 |
| 001-89.C3G | APGAMEF | 386 | FGVWG | 385 |
| 001-89.C10B | GSPDLGV | 387 | FGVWG | 385 |
| 001-87.A5F | GSVRSAT | 388 | FGYFG | 389 |
| 001-91.A12E | GSPVGDM | 390 | IGVWG | 391 |
| 001-91.A7F | GSSKLGL | 392 | IGVWG | 391 |
| 001-69.4D4 | GSVRGRT | 393 | IGVWG | 391 |
| 001-69.3C2 | TNVTRTL | 394 | LGVWG | 395 |
| 001-87.A9E | GSALTNT | 396 | LGYWG | 395 |
| 001-89.C3C | ANRRRTM | 397 | MGVWG | 398 |
| 001-91.A7C | GSSVSGL | 399 | VGVFG | 400 |
| 001-69.4C6 | GSWLGDV | 401 | VGVFG | 400 |
| 001-89.C11E | SGKARDV | 402 | VGVFG | 400 |
| 001-91.A3D | GSRFGHL | 403 | WGVFG | 404 |
| 001-89.C3F | GSRISGV | 405 | WGVFG | 404 |
| 001-91.A6B | SGKRRTV | 406 | WGVFG | 404 |
| 001-89.C12C | SGSWART | 407 | WGVFG | 404 |
| 001-69.4C1 | AGARAEY | 408 | WGVWG | 409 |
| 001-69.4F2 | GPGQAGL | 410 | WGVWG | 409 |
| 001-91.A1B | GSTYTDL | 411 | WGVWG | 409 |
| 001-69.4G3 | GTRMTNT | 412 | WGYFG | 413 |
| 001-89.C7F | GSLLTGL | 414 | YGAWG | 415 |
| 001-69.3H4 | GSKAGKL | 416 | YGVFG | 417 |
| 001-69.4C12 | ASLRSRV | 418 | YGVWG | 419 |
| 001-69.4E5 | GNPSGSV | 420 | YGVWG | 419 |
| 001-87.A3B | TGALHQV | 421 | YGVWG | 419 |
| 001-89.C12E | WTKRTAL | 422 | MFGMG | 423 |
| 001-87.A4A | WTLAKNL | 424 | LFGRG | 425 |
| 001-69.4F5 | VLGWRRE | 426 | LVMPM | 427 |
| 001-69.3G5 | LATWLRW | 428 | QRMSY | 429 |
| 001-69.4F9 | QHLGSFW | 430 | VEFQG | 431 |

ELISA assays indicated that these binders did not cross-react with either human IgG1 Fc or with recombinant mouse IL-23R. ELISA and Biacore binding assays indicated that purified monomeric CTLD or full-length trimers from candidate clones 001-69.4G8 and other competed with IL-23 for binding to the human IL-23R. Competitive candidates have been identified that have nanomolar affinities.

An example of a sequence from the randomized regions of Loops 1 and 4 from phage-displayed CTLD binders to mouse IL-23R/Fc is given in Table 12. This sequence has similarity to the primary motif seen in the human IL-23R binders (compare Loop 1, for example, to B12C, or Loop 4 to C12C). Interestingly, the invariant cyclic tryptophan/phenylalanine of position 4 in Loop 4 was replaced with glycine in the mouse IL-23R binder.

TABLE 12
Sequences of human Loop 1 and 4
binders to mouse IL-23R

| | Loop 1 | Loop 4 |
|---|---|---|
| Clone | [SEQ ID NO] | [SEQ ID NO] |
| H1-4P141D | GSSQMDV [432] | WGLGG [433] |

### Example 24

### Affinity Maturation of Binders to Human IL-23R

Because the Loop 4 region of the human IL-23R appeared to be a relevant motif, a shuffling approach was developed preserving the diversity of Loop 4 regions already obtained by panning, but resorting them with all possible Loop 1 regions from the original naïve library. To this end, DNA from the round 4 panning of human IL-23R was digested with EcoRI and BssHII restriction enzymes, which cut between the Loop 1 and Loop 4 regions, and a fragment of about 1.4 kb, containing the Loop 4 region, was isolated. Separately, the original human 1-4 library DNA was digested with the same enzymes, and a fragment of about 3.5 kb, containing the Loop 1 region, was isolated. These fragments were ligated together and a new h1-4 shuffle library was generated as described above. The library was panned using the bead protocol (supra), except that at each round of panning the amount of biotinylated recombinant human IL-23R/Fc was decreased about 10-fold, from 200 ng, (to 20 ng, to 2 ng,) to 0.1 ng. Phage supernatants from colonies were screened by ELISA as described above and binders were identified and sequenced. Loop 1 and 4 sequences of the affinity-matured binders appear in Table 13.

TABLE 13
Loop 1 and 4 sequences from affinity-
matured human Loop 1-4 binders to
human IL-23R

| | | Loop 1 | | Loop 4 |
|---|---|---|---|---|
| | Loop 1 | SEQ ID | Loop 4 | SEQ ID |
| Clone | Sequence | NO | Sequence | NO |
| 056-40.A3C | GSATTAT | 434 | FGYFG | 389 |
| 056-45.F7F | GSATTDT | 435 | FGYFG | 389 |
| 056-41.B5C | GSALTNT | 396 | FGYFG | 389 |
| 056-53.H7H | GSSVSDV | 378 | FGYFG | 389 |
| 056-53.H4E | GSALTNT | 396 | FGVFG | 381 |
| 056-53.H1G | SGHWRAV | 436 | FGVFG | 381 |
| 056-42.C7D | GSNVTQT | 376 | YGVFG | 417 |
| 056-41.B12F | GSVRSAT | 388 | YGVFG | 417 |
| 056-41.B9B | APPDLGL | 437 | WGVWG | 409 |
| 056-42.C7F | APKSRQY | 438 | FGVWG | 385 |
| 056-44.E4G | VMQLPRK | 439 | IGVWG | 391 |
| 056-53.H7B | AGRMGLV | 440 | WGVFG | 404 |

A separate affinity maturation library was generated in which the diversity of the Loop 1 regions obtained in the initial panning round 4 was maintained, a limited selection of Loop 4 options was utilized, and Loop 3 was randomized in six positions. This was achieved by generating primers to amplify the Loop 1 region using DNA from the original panning round 4 of the human Loop 1-4 library as template, along with primers Bglfor (SEQ ID NO: 158) and H1-3-4R (SEQ ID NO: 185). This primer encodes the following amino acid sequence for loops 3 and 4:

(SEQ ID NO: 600)
RIAYKNWEXXXXXQPXGG(F/L)G(F/Y/V/D)(F/W/L/C)GENCAVL
S.

This sequence incorporates the primary alternatives for Loop 4, as well as alterations of the Loop 3 region of the CTLD. Other primers similar to this but more specific for the Loop 4 region sequences were also generated and used for production of another library randomized in the Loop 3 region. The remainder of the region of interest was generated by overlap PCR using primers PstLoop4rev (SEQ ID NO: 186) and Pst Rev (SEQ ID NO: 142).

Affinity matured IL-23R binding sequences obtained from these libraries are provided in Table 14. Some of the binders obtained were altered by swapping more favorable loop 4 or loop 1 sequences for others to obtain additional affinity-matured binders, and these are included in Table 14.

TABLE 14

| | | SEQ | | SEQ | | SEQ |
|---|---|---|---|---|---|---|
| | | ID | | ID | | ID |
| Clone name | Loop 1 | NO | Loop 3 | NO | Loop 4 | NO |
| H4EP1E9 | GSALTNT | 396 | AGYTKQPS | 441 | FGVFG | 381 |
| H4EWP1E9 | GSALTNT | 396 | AGYTKQPS | 441 | WGVFG | 404 |
| H4EP1E1 | GSALTNT | 396 | LLLRNQPP | 442 | FGVFG | 381 |
| H4EP1D6 | GSALTNT | 396 | QEPAKQPT | 443 | FGVFG | 381 |
| 101-51-1A10 | GSALTNT | 396 | HPLPPQPS | 444 | FGYFG | 389 |
| 101-51-1A3 | GSALTNT | 396 | HQPVYQPG | 445 | WGVFG | 404 |
| 101-54-4B3 | GSALTNT | 396 | LPPPGHPQ | 446 | FGVFG | 381 |
| 101-51-1A5 | GSALTNT | 396 | NGHEPQPR | 447 | FGYFG | 389 |
| 101-51-1A6 | GSALTNT | 396 | NNLSAQPR | 448 | FGYFG | 389 |
| 101-51-1A9 | GSALTNT | 396 | PARQPQPG | 449 | FGYFG | 389 |
| 101-80-5E8 | GSALTNT | 396 | PPEPLHPM | 450 | FGVFG | 381 |
| 101-54-4B6 | GSALTNT | 396 | PPGPHHPM | 451 | FGVFG | 381 |
| 101-113-6C108 | GSALTNT | 396 | PPPPHHPM | 452 | FGVFG | 381 |
| 101-51-1A4 | GSALTNT | 396 | RPALVQPR | 453 | FGVFG | 381 |
| 101-54-4B10 | GSALTNT | 396 | RPPLYQPG | 454 | FGYFG | 389 |
| 101-51-1A7 | GSALTNT | 396 | RPPLYQPG | 454 | WGVFG | 404 |
| 121-26-1A7F | GSALTNT | 396 | RPPLYQPG | 454 | FGVFG | 381 |
| 101-51-1A8 | GSALTNT | 396 | RTPPWQPE | 455 | FGYFG | 389 |
| 101-113-6C102 | GSNVTQT | 376 | PPPPHHPQ | 456 | FGVFG | 381 |
| 101-54-4Al2 | GSRRSGV | 382 | PPGPAHPQ | 457 | FGVFG | 381 |
| 101-113-6A44 | LAGWGMS | 458 | TPPRTQPP | 459 | FGVFG | 381 |
| 101-80-5H3* | GSALTNT | 396 | PPAPYHPM | 460 | -GVFG | 461 |

*Clone 101-80-5H3 had an amino acid deleted from the planned loop 4 and two other amino acid changes (GlyGly to AlaAla) in the loop 4 region just upstream of the altered region.

Table 15 shows some additional clones that were made with a primer similar to H1-3-4R (SEQ ID NO: 185), but having a coding sequences for the following loop modications.

TABLE 15

| | | SEQ | | SEQ | | SEQ |
|---|---|---|---|---|---|---|
| | | ID | | ID | | ID |
| Clone name | Loop 1 | NO | Loop 3 | NO | Loop 4 | NO |
| 079-86-P1D6h14 | GSTLTRI | 462 | QEPAKQPT | 443 | FGAFG | 377 |
| 079-71-P1E1 | GSALTNT | 396 | LLLRNQPP | 442 | FGAFG | 377 |
| 079-71-P1E9 | GSALTNT | 396 | AGYTKQPS | 441 | LGAFG | 463 |

Another affinity maturation library was generated by limiting loop 4 to five amino acid sequences: FGVFG (SEQ ID NO: 381), WGVFG (SEQ ID NO: 404), FGYFG (SEQ ID NO: 389), WGYFG (SEQ ID NO: 413), and WGVWG (SEQ ID NO: 409), while maintaining the GlySer found at the beginning of loop 1 in IL-23R binders, and varying the subsequent five amino acids in loop 1 using an NNK strategy. Primers GSXX (SEQ ID NO: 194) and 090827 BssBglrev (SEQ ID NO: 195) were mixed and extended using PCR, and primers FGVFGfor, FGYFGfor, WGVFGfor, WGYFGfor, and WGVWGfor (SEQ ID NOS: 196 to 200) were mixed individually with primer Pst Loop 4 rev (SEQ ID NO: 186) and extended using PCR. The resulting fragments were gel purified and mixed and extended by PCR in the presence of primers Bgl for (SEQ ID NO: 158) and Pst rev (SEQ ID NO: 142). The resulting fragments were digested with Bgl II and Pst I and inserted into vector pANA27 for phage display. Bead panning with successive target dilution was used to select affinity-matured candidates from the library. Sequences of the candidates obtained from this library are provided in Table 16.

TABLE 16

| | | SEQ ID | | SEQ ID |
|---|---|---|---|---|
| Candidate | LOOP 1 | NO: | LOOP 4 | NO: |
| 105-20-1H7 | GSAGTNT | 464 | FGYFG | 389 |
| 105-57-2E8 | GSAHTDT | 465 | WGYFG | 413 |
| 105-08-2G2 | GSAITDT | 466 | WGYFG | 413 |
| 105-08-2B3 | GSAITNT | 467 | WGYFG | 413 |
| 105-20-2C4a | GSAKTDT | 468 | WGYFG | 413 |
| 105-20-1A6 | GSAKTGT | 469 | WGYFG | 413 |
| 105-59-3E5 | GSAKTNT | 470 | WGYFG | 413 |
| 105-08-1C6 | GSALTDT | 471 | FGYFG | 389 |
| 105-08-1D1 | GSALTDT | 471 | WGYFG | 413 |
| 105-20-1B3 | GSALTNT | 396 | FGYFG | 389 |
| 105-59-3H6 | GSALTRT | 472 | WGVFG | 404 |
| 105-59-3C8 | GSALTSL | 473 | WGVWG | 409 |
| 105-57-2D11 | GSARGRV | 474 | WGVWG | 409 |
| 105-20-2F10 | GSARTDT | 475 | FGYFG | 389 |
| 105-08-2D2 | GSARTGT | 476 | FGYFG | 389 |
| 105-08-1D10 | GSARTGT | 476 | WGYFG | 413 |
| 105-08-1A4 | GSAVTNT | 477 | FGYFG | 389 |
| 105-08-2F6 | GSAYTNT | 478 | FGYFG | 389 |
| 105-08-2E12 | GSGLTDT | 479 | WGYFG | 413 |
| 105-55-1A10 | GSGWTGL | 480 | WGVWG | 409 |
| 105-20-2F12 | GSKLTDT | 481 | FGYFG | 389 |
| 105-82-4A3 | GSKVSGL | 482 | WGVFG | 404 |
| 105-08-1D3 | GSKVTET | 483 | FGYFG | 389 |
| 105-61-4D8 | GSLKTDT | 484 | FGVFG | 381 |
| 105-08-2C11 | GSLKTQT | 485 | WGYFG | 413 |
| 105-08-2C10 | GSLLTDT | 486 | FGVFG | 381 |
| 105-08-2G6 | GSLLTDT | 486 | WGYFG | 413 |
| 105-59-3A5 | GSLLTNT | 487 | FGVFG | 381 |
| 105-08-2C4 | GSLLTNT | 487 | FGYFG | 389 |
| 105-61-4B2 | GSLRSDL | 488 | FGVFG | 381 |
| 105-61-4G3 | GSLRTDT | 489 | FGVFG | 381 |
| 105-08-1G12 | GSLRTGT | 490 | WGYFG | 413 |
| 105-78-2D1 | GSLRTHT | 491 | FGVFG | 381 |
| 105-78-2E6 | GSLRTNT | 492 | FGVFG | 381 |
| 105-59-3B9 | GSMLTDT | 493 | FGVFG | 381 |
| 105-08-2A1 | GSMRTDT | 494 | WGYFG | 413 |
| 105-08-2H10 | GSNHTDT | 495 | FGYFG | 389 |
| 105-59-3B5 | GSPITDT | 496 | FGVFG | 381 |
| 105-20-2A3 | GSPITNT | 497 | FGYFG | 389 |
| 105-08-1G9 | GSPKTDT | 498 | FGYFG | 389 |
| 105-08-2G7 | GSPKTGT | 499 | FGYFG | 389 |
| 105-08-2G1 | GSPKTHT | 500 | FGYFG | 389 |
| 105-08-2G10 | GSPLTDT | 501 | FGYFG | 389 |
| 105-61-4G5 | GSPLTNT | 502 | FGVFG | 381 |
| 105-20-1H1 | GSPLTNT | 502 | WGYFG | 413 |
| 105-08-1B7 | GSPRTDT | 503 | FGYFG | 389 |
| 105-08-1A3 | GSPRTDT | 503 | WGVFG | 404 |
| 104-101-1A3F | GSPRTDT | 503 | FGVFG | 381 |
| 105-08-2H11 | GSPRTDT | 503 | WGYFG | 413 |
| 105-08-2H12 | GSPRTET | 504 | FGYFG | 389 |
| 105-08-2G4 | GSPRTGT | 505 | FGYFG | 389 |
| 105-59-3D6 | GSPRTHT | 506 | FGYFG | 389 |
| 105-08-1A8 | GSPRTNT | 507 | FGVFG | 381 |
| 105-20-2G12 | GSPRTNT | 507 | FGYFG | 389 |
| 105-08-1B1 | GSPRTQT | 508 | FGYFG | 389 |
| 105-57-2E11 | GSPRTSV | 509 | FGYFG | 389 |
| 105-08-2H2 | GSPTTDT | 510 | WGYFG | 413 |
| 105-59-3C11 | GSPVNDV | 511 | FGYFG | 389 |
| 105-08-1D2 | GSPVTDT | 512 | FGYFG | 389 |
| 105-55-1F3 | GSPVTDT | 512 | WGYFG | 413 |
| 105-08-2H6 | GSPVTGT | 513 | FGYFG | 389 |
| 105-59-3F1 | GSPVTNT | 514 | FGYFG | 389 |
| 105-59-3H4 | GSQLTDT | 515 | FGYFG | 389 |
| 105-08-1C3 | GSQLTDT | 515 | WGYFG | 413 |
| 105-57-2E2 | GSQLTNT | 516 | FGYFG | 389 |
| 105-08-2C12 | GSQRTDT | 517 | FGYFG | 389 |
| 105-08-2C6 | GSQRTDT | 517 | WGYFG | 413 |
| 105-08-1C2 | GSRATDT | 518 | FGYFG | 389 |
| 105-08-1B10 | GSRHTDT | 519 | FGYFG | 389 |
| 105-76-1D11 | GSRLTDT | 520 | WGVFG | 404 |
| 105-59-3E3 | GSRLTNT | 521 | FGYFG | 389 |
| 105-55-1E3 | GSRRTDT | 522 | FGYFG | 389 |
| 105-20-2G5 | GSRRTDT | 522 | WGYFG | 413 |
| 105-08-1A10 | GSSITDT | 523 | WGYFG | 413 |
| 105-08-1G2 | GSSKTNT | 524 | WGYFG | 413 |
| 105-59-3F9 | GSSLTDT | 525 | FGYFG | 389 |
| 105-08-2C1 | GSSLTDT | 525 | WGYFG | 413 |
| 105-61-4H2 | GSSLTNT | 526 | FGYFG | 389 |
| 105-08-2H3 | GSSLTNT | 526 | WGYFG | 413 |
| 105-08-1C11 | GSSRTDT | 527 | FGYFG | 389 |
| 105-20-1B4 | GSSRTNT | 528 | WGYFG | 413 |
| 105-08-1C10 | GSSVTNT | 529 | WGYFG | 413 |
| 105-82-4A11 | GSSVTST | 530 | WGVFG | 404 |
| 105-08-1C9 | GSTLTDT | 531 | FGYFG | 389 |
| 105-08-1C4 | GSTLTDT | 531 | WGYFG | 413 |
| 105-59-3G12 | GSTLTNT | 532 | FGYFG | 389 |
| 105-08-2C9 | GSTLTNT | 532 | WGYFG | 413 |
| 105-55-1All | GSTMTQT | 533 | FGYFG | 389 |
| 105-59-3G9 | GSTRTDT | 534 | FGYFG | 389 |
| 105-59-3B11 | GSTRTNT | 535 | FGYFG | 389 |
| 105-61-4B12 | GSVITGT | 536 | FGYFG | 389 |
| 105-61-4E5 | GSVITNT | 537 | FGYFG | 389 |
| 105-20-2C4b | GSVKTDT | 538 | WGYFG | 413 |
| 105-08-1D12 | GSVLTDT | 539 | FGYFG | 389 |
| 105-59-3A6 | GSVLTGT | 540 | FGYFG | 389 |
| 105-55-1B9 | GSVLTNT | 541 | FGYFG | 389 |
| 105-08-2H4 | GSVRTDT | 542 | FGYFG | 389 |
| 105-80-3G12 | GSVRTDT | 542 | WGVFG | 404 |
| 105-20-2Cl1 | GSVRTDT | 542 | WGYFG | 413 |
| 105-80-3D4 | GSVRTES | 543 | FGVFG | 381 |
| 105-59-3F11 | GSVRTGT | 544 | FGYFG | 389 |
| 105-08-1A7 | GSVRTNT | 545 | FGYFG | 389 |
| 105-20-2C7 | GSVTTDT | 546 | FGYFG | 389 |
| 105-57-2H2 | GSWGSGI | 547 | WGVWG | 409 |
| 105-08-2C8 | GSWLTDT | 548 | WGYFG | 413 |
| 105-55-1D12 | GSYLTNT | 549 | FGYFG | 389 |

Additional changes in the amino acid sequences of the loops and surrounding sequences were generated by alanine scanning, i.e. the replacement of specific amino acids with the amino acid alanine by means of gene site specific mutagenesis, known to those skilled in the art. Table 17 describes the alanine replacements made in the candidate 056-53.H4E sequence. Such replacements are not limited to the residues shown and can be made in any candidate backbone. Table 17 shows that many of these replacements were beneficial for affinity and/or protein production.

TABLE 17
Sequences of alanine scan candidates that bind IL-23R.

| | | SEQ |
|---|---|---|
| | | ID |
| Candidate | Sequence of AA 115 to 172* | NO. |
| 056-53.H4E | NGSALTNTWVDMTGARIAYKNWETEITAQPDGGFGVFGENCAVLSGAANGKWFDKRCR | 550 |
| H4E N115A | AGSALTNTWVDMTGARIAYKNWETEITAQPDGGFGVFGENCAVLSGAANGKWFDKRCR | 551 |
| H4E G116A | NASALTNTWVDMTGARIAYKNWETEITAQPDGGFGVFGENCAVLSGAANGKWFDKRCR | 552 |
| H4E S117A | NGAALTNTWVDMTGARIAYKNWETEITAQPDGGFGVFGENCAVLSGAANGKWFDKRCR | 553 |
| H4E L119A | NGSAATNTWVDMTGARIAYKNWETEITAQPDGGFGVFGENCAVLSGAANGKWFDKRCR | 554 |
| H4E T120A | NGSALANTWVDMTGARIAYKNWETEITAQPDGGFGVFGENCAVLSGAANGKWFDKRCR | 555 |
| H4E N121A | NGSALTATWVDMTGARIAYKNWETEITAQPDGGFGVFGENCAVLSGAANGKWFDKRCR | 556 |
| H4E T122A | NGSALTNAWVDMTGARIAYKNWETEITAQPDGGFGVFGENCAVLSGAANGKWFDKRCR | 557 |
| H4E W123A | NGSALTNTAVDMTGARIAYKNWETEITAQPDGGFGVFGENCAVLSGAANGKWFDKRCR | 558 |
| H4E R130A | NGSALTNTWVDMTGAAIAYKNWETEITAQPDGGFGVFGENCAVLSGAANGKWFDKRCR | 559 |
| H4E K134A | NGSALTNTWVDMTGARIAYANWETEITAQPDGGFGVFGENCAVLSGAANGKWFDKRCR | 560 |
| H4E N135A | NGSALTNTWVDMTGARIAYKAWETEITAQPDGGFGVFGENCAVLSGAANGKWFDKRCR | 561 |
| H4E W136A | NGSALTNTWVDMTGARIAYKNAETEITAQPDGGFGVFGENCAVLSGAANGKWFDKRCR | 562 |
| H4E E137A | NGSALTNTWVDMTGARIAYKNWATEITAQPDGGFGVFGENCAVLSGAANGKWFDKRCR | 563 |
| H4E T138A | NGSALTNTWVDMTGARIAYKNWEAEITAQPDGGFGVFGENCAVLSGAANGKWFDKRCR | 564 |
| H4E E139A | NGSALTNTWVDMTGARIAYKNWETAITAQPDGGFGVFGENCAVLSGAANGKWFDKRCR | 565 |
| H4E I140A | NGSALTNTWVDMTGARIAYKNWETEATAQPDGGFGVFGENCAVLSGAANGKWFDKRCR | 566 |
| H4E T141A | NGSALTNTWVDMTGARIAYKNWETEIAAQPDGGFGVFGENCAVLSGAANGKWFDKRCR | 567 |
| H4E Q143A | NGSALTNTWVDMTGARIAYKNWETEITAAPDGGFGVFGENCAVLSGAANGKWFDKRCR | 568 |
| H4E D145A | NGSALTNTWVDMTGARIAYKNWETEITAQPAGGFGVFGENCAVLSGAANGKWFDKRCR | 569 |
| H4E G146A | NGSALTNTWVDMTGARIAYKNWETEITAQPDAGFGVFGENCAVLSGAANGKWFDKRCR | 570 |
| H4E G147A | NGSALTNTWVDMTGARIAYKNWETEITAQPDGAFGVFGENCAVLSGAANGKWFDKRCR | 571 |
| H4E E153A* | NGSALTNTWVDMTGARIAYKNWETEITAQPDGGFGVFGANCAVLSGAANGKWFDKRCR | 572 |
| H4E N154A* | NGSALTNTWVDMTGARIAYKNWETEITAQPDGGFGVFGEACAVLSGAANGKWFDKRCR | 573 |
| H4E R170A* | NGSALTNTWVDMTGARIAYKNWETEITAQPDGGFGVFGENCAVLSGAANGKWFDKACR | 574 |
| H4E R172A* | NGSALTNTWVDMTGARIAYKNWETEITAQPDGGFGVFGENCAVLSGAANGKWFDKRCA | 575 |

*Note that the numbering of 056-53.H4E amino acids diverges from the TN sequence numbering in the last four candidates listed, because of the introduction in loop 4 of three additional amino acids. Thus E153 in 056-53.H4E corresponds to E150 in the original TN sequence (FIG. 2, for example). Which figure does this go with?

TABLE 18
Affinity and production level in E. coli periplasm of 056-53.H4E
ATRIMER ™ polypeptide complexes generated by alanine scanning

| Atrimer | K_{D}(nM) | mg/L |
|---|---|---|
| 056-53.H4E | 0.772 | 1.430 |
| H4E N115A | 7.560 | 0.923 |
| H4E G116A | 10.700 | 1.680 |
| H4E S117A | 2.230 | 1.314 |
| H4E L119A | 1.330 | 1.600 |
| H4E T120A | 1.210 | 1.500 |
| H4E N121A | 0.989 | 1.100 |
| H4E T122A | 6.690 | 1.000 |
| H4E W123A | 11.500 | 1.100 |
| H4E R130A | 1.570 | 1.940 |
| H4E K134A | 1.580 | 0.764 |
| H4E N135A | 1.170 | 0.546 |
| H4E W136A | 14.400 | 0.484 |
| H4E E137A | 0.597 | 1.850 |
| H4E T138A | 0.743 | 2.218 |
| H4E E139A | 0.640 | 1.194 |
| H4E I140A | 1.280 | 1.706 |
| H4E T141A | 0.651 | 1.378 |
| H4E Q143A | 0.689 | 0.444 |
| H4E D145A | 0.714 | 0.876 |
| H4E G146A | 0.960 | 1.092 |
| H4E G147A | 1.030 | 0.512 |
| H4E E153A* | 0.948 | 0.750 |
| H4E N154A* | 0.843 | 1.570 |
| H4E R170A* | 0.777 | 1.984 |
| H4E R172A* | 1.080 | 0.836 |

### Example 25

Subcloning and Production of CTLD and Atrimer™ Polypeptide Complex Binders to Human IL-23R

The DNA fragments encoding loop regions were obtained by restriction digestion with BglII and PstI (or MfeI) restriction enzymes, and ligated to the bacterial CTLD expression vectors pANA1 (SEQ ID NO: 51), pANA3 (SEQ ID NO: 53), or pANA12 (SEQ ID NO: 62) that were pre-digested with BglII and PstI. pANA1 is a T7 based expression vector designed to express C-terminal 6×His-tagged human monomeric CTLD. The pelB signal peptide directs the proteins to the periplasm or growth medium. pANA3 is the C-terminal HA-His-tagged version of pANA1. pANA12 is the C-terminal HA-StrepII-tagged version of pANA1. For expression of trimeric protein, the loop regions can be sub-cloned into ATRIMER™ polypeptide complex expression vectors pANA4 (SEQ ID NO: 54) or pANA10 (SEQ ID NO: 60) to produce secreted ATRIMER™ polypeptide complexes in E. coli. pANA4 is a pBAD based expression vector containing C-terminal His/Myc-tagged full length human TN with an ompA signal peptide to direct the proteins to periplasm or growth medium. pANA10 is the C-terminal HA-StrepII-tagged version of pANA4.

The expression constructs were transformed into E. coli strains BL21(DE3). Star (for pANA1, pANA3 and pANA12; monomeric CTLD production) or BL21(DE3) (for pANA4 and pANA10; ATRIMER™ polypeptide copmlexproduction) were plated on LB/agar plates with appropriate antibiotics. A single colony on a fresh plate was inoculated into 1 L of either SB with 1% glucose and kanamycin (for pANA1 and pANA12 vectors) or 2×YT (doubly concentrated yeast tryptone) medium with ampicillin (for pANA4 and pANA10 vectors). The cultures were incubated at 37° C. on a shaker at 200 rpm to an OD₆₀₀ of 0.5, then cooled to room temperature. IPTG was added to a final concentration of 0.05 mM for pANA1 and pANA12, while arabinosis was added to a final concentration of 0.002-0.02% for pANA4 and pANA10. The induction was performed overnight at room temperature with shaking at 120-150 rpm, after which the bacteria were collected by centrifugation. The periplasmic proteins were extracted by osmotic shock or gentle sonication.

The 6×His-tagged proteins were purified using Ni⁺-NTA affinity chromatography. Briefly, periplasmic proteins were reconstituted in a His-binding buffer (100 mM HEPES, pH 8.0, 500 mM NaCl, 10 mM imidazole) and loaded onto a Ni⁺-NTA column pre-equilibrated with His-binding buffer. The column was washed with 10× volume of binding buffer. The bound proteins were eluted with an elution buffer (100 mM HEPES, pH 8.0, 500 mM NaCl, 500 mM imidazole). The purified proteins were dialyzed into 1×PBS buffer and bacterial endotoxin was removed by anion exchange.

The strep II-tagged monomeric CTLDs and ATRIMER™ polypeptide complexes were purified by Strep-Tactin affinity chromatography. Briefly, periplasmic proteins were reconstituted in 1×PBS buffer and loaded onto a Strep-Tactin column pre-equivalent with 1×PBS buffer. The column was washed with 10× volume of PBS buffer. The proteins were eluted with elution buffer (1×PBS with 2.5 mM desthiobiotin). The purified proteins were dialyzed into 1×PBS buffer and bacterial endotoxin was removed by anion exchange.

For some cell assays, ATRIMER™ polypeptide complexes were produced by mammalian cells. DNA fragments encoding loop regions were sub-cloned into the mammalian expression vector pANA2 or pANA11 to produce ATRIMER™ polypeptide complexes in the HEK293 transient expression system. pANA2 is a modified pCEP4 vector containing a C-terminal His tag. pANA11 is the C-terminal HA-StrepII-tagged version of pANA2. The DNA fragments encoding loop region were obtained by double digestion with Bgl II and MfeI and ligated into the expression vectors pANA2 and pANA11 pre-digested with Bgl II and MfeI. The expression plasmids were purified from bacteria using a Qiagen HiSpeed Plasmid Maxi Kit (Qiagene). For HEK293 adhesion cells, transient transfection was performed using Qiagen SuperFect Reagent according to the manufacturer's protocol. The day after transfection, the medium was removed and changed to 293 Isopro serum-free medium (Irvine Scientific). Two days later, glucose in 0.5 M HEPES buffer was added into the media to a final concentration of 1%. The tissue culture supernatant was collected 4-7 days after transfection for purification. For HEK 293F suspension cells, the transient transfection was performed by Invitrogen's 293Fectin according to the manufacturer's protocol. The next day, 1× volume of fresh medium was added into the culture. The tissue culture supernatant was collected 4-7 days after transfection for purification.

The His or Strep II-tagged ATRIMER™ polypeptide complex purification from mammalian tissue culture supernatant was performed as described for E. coli produced ATRIMER™ polypeptide complexes.

### Example 26

### Characterization of Binders by ELISA and Competition ELISA

ELISA assays, performed as described in Example 23, demonstrated that none of the phage-displayed binders cross-reacted with either human IgG1 Fc or with recombinant mouse IL-23R/Fc (R&D Systems).

Competitive ELISA assays were performed using purified monomeric CTLDs or ATRIMER™ polypeptide complexes generated as described above from positive human IL-23R (IL-23R) binders to block binding of human IL-23 to human IL-23R. Assays were performed generally as follows. Individual wells in Immulon HB2 plates were incubated overnight at 4° C. with 100 μL PBS containing 100 ng of an anti-human IgG Fc (R&D MAB 110 clone 97924). Plates were washed five times with PBS/0.05% Tween 20, and wells were incubated for 1.5 h at RT with 100 μL each of PBS containing 50 ng of recombinant human IL-23R/Fc. Plates were washed as before and blocked for 1 h at RT with 150 μL of 3% bovine serum albumin (Sigma) in PBS, after which plates were washed as described, and wells were incubated for 1-2 hours at RT with 100 μL each of PBS containing IL-23 with or without competitor (ATRIMER™ polypeptide copmlexor CTLD). IL-23-containing solutions were prepared as follows. Human IL-23 (eBioscience) was added at a concentration of 100 ng/mL. Competitor was included at a final concentration of 1 μg/mL. After incubation, plates were washed as described and wells were incubated for 40 min at RT with 100 μL each of PBS containing a 1:5000 dilution of streptavidin-HRP conjugate (Pierce catalog no. 21130). After washing, wells were incubated with 100 μL each of TMB (BioFX Lab catalog no. TMBH-1000-0) for up to 30 min at RT. Reactions were stopped with an equal volume of 0.2 M sulfuric acid.

An example of the results of the competition assay (inhibiting IL-23/IL-23R interaction) using the ATRIMER™ polypeptide complexes from the initial panning is presented in FIG. 11. ATRIMER™ polypeptide complexes to the left of the wild-type human tetranectin control (TN) were obtained from the third round of panning against human IL-23R using the human Loop 1-4 library (except for P1D1). ATRIMER™ polypeptide complexes to the right of the tetranectin control were obtained from the human 1-4 shuffle library after 3-4 rounds of panning on decreasing quantities of IL-23R. The ability of candidate molecules from the affinity-matured panning procedure to compete with IL-23 binding to IL-23R is improved over that of candidates from the initial panning procedure.

A number of ATRIMER™ polypeptide complexes were tested in competition ELISA more extensively to determine IC50 values. As shown in Table 19, ATRIMER™ polypeptide complexes displayed low to subnanomolar IC50s.

TABLE 19
Ability of ATRIMER ™ polypeptide complexes to compete
with IL-23 for binding to IL-23R.

| | SEQ ID NOS of | Average IC50 |
|---|---|---|
| hIL-23R binder | Loops 1 & 4: | (nM) |
| H7H | 378, 389 | 0.53 |
| H7B | 440, 404 | 0.9 |
| 4G8 | 382, 381 | 1.4 |
| F7F | 435, 389 | 1.45 |
| B5C | 396, 389 | 1.65 |
| A3C | 434, 389 | 1.8 |
| 056-53.H4E | 396, 381 | 2.5 |
| A9E | 396, 395 | 2.6 |
| H1G | 436, 381 | 3.75 |

The ATRIMER™ polypeptide complex 056-53.H4E was chosen as a standard for comparison, and additional competition assays were performed with affinity-matured ATRIMER™ polypeptide complexes. Table 20 provides the ratio of the IC50 of tested ATRIMER™ polypeptide complexes to that of 056-53.H4E performed in the same assay, in order to better compare competition results among assays.

TABLE 20
Comparison of the ability of ATRIMER ™ polypeptide
complexes to compete with IL-23 for binding to IL-23R.

| | Ratio IC50 to |
|---|---|
| Atrimer | 056-53.H4E IC50 |
| 101-54-4B6 | 0.3 |
| 105-08 1D3 | 0.4 |
| 101-80-5E8 | 0.6 |
| H4E E137A | 0.8 |
| 105-59-3B5 | 0.8 |
| 105-61-4G3 | 0.8 |
| 105-08 2C10 | 0.9 |
| 101-113-6C108 | 0.9 |
| H4E T138A | 1.0 |
| 105-78-2E6 | 1.0 |
| 101-51-1A7 | 1.0 |
| 101-51-1A4 | 1.0 |
| 101-51-1A5 | 1.0 |
| 105-20 2G12 | 1.0 |
| 105-61-4G5 | 1.0 |
| 101-54-4B3 | 1.0 |
| 105-08 1A3 | 1.1 |
| 101-54-4A12 | 1.1 |
| 105-59-3A5 | 1.2 |
| H4E E139A | 1.2 |
| 105-20 2A3 | 1.2 |
| 105-20 1B3 | 1.2 |
| H4E D145A | 1.3 |
| 105-78-2D1 | 1.3 |
| H4E T141A | 1.4 |
| 101-54-4B10 | 1.4 |
| H4E R170A | 1.4 |
| 105-08 1A8 | 1.6 |
| 105-08 1A4 | 1.6 |
| 101-51-1A3 | 1.6 |
| H4E Q143A | 1.6 |
| 105-20 1H1 | 1.8 |
| 105-08 2G10 | 1.8 |
| H4E N154A | 1.9 |
| 101-113-6C102 | 2.0 |
| 105-08 1C6 | 2.0 |
| 105-20 1F3b | 2.0 |
| 105-08 2H6 | 2.0 |
| 105-20 1H7 | 2.1 |
| 101-51-1A9 | 2.2 |
| 105-08 2G1 | 2.2 |
| 105-08 2F6 | 2.4 |
| 105-08 1G9 | 2.4 |
| 105-20 1F3a | 2.5 |
| 105-08 2G7 | 2.5 |
| 105-08 2G4 | 2.5 |
| 101-51-1A6 | 2.6 |
| 105-08 1C11 | 2.8 |
| 105-20 2F12 | 2.8 |
| 105-20 2C4a | 2.9 |
| 105-08 1A7 | 2.9 |
| 105-08 2H3 | 2.9 |
| 105-08 2C4 | 2.9 |
| 105-20 1B4 | 3.0 |
| 105-08 1B1 | 3.3 |
| 105-08 2C12 | 3.3 |
| 105-08 2H12 | 3.3 |
| 105-08 1C4 | 3.3 |
| 105-08 2B3 | 3.4 |
| 105-20 2C7 | 3.5 |
| 105-08 1D1 | 3.6 |
| 105-08 2C1 | 3.6 |
| 105-08 1C3 | 3.6 |
| 105-08 2C6 | 3.6 |
| 101-51-1A8 | 3.7 |
| 105-08 2G2 | 3.8 |
| 105-08 2H2 | 4.0 |
| 105-08 1C2 | 4.1 |
| 105-08 1B7 | 4.1 |
| 105-08 2D2 | 4.1 |
| 105-20 2C4b | 4.2 |
| 105-20 2F10 | 4.2 |
| 105-08 1A10 | 4.3 |
| 105-08 1D2 | 4.3 |
| 105-08 2H11 | 4.3 |
| 105-08 1D12 | 4.6 |
| 105-08 1B10 | 4.7 |
| 105-20 2C11 | 4.8 |
| 105-08 1C10 | 5.0 |
| 105-08 2A1 | 5.0 |
| 105-08 2H4 | 5.0 |
| 105-08 2G6 | 5.2 |
| 105-08 2C9 | 5.3 |
| 105-20 2G5 | 5.3 |
| 105-08 1D10 | 5.5 |
| 105-08 1G2 | 5.5 |
| 105-08 2H10 | 6.5 |
| 105-20 1A6 | 6.6 |
| 105-08 1C9 | 7.4 |
| 105-08 2C8 | 8.4 |
| 101-51-1A10 | 8.7 |
| 105-08 2C11 | 9.1 |
| 105-08 2E12 | 9.1 |
| 101-80-5H3 | 11.3 |
| 105-08 1G12 | 13.2 |

### Example 27

### Characterization of the Affinity of Human IL-23R Binders by Biacore

Apparent affinities of the monomeric and trimeric binders from both the original library panning and the affinity matured library pannings are provided in Tables 21, 22 and 23. A Biacore 3000 biosensor (GE Healthcare) was used to evaluate the interaction of human IL-23R and receptor binders. Immobilization of an anti-human IgG Fc antibody (GE Heathcare) to the CM5 chip (Biacore) was performed using standard amine coupling chemistry, and this modified surface was used to capture a recombinant human IL-23R/Fc fusion protein (R&D Systems). A low-density receptor surface, less than 200 RU, was used for all of the analyses. ATRIMER™ polypeptide complex dilutions (1-500 nM) were injected over the IL-23R surface at 30 μl/min and kinetic constants were derived from the sensorgram data using the Biaevaluation software (version 3.1, GE Healthcare). Data collection was 3 minutes for the association and 5 minutes for dissociation. The anti-human IgG surface was regenerated with a 30 s pulse of 3M magnesium chloride. All sensorgrams were double-referenced against an activated and blocked flow-cell as well as buffer injections.

TABLE 21
Affinities of monomeric CTLD IL-23R binders from H Loop 1-4 library

| Analyte | Kₐ (1/M · s) | K_{d} (1/s) | K_{A} (1/M) | K_{D} (nM) |
|---|---|---|---|---|
| A5F | 1.70E+05 | 4.15E−03 | 4.11E+07 | 24.3 |
| 4G8 | 1.43E+05 | 7.83E−03 | 1.83E+07 | 54 |
| B1B | 1.15E+05 | 6.46E−03 | 1.77E+07 | 56.4 |
| A9E | 3.81E+04 | 4.10E−03 | 9.29E+06 | 108 |
| A8E | 5.37E+04 | 7.57E−03 | 7.09E+06 | 141 |
| 4D4 | 2.83E+04 | 4.19E−03 | 6.76E+06 | 148 |
| C7F | 3.58E+04 | 5.31E−03 | 6.75E+06 | 148 |
| C12E | 4.16E+04 | 7.40E−03 | 5.62E+06 | 178 |
| 3C2 | 3.99E+04 | 7.41E−03 | 5.39E+06 | 186 |
| C3C | 8.45E+04 | 1.58E−02 | 5.34E+06 | 187 |
| A4A | 1.18E+05 | 2.29E−02 | 5.18E+06 | 193 |
| 4F5 | 2.35E+04 | 5.71E−03 | 4.12E+06 | 243 |
| B1A | 2.18E+04 | 7.04E−03 | 3.09E+06 | 324 |
| 4E5 | 4.54E+04 | 1.61E−02 | 2.82E+06 | 355 |
| B12C | 1.26E+05 | 5.72E−02 | 2.20E+06 | 455 |
| B7C | 3.03E+04 | 1.99E−02 | 1.52E+06 | 656 |

TABLE 22
Affinities of full-length ATRIMER ™ polypeptide complex IL-23R
binders from the original and the first affinity-matured library.

| Analyte | Kₐ (1/M · s) | K_{d} (1/s) | K_{A} (1/M) | K_{D} (nM) |
|---|---|---|---|---|
| H7B | 4.31E+05 | 2.40E−04 | 1.80E+09 | 0.557 |
| B5C | 3.07E+05 | 3.14E−04 | 9.78E+08 | 1.02 |
| 056-53.H4E | 2.66E+05 | 3.14E−04 | 8.47E+08 | 1.18 |
| F7F | 2.98E+05 | 3.76E−04 | 7.92E+08 | 1.26 |
| H7H | 2.56E+05 | 3.85E−04 | 6.65E+08 | 1.5 |
| A3C | 2.13E+05 | 3.73E−04 | 5.70E+08 | 1.75 |
| A9E | 1.72E+05 | 3.30E−04 | 5.21E+08 | 1.92 |
| B12F | 2.44E+05 | 5.45E−04 | 4.47E+08 | 2.24 |
| A5F | 1.53E+05 | 7.00E−04 | 2.19E+08 | 4.57 |
| 4G8 m | 1.58E+05 | 7.51E−04 | 2.10E+08 | 4.76 |
| H1G | 9.52E+04 | 4.89E−04 | 1.95E+08 | 5.13 |
| B9B | 9.28E+04 | 4.78E−04 | 1.94E+08 | 5.15 |
| C7F | 7.22E+04 | 4.65E−04 | 1.55E+08 | 6.44 |
| 4G8 | 1.09E+05 | 8.05E−04 | 1.35E+08 | 7.42 |
| A4A | 5.06E+04 | 4.09E−04 | 1.24E+08 | 8.08 |
| C3C | 5.79E+04 | 4.83E−04 | 1.20E+08 | 8.34 |
| C6H | 4.95E+04 | 8.45E−04 | 5.85E+07 | 17.1 |

“4G8 TN m” refers to mammalian-cell produced material.
All other material was produced in E. coli.

TABLE 23
Affinities of ATRIMER ™ polypeptide complex IL-23R binders from
additional affinity-matured libraries and alanine-scan candidates.
All material was produced in E. coli.

| Analyte | Kₐ (1/M · s) | K_{d} (1/s) | K_{A} (1/M) | K_{D} (nM) |
|---|---|---|---|---|
| 101-113-6C102 | 2.71E+05 | 2.83E−04 | 9.62E+08 | 1.04 |
| 101-113-6C108 | 6.23E+05 | 3.82E−04 | 1.63E+09 | 0.613 |
| 101-51-1A10 | 1.67E+05 | 3.45E−04 | 4.85E+08 | 2.06 |
| 101-51-1A3 | 4.63E+05 | 2.62E−04 | 1.77E+09 | 0.565 |
| 101-51-1A4 | 1.02E+06 | 3.95E−04 | 2.58E+09 | 0.388 |
| 101-51-1A5 | 4.95E+05 | 2.89E−04 | 1.71E+09 | 0.584 |
| 101-51-1A6 | 5.57E+05 | 4.15E−04 | 1.34E+09 | 0.746 |
| 101-51-1A7 | 4.19E+05 | 1.87E−04 | 2.24E+09 | 0.447 |
| 101-51-1A8 | 2.62E+05 | 3.96E−04 | 6.62E+08 | 1.51 |
| 101-51-1A9 | 3.45E+05 | 3.29E−04 | 1.05E+09 | 0.955 |
| 101-54-4A12 | 1.24E+06 | 5.73E−04 | 2.16E+09 | 0.463 |
| 101-54-4B10 | 4.79E+05 | 4.29E−04 | 1.11E+09 | 0.897 |
| 101-54-4B3 | 1.13E+06 | 3.64E−04 | 3.12E+09 | 0.321 |
| 101-54-4B6 | 6.87E+05 | 3.90E−04 | 1.76E+09 | 0.569 |
| 101-80-5E8 | 1.13E+06 | 3.91E−04 | 2.89E+09 | 0.346 |
| 101-80-5H3 | 5.05E+04 | 3.27E−04 | 1.55E+08 | 6.46 |
| 105-08 1A3 | 7.35E+05 | 3.48E−04 | 2.11E+09 | 0.473 |
| 105-08 1A4 | 2.50E+05 | 3.12E−04 | 8.00E+08 | 1.250 |
| 105-08 1A8 | 7.37E+05 | 3.44E−04 | 2.14E+09 | 0.467 |
| 105-08 1D3 | 2.28E+05 | 3.01E−04 | 7.58E+08 | 1.320 |
| 105-08 2C10 | 6.06E+05 | 3.71E−04 | 1.63E+09 | 0.612 |
| 105-08 2F6 | 5.50E+05 | 3.59E−04 | 1.53E+09 | 0.653 |
| 105-08 2G10 | 3.02E+05 | 3.97E−04 | 7.58E+08 | 1.320 |
| 105-08 2G7 | 2.51E+05 | 3.58E−04 | 6.99E+08 | 1.430 |
| 105-20 1B3 | 4.05E+05 | 3.10E−04 | 1.31E+09 | 0.764 |
| 105-20 1H1 | 3.74E+05 | 3.20E−04 | 1.17E+09 | 0.857 |
| 105-20 1H7 | 5.00E+05 | 3.72E−04 | 1.34E+09 | 0.744 |
| 105-20 2A3 | 4.12E+05 | 3.12E−04 | 1.32E+09 | 0.759 |
| 105-20 2F12 | 2.54E+05 | 4.71E−04 | 5.41E+08 | 1.850 |
| 105-20 2G12 | 3.98E+05 | 2.62E−04 | 1.52E+09 | 0.658 |
| H4E D145A | 4.01E+05 | 2.86E−04 | 1.40E+09 | 0.714 |
| H4E E137A | 4.37E+05 | 2.61E−04 | 1.68E+09 | 0.597 |
| H4E E139A | 4.19E+05 | 2.68E−04 | 1.56E+09 | 0.64 |
| H4E N154A | 1.68E+05 | 1.42E−04 | 1.19E+09 | 0.843 |
| H4E Q143A | 3.42E+05 | 2.36E−04 | 1.45E+09 | 0.689 |
| H4E R170A | 3.23E+05 | 2.51E−04 | 1.29E+09 | 0.777 |
| H4E T138A | 3.52E+05 | 2.61E−04 | 1.35E+09 | 0.743 |
| H4E T141A | 4.05E+05 | 2.64E−04 | 1.54E+09 | 0.651 |
| H4EW | 6.51E+05 | 3.64E−04 | 1.79E+09 | 0.560 |

### Example 28

### ATRIMER™ Complexes Binding to IL-23R do not Recognize IL-12Rβ1 or IL-12Rβ2

A Biacore 3000 biosensor (GE Healthcare) was used to evaluate the interaction of human IL-12Rβ1/Fc or IL-12Rβ2/Fc with IL-23R binding ATRIMER™ complexes. Immobilization of an anti-human IgG Fc antibody (GE Healthcare) to the CM5 chip (GE Healthcare) was performed using standard amine coupling chemistry, and this modified surface was used to capture recombinant human IL-12Rβ1/Fc or IL-12Rβ2/Fc fusion protein (R&D Systems). A low-density receptor surface, less than 200 RU, was used for all of the analyses. ATRIMER™ complex dilutions (100 nM) were injected over the IL-12R surface at 30 μl/min. Data collection was 3 minutes for the association and 5 minutes for dissociation. The anti-human IgG surface was regenerated with a 30 s pulse of 3M magnesium chloride. All sensorgrams were double-referenced against an anti-human IgG Fc antibody surface as well as buffer injections. As shown in Table 24, ATRIMER™ complexes did not show any measurable binding to human IL-12Rβ1/Fc or IL-12Rβ2/Fc.

TABLE 24

| ATRIMER ™ | | |
|---|---|---|
| (100 nM) | Il12Rb1 | Il12Rb2 |
| 105-08-1A8 | negative | negative |
| H4E-E137A | negative | negative |
| 101-54-4B6 | negative | negative |
| 101-113-6C108 | negative | negative |
| 101-51-1A4 | negative | negative |
| 101-51-1A7 | negative | negative |
| 101-51-1A7F | negative | negative |
| 105-08-1A8 | negative | negative |

### Example 29

### Competitive Assays of Human IL-23 Binding to IL-23R in the Presence of IL-23R Binders Using Biacore

IL-23R binding ATRIMER™ polypeptide complexes were amine-coupled to CM5 chips (GE Healthcare) then IL-23R (IL-23R) was injected over the chip surface. Following binding stabilization, the ability of human IL-23 (eBioscience) to interact with IL-23R was monitored. Additional competition assays were done by pre-forming a complex between IL-23R and IL-23 or IL-23R and ATRIMER™ polypeptide complexes for 30 minutes at room temperature. The complex was then injected over the surface with the amine-coupled ATRIMER™ complexes. Remaining binding of IL-23R Atrimer, as shown in Table 25 for Atrimer A5F was determined and expressed as percent of binding in the absence of competitor (IL-23 or different Atrimer).

TABLE 25
A5F competes with binding of IL-23 to the IL-23R

| Analyte | Percent binding to A5F |
|---|---|
| rhIL23RFc | 100 |
| rhIL23RFc + rhIL23 | 19 |
| rhIL23RFc + A9E | 25 |

### Example 30

### Testing Activity of Selected Atrimer™ Polypeptide Complex in Cell Based Assay

Human peripheral blood mononuclear cells (PBMC) from healthy donors (AllCells) were stimulated at 1×10⁶ cells/mL with human recombinant IL-23 (1 ng/mL, eBioscience) and PHA (1 μg/mL, Sigma) in the presence of IL-23R ATRIMER™ polypeptide complexes or Ustekinumab in 10% FBS/Advanced RPMI media (Invitrogen). After 4 days in culture, cell supernatants were collected and assayed by ELISA using IL-17 Quantikine kits (R&D Systems). In parallel cultures, PBMC were treated with human recombinant IL-12 (1 ng/mL, R&D Systems) in the presence of IL-23R ATRIMER™ polypeptide complexes or Ustekinumab for 4 days. Cell supernatants were assayed for IFNγ and IL-17 by Luminex (Procarta, Panomics) and analyzed on the Bioplex system (BioRad). All treatments were performed in triplicate, and the mean and standard error were plotted using GraphPad Prism software. As shown in FIGS. 12, 13 and 14, IL-23 ATRIMER™ polypeptide complexes blocked IL-23-induced IL-17 production, but did not inhibit IL-12-induced IFNγ production. As expected, Ustekinumab inhibited both IL-23 and IL-12 responses.

Table 26 shows the results for affinity-matured ATRIMER™ polypeptide complexes tested in the PBMC assay. The ability of the ATRIMER™ polypeptide complexes to block IL-23-induced IL-17, IL-17F, and IL-22 production was measured for ATRIMER™ polypeptide complexes as indicated. The results are shown as a ratio with the numerator being the IC50 for the ATRIMER™ polypeptide complexes compared to the IC50 for ustekinumab. Results of more than one assay are shown for some ATRIMER™ polypeptide complexes.

TABLE 26
Production levels of the indicated cytokines in the presence of each
ATRIMER ™ polypeptide complex compared to ustekinumab
in the same experiment.
(Atrimer/Ustekinumab)

| ATRIMER ™ | | | |
|---|---|---|---|
| complex | IL17 | IL-17F | IL22 |
| 101-113-6C108 | 0.013/1.03 | 0.41/0.77 | |
| 105-08 1A8 | 0.14/0.16 | 0.42/0.1 | |
| 101-51-1A4 | 0.2/1.03 | 4.9/1.05 | 0.27/0.09 |
| | 0.12/0.47 | 0.09/0.25 | |
| 101-54-4B6 | 0.1/0.47 | 0.18/0.25 | 0.12/0.09 |
| | 8.8/0.56 | 5.2/0.55 | |
| | 0.15/0.16 | 0.11/0.1 | |
| H4E E137A | 1.4/0.73 | 2.1/0.34 | |
| | | 16/0.55 | |
| 101-51-1A7 | 1.8/0.58 | 4.4/0.44 | |
| 101-54-4B3 | 3.6/0.16 | 0.16/0.1 | |
| 105-08 2C10 | 3.1/0.47 | 5.2/0.25 | 1.8/0.09 |
| 101-54-4B10 | 4.4/0.93 | 6.6/2.3 | |
| 101-80-5E8 | 7.9/1.03 | 12.9/0.77 | |
| 105-20 1H7 | 16/0.33 | 4.2/0.43 | |
| H4E T138A | 8.8/0.73 | 13/0.34 | |
| 056-53 H4E | 17/0.73 | 45/0.34 | |
| 101-51-1A5 | 34/0.58 | 18/0.44 | |
| 105-08 1B7 | 19/0.93 | 225/2.3 | |
| 105-08 1D3 | 109/0.58 | 31/0.44 | |
| 105-20 2G12 | 158/0.93 | 601/2.3 | |
| 105-08 1A3 | 233/3.0 | 201/3.3 | |

### Example 31

### NKL Agonist Assay

To show the lack of agonist activity of IL-23R ATRIMER™ polypeptide complexes on IL-23R, STAT-3 phosphorylation upon binding of selected IL-23R ATRIMER™ complexes to the natural killer cell line NKL expressing the heterodimeric IL-23 receptor was determined. ATRIMER™ complexes at a concentration of 150 ug/mL or IL-23 at 50 ng/mL as positive control were incubated at 37° C. with 140,000 NKL cells/well in a 96 well plate. After 10 min, cells were centrifuged at 1200 rpm for 5 min, and washed with PBS twice. Then, cells were lysed and treated according to the protocol provided in the Stat3 phosphorylation kit that was obtained from Cell signaling technology (PATH SCAN® Phospho Stat3 Sandwich ELISA kit, Cat #7300, Cell Signalling Technlogy, Inc., Danvers, Mass.). Stat-3 phosphorylation was measured by absorbance at 450 nM using a Molecular Devices ELISA reader. As shown in FIG. 15 exemplary for complexes of H4E and H4EP1E9, no activation IL-23R receptor by the complexes was observed, while IL-23 resulted in STAT-3 phosphorylation as expected. Similar results were obtained for all other atrimers tested such as 101-51-1A4, 101-51-1A7, 105-08-1A8, 101-54-4B6, H4E E137A, 101-113-6C108 and 101-54-4B10 as summarized in FIGS. 16A and 16B

### Example 32

### Panning of Mouse 1-4 Library on Mouse IL-23R and Identification of a Mouse IL-23R-Specific CTLD Binder

Panning & Screening of Mouse Library 1-4

Phage generated from mouse library 1-4 were panned on recombinant mouse IL-23R/Fc chimera (R&D Systems). Screening of these binding panels using an ELISA plate assay after three rounds of panning identified a receptor-specific binder.

To generate phage for panning, the master library DNA was transformed by electroporation into bacterial strain ER2738 (Lucigen or NEB). Cells were allowed to recover for one hour with shaking at 37° C. in SOC (Super-Optimal broth with Catabolite repression) medium prior to increasing the volume 10-fold by adding super broth (SB) to a final concentration of 20% glucose and 20 μg/mL carbenicillin. After shaking at 37° C. for one hour, the carbenicillin concentration was increased to 50 μg/mL for another hour, after which 400 mL of SB with 2% glucose and 50 μg/mL carbenicillin were added, along with helper phage M13K07 to a final concentration of 5×10⁹ pfu/mL. Incubation was continued at 37° C. without shaking for 30 minutes, and then with shaking at 100-150 rpm for another 30 min. Cells were centrifuged at 3200 g at 4° C. for 20 minutes, then resuspended in 500 mL SB medium containing 50 μg/mL carbenicillin and 50 μg/mL kanamycin. Cells were grown overnight at room temperature (RT) with shaking at 150 rpm. Phage were isolated by pelleting the bacterial cells by centrifugation at 15,000 g and 4° C. for 20 min. The supernatant was incubated with one-fourth volume (usually 250 mL of supernatant/bottle+62.5 mL PEG solution) of 20% PEG/2.5 M NaCl on ice for 30 min. The phage was pelleted by centrifugation at 15,000 g and 4° C. for 20 min. The phage pellet was resuspended in Buffer D, containing 0.05% boiled cassein, 0.025% Tween-20, and protease inhibitors. Material was filter-sterilized using Whatman Puradisc 25 mm diameter, 0.2 μm pore size filters.

Phage generated from mouse library 1-4 were panned on recombinant mouse IL-23R/Fc chimera (R&D Systems cat #1686-MR) using a plate format. Six wells of a 96-well Immulon HB2 ELISA plate were coated with 250-1000 ng/well of carrier-free mouse IL-23R/Fc in Dulbecco's PBS. Material was incubated on the plate overnight, after which wells were washed three times with PBS and blocking buffer ((Buffer C, containing 0.05% boiled casseing and 1% Tween-20) was added. Wells were incubated for at least 1 hour at 37° C. Additional wells were also treated with blocking buffer at the same time for later absorption of phage binding to blocking buffer.

Three dilutions of the phage preparation were used: undiluted, 1:10, and 1:100 in buffer D plus protease inhibitors. In the 3 round of panning, recombinant human IgG1 Fc was added to each of the dilutions to a final concentration of 10 μg/mL. Blocking buffer was removed from the “Block Only” (preabsorption to block) wells and the different phage mixtures were incubated in these wells for another hour at 37° C. Aliquots (50 μL) of each phage mixture were transferred to a washed and blocked target well and allowed to incubate for 2 h at 37° C. For the first round of panning, bound phage were washed once with Buffer D, and were eluted using glycine buffer, pH 2.2, containing 1 mg/mL BSA. After neutralization with 2 M Tris base (pH 11.5) the eluted phage were incubated for 15 minutes at room temperature with two to four milliliters of ER2738 cells (Lucigen or NEB) at an optical density of approximately 0.9 measured at 600 nm (OD₆₀₀) in yeast extract-tryptone (YT) medium. Phage were prepared from this infection using the protocol above, but scaled down by about 20% (volume). Phage prepared from eluted phage were subjected to additional rounds of panning. At each round, titers of input and output phage were determined by plating on agar with appropriate antibiotics, and colonies from these plates were used later for screening for binders by ELISA.

Additional rounds of panning were performed as described above, except that in the second round of panning, washes were increased to 5×, and in subsequent rounds, washes were increased to 10×. Three to six rounds of panning were performed. For the final round of panning, phage were not produced after infection; rather, infected bacteria were grown overnight and a maxiprep (Qiagen kit) was prepared from the DNA. Glycerol stocks (15%) of input phage were stored frozen (at −80° C.) from each round.

For ELISA screening, colonies from later rounds of panning were grown in YT medium with 2% glucose and antibiotics overnight, and an aliquot of each was then used to start fresh cultures that were grown to an OD₆₀₀ of 0.5. Helper phage were added to 5×10⁹ pfu/mL and allowed to infect for 30 min at 37° C., followed by growth at 37° C. with agitation. Bacteria were centrifuged and resuspended in YT medium with carbenicillin and kanamycin and grown overnight for phage production. Bacteria were then pelleted and the medium was removed and mixed with one-fifth volume (1:5 milk mixture:supernatant) of 6×PBS, 18% milk. ELISA plates were prepared by incubating overnight at 4° C. with 50-100 μL of PBS containing 75-100 ng/well of recombinant mouse IL-23R/Fc. A duplicate plate coated with human IgG Fc (R&D Systems) was used as a control. Plates were washed 3 times with PBS, blocked for 1 h at 37° C. with 3% milk in 1×PBS, and incubated for 1 hour with 100 uL/well of each milk-treated phage mixture. Plates were washed once with PBS/0.05% Tween 20 and twice with PBS, incubated for one hour with an HRP-conjugated anti-M13 antibody (GE Healthcare), washed three times each with PBS/Tween and PBS, and incubated with TMB substrate (VWR). Sulfuric acid was added to stop the color reaction and absorbance was read at 450 nm to identify positive binders.

A phage-displayed mouse TN CTLD that bound well to mouse IL-23R was identified from the third round of panning. The sequence from the randomized regions of Loops 1 and 4 from this binder is given in Table 27.

TABLE 27

| | | SEQ | | SEQ |
|---|---|---|---|---|
| | | ID | | ID |
| Clone name | Loop1 | NO | Loop4 | NO |
| 105-106-6F1 | PGPGTRW | 576 | RSKSG | 577 |

The above examples do not limit the scope of variation that can be generated in these libraries. Other libraries can be generated in which varying numbers of random or more targeted amino acids are used to replace existing amino acids, and different combinations of loops can be utilized. In addition, other mutations and methods of generating mutations, such as random PCR mutagenesis, can be utilized to provide diverse libraries that can be subjected to panning.

Although various specific embodiments of the present invention have been described herein, it is to be understood that the invention is not limited to those precise embodiments and that various changes or modifications can be affected therein by one skilled in the art without departing from the scope and spirit of the invention.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention.

The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

The disclosures of all references and publications cited herein are expressly incorporated by reference in their entireties to the same extent as if each were incorporated by reference individually.

### REFERENCES

Aspberg, A., Miura, R., Bourdoulous, S., Shimonaka, M., Heinegard, D., Schachner, M., Ruoslahti, E., and Yamaguchi, Y. (1997). “The C-type lectin domains of lecticans, a family of aggregating chondroitin sulfate proteoglycans, bind tenascin-R by protein-protein interactions independent of carbohydrate moiety”. Proc. Natl. Acad. Sci. (USA) 94: 10116-10121
Bass, S., Greene, R., and Wells, J. A. (1990). “Hormone phage: an enrichment method for variant proteins with altered binding properties”. Proteins 8: 309-314
Benhar, I., Azriel, R., Nahary, L., Shaky, S., Berdichevsky, Y., Tamarkin, A., and Wels, W. (2000). “Highly efficient selection of phage antibodies mediated by display of antigen as Lpp-OmpA′ fusions on live bacteria”. J. Mol. Biol. 301: 893-904
Berglund, L. and Petersen, T. E. (1992). “The gene structure of tetranectin, a plasminogen binding protein”. FEBS Letters 309: 15-19
Bertrand, J. A., Pignol, D., Bernard, J-P., Verdier, J-M., Dagorn, J-C., and Fontecilla-Camps, J. C. (1996). “Crystal structure of human lithostathine, the pancreatic inhibitor of stone formation”. EMBO J. 15: 2678-2684
Bettler, B., Texido, G., Raggini, S., Ruegg, D., and Hofstetter, H. (1992). “Immunoglobulin E-binding site in Fc epsilon receptor (Fc epsilon R11/CD23) identified by homolog-scanning mutagenesis”. J. Biol. Chem. 267: 185-191
Blanck, O., Iobst, S. T., Gabel, C., and Drickamer, K. (1996). “Introduction of selectin-like binding specificity into a homologous mannose-binding protein”. J. Biol. Chem. 271: 7289-7292
Boder, E. T. and Wittrup, K. D. (1997). “Yeast surface display for screening combinatorial polypeptide libraries”. Nature Biotech. 15: 553-557
Burrows L, Iobst S T, Drickamer K. (1997) “Selective binding of N-acetylglucosamine to the chicken hepatic lectin”. Bio-chem J. 324:673-680
Chiba, H., Sano, H., Saitoh, M., Sohma, H., Voelker, D. R., Akino, T., and Kuroki, Y. (1999). “Introduction of mannose binding protein-type phosphatidylinositol recognition into pulmonary surfactant protein A”. Biochemistry 38: 7321-7331
Christensen, J. H., Hansen, P. K., Lillelund, O., and Thogersen, H. C. (1991). “Sequence-specific binding of the N-terminal three-finger fragment of Xenopus transcription factor IIIA to the internal control region of a 5S RNA gene”. FEBS Letters 281: 181-184
Cyr, J. L. and Hudspeth, A. J. (2000). “A library of bacteriophage-displayed antibody fragments directed against proteins of the inner ear”. Proc. Natl. Acad. Sci. (USA) 97: 2276-2281
Drickamer, K. (1992). “Engineering galactose-binding activity into a C-type mannose-binding protein”. Nature 360: 183-186
Drickamer, K. and Taylor, M. E. (1993). “Biology of animal lectins”. Annu Rev. Cell Biol. 9: 237-264
Drickamer, K. (1999). “C-type lectin-like domains”. Curr. Opinion Struc. Biol. 9: 585-590
Dunn, I. S. (1996). “Phage display of proteins”. Curr. Opinion Biotech. 7: 547-553
Erbe, D. V., Lasky, L. A., and Presta, L. G. “Selectin variants”. U.S. Pat. No. 5,593,882
Ernst, W. J., Spenger, A., Toellner, L., Katinger, H., Grabherr, R. M. (2000). “Expanding baculovirus surface display. Modification of the native coat protein gp64 of Autographa californica NPV”. Eur. J. Biochem. 267: 4033-4039
Ewart, K. V., Li, Z., Yang, D. S. C., Fletcher, G. L., and Hew, C. L. (1998). “The ice-binding site of Atlantic herring antifreeze protein corresponds to the carbohydrate-binding site of C-type lectins”. Biochemistry 37: 4080-4085
Feinberg, H., Park-Snyder, S., Kolatkar, A. R., Heise, C. T., Taylor, M. E., and Weis, W. I. (2000). “Structure of a C-type carbohydrate recognition domain from the macrophage mannose receptor”. J. Biol. Chem. 275: 21539-21548
Fujii, I., Fukuyama, S., Iwabuchi, Y., and Tanimura, R. (1998). “Evolving catalytic antibodies in a phage-displayed combinatorial library”. Nature Biotech. 16: 463-467
Gates, C. M., Stemmer, W. P. C., Kaptein, R., and Schatz, P. J. (1996). “Affinity selective isolation of ligands from peptide libraries through display on a lac repressor “headpiece dimer”. J. Mol. Biol. 255: 373-386
Graversen, J. H., Lorentsen, R. H., Jacobsen, C., Moestrup, S. K., Sigurskjold, B. W., Thogersen, H. C., and Etzerodt, M. (1998). “The plasminogen binding site of the C-type lectin tetranectin is located in the carbohydrate recognition domain, and binding is sensitive to both calcium and lysine”. J. Biol. Chem. 273:29241-29246
Graversen, J. H., Jacobsen, C., Sigurskjold, B. W., Lorentsen, R. H., Moestrup, S. K., Thogersen, H. C., and Etzerodt, M. (2000). “Mutational Analysis of Affinity and Selectivity of Kringle-Tetranectin Interaction. Grafting novel kringle affinity onto the tetranectin lectin scaffold”. J. Biol. Chem. 275: 37390-37396
Griffiths, A. D. and Duncan, A. R. (1998). “Strategies for selection of antibodies by phage display”. Curr. Opinion Biotech. 9: 102-108
Holtet, T. L., Graversen, J. H., Clemmensen, I., Thogersen, H. C., and Etzerodt, M. (1997). “Tetranectin, a trimeric plasminogen-binding C-type lectin”. Prot. Sci. 6: 1511-1515
Honma, T., Kuroki, Y., Tzunezawa, W., Ogasawara, Y., Sohma, H., Voelker, D. R., and Akino, T. (1997). “The mannose-binding protein A region of glutamic acid185-alanine221 can functionally replace the surfactant protein A region of glutamic acid195-phenylalanine228 without loss of interaction with lipids and alveolar type II cells”. Biochemistry 36: 7176-7184
Huang, W., Zhang, Z., and Palzkill, T. (2000). “Design of potent beta-lactamase inhibitors by phage display of beta-lactamase inhibitory protein”. J. Biol. Chem. 275: 14964-14968
Hufton, S. E., van Neer, N., van den Beuken, T., Desmet, J., Sablon, E., and Hoogenboom, H. R. (2000). “Development and application of cytotoxic T lymphocyte-associated antigen 4 as a protein scaffold for the generation of novel binding ligands”. FEBS Letters 475: 225-231
Hakansson, K., Lim, N. K., Hoppe, H-J., and Reid, K. B. M. (1999). “Crystal structure of the trimeric alpha-helical coiled-coil and the three lectin domains of human lung surfactant protein D”. Structure Folding and Design 7: 255-264
Iobst, S. T., Wormald, M. R., Weis, W. I., Dwek, R. A., and Drickamer, K. (1994). “Binding of sugar ligands to Ca(2+)-dependent animal lectins. I. Analysis of mannose binding by site-directed mutagenesis and NMR”. J. Biol. Chem. 269: 15505-15511
Iobst, S. T. and Drickamer, K. (1994). “Binding of sugar ligands to Ca(2+)-dependent animal lectins. II. Generation of high-affinity galactose binding by site-directed mutagenesis”. J. Biol. Chem. 269: 15512-15519
Iobst, S. T. and Drickamer, K. (1996). “Selective sugar binding to the carbohydrate recognition domains of the rat hepatic and macrophage asialoglycoprotein receptors”. J. Biol. Chem. 271: 6686-6693
Jaquinod, M., Holtet, T. L., Etzerodt, M., Clemmensen, I., Thogersen, H. C., and Roepstorff, P. (1999). “Mass Spectrometric Characterisation of Post-Translational Modification and Genetic Variation in Human Tetranectin”. Biol. Chem. 380: 1307-1314
Kastrup, J. S., Nielsen, B. B., Rasmussen, H., Holtet, T. L., Graversen, J. H., Etzerodt, M., Thogersen, H. C., and Larsen, I. K. (1998). “Structure of the C-type lectin carbohydrate recognition domain of human tetranectin”. Acta. Cryst. D 54: 757-766
Kogan, T. P., Revelle, B. M., Tapp, S., Scott, D., and Beck, P. J. (1995). “A single amino acid residue can determine the ligand specificity of E-selectin”. J. Biol. Chem. 270: 14047-14055
Kolatkar, A. R., Leung, A. K., Isecke, R., Brossmer, R., Drickamer, K., and Weis, W. I. (1998). “Mechanism of N-acetylgalactosamine binding to a C-type animal lectin carbohydrate-recognition domain”. J. Biol. Chem. 273: 19502-19508
Lorentsen, R. H., Graversen, J. H., Caterer, N. R., Thogersen, H. C., and Etzerodt, M. (2000). “The heparin-binding site in tetranectin is located in the N-terminal region and binding does not involve the carbohydrate recognition domain”. Biochem. J. 347: 83-87
Marks, J. D., Hoogenboom, H. R., Griffiths, A. D., and Winter, G. (1992). “Molecular evolution of proteins on filamentous phage. Mimicking the strategy of the immune system”. J. Biol. Chem. 267: 16007-16010
Mann K, Weiss I M, Andre S, Gabius H J, Fritz M. (2000). “The amino-acid sequence of the abalone (Haliotis laevigata) nacre protein perlucin. Detection of a functional C-type lectin domain with galactose/mannose specificity”. Eur. J. Biochem. 267: 5257-5264
McCafferty, J., Jackson, R. H., and Chiswell, D. J. (1991). “Phage-enzymes: expression and affinity chromatography of functional alkaline phosphatase on the surface of bacterio-phage”. Prot. Eng. 4: 955-961
McCormack, F. X., Kuroki, Y., Stewart, J. J., Mason, R. J., and Voelker, D. R. (1994). “Surfactant protein A amino acids Glu195 and Arg197 are essential for receptor binding, phospholipid aggregation, regulation of secretion, and the facilitated uptake of phospholipid by type II cells”. J. Biol. Chem. 269: 29801-29807
McCormack, F. X., Festa, A. L., Andrews, R. P., Linke, M., and Walzer, P. D. (1997). “The carbohydrate recognition domain of surfactant protein A mediates binding to the major surface glycoprotein of Pneumocystis carinii”. Biochemistry 36: 8092-8099
Meier, M., Bider, M. D., Malashkevich, V. N., Spiess, M., and Burkhard, P. (2000). “Crystal structure of the carbohydrate recognition domain of the Hi subunit of the asialoglycoprotein receptor”. J. Mol. Biol. 300: 857-865
Mikawa, Y. G., Maruyama, I. N., and Brenner, S. (1996). “Surface display of proteins on bacteriophage lambda heads”. J. Mol. Biol. 262: 21-30
Mio H, Kagami N, Yokokawa S, Kawai H, Nakagawa S, Takeuchi K, Sekine S, Hiraoka A. (1998). “Isolation and characterization of a cDNA for human mouse, and rat full-length stem cell growth factor, a new member of C-type lectin superfamily”. Biochem. Biophys. Res. Commun. 249: 124-130
Mizuno, H., Fujimoto, Z., Koizumi, M., Kano, H., Atoda, H., and Morita, T. (1997). “Structure of coagulation factors IX/X-binding protein, a heterodimer of C-type lectin domains”. Nat. Struc. Biol. 4: 438-441 [0287] Ng, K. K., Park-Snyder, S., and Weis, W. I. (1998a). “Ca.sup.2+-dependent structural changes in C-type mannose-binding proteins”. Biochemistry 37: 17965-17976
Ng, K. K. and Weis, W. I. (1998b). “Coupling of prolyl peptide bond isomerization and Ca2+ binding in a C-type mannose-binding protein”. Biochemistry 37: 17977-17989
Nielsen, B. B., Kastrup, J. S., Rasmussen, H., Holtet, T. L., Graversen, J. H., Etzerodt, M., Thogersen, H. C., and Larsen, I. K. (1997). “Crystal structure of tetranectin, a trimeric plasminogen-binding protein with an alpha-helical coiled coil”. FEBS Letters 412: 388-396
Nissim A., Hoogenboom, H. R., Tomlinson, I. M., Flynn, G., Midgley, C., Lane, D., and Winter, G. (1994). “Antibody fragments from a ‘single pot’ phage display library as immunochemical reagents”. EMBO J. 13: 692-698
Ogasawara, Y. and Voelker, D. R. (1995). “Altered carbohydrate recognition specificity engineered into surfactant protein D reveals different binding mechanisms for phosphatidylinositol and glucosylceramide”. J. Biol. Chem. 270: 14725-14732
Ohtani, K., Suzuki, Y., Eda, S., Takao, K., Kase, T., Yamazaki, H., Shimada, T., Keshi, H., Sakai, Y., Fukuoh, A., Sakamoto, T., and Wakamiya, N. (1999). “Molecular cloning of a novel human collectin from liver (CL-L1)”. J. Biol. Chem. 274: 13681-13689
Pattanajitvilai, S., Kuroki, Y., Tsunezawa, W., McCormack, F. X., and Voelker, D. R. (1998). “Mutational analysis of Arg197 of rat surfactant protein A. His197 creates specific lipid uptake defects”. J. Biol. Chem. 273: 5702-5707
Poget, S. F., Legge, G. B., Proctor, M. R., Butler, P. J., Bycroft, M., and Williams, R. L. (1999). “The structure of a tunicate C-type lectin from Polyandrocarpa misakiensis complexed with D-galactose”. J. Mol. Biol. 290: 867-879
Revelle, B. M., Scott, D., Kogan, T. P., Zheng, J., and Beck, P. J. (1996). “Structure-function analysis of P-selectinsialyl LewisX binding interactions. Mutagenic alteration of ligand binding specificity”. J. Biol. Chem. 271: 4289-4297
Sano, H., Kuroki, Y., Honma, T., Ogasawara, Y., Sohma, H., Voelker, D. R., and Akino, T. (1998). “Analysis of chimeric proteins identifies the regions in the carbohydrate recognition domains of rat lung collections that are essential for interactions with phospholipids, glycolipids, and alveolar type II cells”. J. Biol. Chem. 273: 4783-4789
Schaffitzel, C., Hanes, J., Jermutus, L., and Plucktun, A. (1999). “Ribosome display: an in vitro method for selection and evolution of antibodies from libraries”. J. Immunol. Methods 231: 119-135
Sheriff, S., Chang, C. Y., and Ezekowitz, R. A. (1994). “Human mannose-binding protein carbohydrate recognition domain trimerizes through a triple alpha-helical coiled-coil”. Nat. Struc. Biol. 1: 789-794
Sorensen, C. B., Berglund, L., and Petersen, T. E. (1995). “Cloning of a cDNA encoding murine tetranectin”. Gene 152: 243-245
Torgersen, D., Mullin, N. P., and Drickamer, K. (1998). “Mechanism of ligand binding to E- and P-selectin analyzed using selectin/mannose-binding protein chimeras”. J. Biol. Chem. 273: 6254-6261
Tormo, J., Natarajan, K., Margulies, D. H., and Mariuzza, R. A. (1999). “Crystal structure of a lectin-like natural killer cell receptor bound to its MHC class I ligand”. Nature 402: 623-631
Tsunezawa, W., Sano, H., Sohma, H., McCormack, F. X., Voelker, D. R., and Kuroki, Y. (1998). “Site-directed mutagenesis of surfactant protein A reveals dissociation of lipid aggregation and lipid uptake by alveolar type II cells”. Biochim. Biophys. Acta 1387: 433-446
Weis, W. I., Kahn, R., Fourme, R., Drickamer, K., and Hendrickson, W. A. (1991). “Structure of the calcium-dependent lectin domain from a rat mannose-binding protein determined by MAD phasing”. Science 254: 1608-1615
Weis, W. I., and Drickamer, K. (1996). “Structural basis of lectin-carbohydrate recognition”. Annu Rev. Biochem. 65: 441-473
Whitehorn, E. A., Tate, E., Yanofsky, S. D., Kochersperger, L., Davis A., Mortensen, R. B., Yonkovic, S., Bell, K., Dower, W. J., and Barrett, R. W. (1995). “A generic method for expression and use of “tagged” soluble versions of cell surface receptors”. Bio/Technology 13: 1215-1219
Wragg, S, and Drickamer, K. (1999). “Identification of amino acid residues that determine pH dependence of ligand binding to the asialoglycoprotein receptor during endocytosis”. J. Biol. Chem. 274: 35400-35406
Zhang, H., Robison, B., Thorgaard, G. H., and Ristow, S. S. (2000). “Cloning, mapping and genomic organization of a fish C-type lectin gene from homozygous clones of rainbow trout (Oncorhynchos Mykiss)”. Biochim. et Biophys. Acta 1494: 14-22
Agnew, Chem. Intl. Ed. Engl., 33: 183-186 (1994)
Ashkenazi, et al. J Clin Invest.; 104(2):155-62 (July 1999).
Chemotherapy Service Ed., M. C. Perry, Williams & Wilkins, Baltimore, Md. (1992)
Ausubel et al., Current Protocols in Molecular Biology (eds., Green Publishers Inc. and Wiley and Sons 1994
Degli-Esposti et al., Immunity, 7(6):813-820 (December 1997)
Degli-Esposti et al., J. Exp. Med., 186(7):1165-1170 (Oct. 6, 1997)
Janeway, Nature, 341(6242): 482-3 (Oct. 12, 1989)
Jin et al, Cancer Res., 15; 64(14):4900-5 (July 2004).
Langer et al., J. Biomed. Mater. Res., 15: 167-277 (1981)
Langer, Chem. Tech., 12: 98-105 (1982)
Marsters et al., Curr. Biol., 7:1003-1006 (1997)
McFarlane et al., J. Biol. Chem., 272:25417-25420 (1997)
Mongkolsapaya et al., J. Immunol., 160:3-6 (1998)
Mordenti et al., Pharmaceut. Res., 8:1351 (1991)
Neame, et al., Protein Sci., 1(1):161-8 (1992)
Neame, P. J. and Boynton, R. E., Protein Soc. Symposium, (Meeting date 1995; 9th Meeting: Tech. Prot. Chem. VII). Proceedings pp. 401-407 (Ed., Marshak, D. R.; Publisher: Academic, San Diego, Calif.) (1996).
Offner et al., Science, 251: 430-432 (1991)
Pan et al., FEBS Letters, 424:41-45 (1998)
Pan et al., Science, 276:111-113 (1997)
Pan et al., Science, 277:815-818 (1997)
Remington's Pharmaceutical Sciences, 16th edition, Osol, A. ed. (1980)
S. G. Hymowitz, et. al., Mol. Cell. 1999 October; 4(4):563-71)
Sambrook, et al. Molecular Cloning: A Laboratory Manual. Cold Spring Harbor Laboratory Press, Cold Spring Harbor, N.Y. (1989)
Schneider et al., FEBS Letters, 416:329-334 (1997)
Screaton et al., Curr. Biol., 7:693-696 (1997)
Sheridan et al., Science, 277:818-821 (1997)
Sidman et al., Biopolymers, 22: 547-556 (1983)
Cha et. al., J Biol. Chem., 275(40):31171-7 (Oct. 6, 2000).
Murakami et al., The Molecular Basis of Cancer, Mendelsohn and Israel, eds., Chapter 1, entitled “Cell cycle regulation, oncogenes, and antineoplastic drugs” by (W B Saunders: Philadelphia, pg. 13 (1995).
Walczak et al., EMBO J., 16:5386-5387 (1997)
Wu et al., Nature Genetics, 17:141-143 (1997)

1. A combinatorial polypeptide library comprising polypeptide members that comprise a C-type lectin domain (CTLD) having a randomized loop region, wherein the CTLD loop region comprises loop segment A (LSA) containing Loops 1-4 and loop segment B (LSB) containing Loop 5 and is randomized according to one of the following Schemes:
(a) amino acid modifications in at least one of the four loops in loop segment A (LSA) of the CTLD, wherein the amino acid modifications comprise an insertion of at least one amino acid in Loop 1 and random substitution of at least five amino acids within Loop 1;
(b) amino acid modifications in at least one of the four loops in loop segment A (LSA) of the CTLD, wherein the amino acid modifications comprise random substitution of at least five amino acids within Loop 1 and random substitution of at least three amino acids within Loop 2;
(c) amino acid modifications in at least one of the four loops in the loop segment A (LSA) of the CTLD, wherein the amino acid modifications comprise random substitution of at least seven amino acids within Loop 1 and at least one amino acid insertion in Loop 4;
(d) amino acid modifications in at least one of the four loops in the loop segment A (LSA) of the CTLD, wherein the amino acid modifications comprise at least one amino acid insertion in Loop 3 and random substitution of at least three amino acids within Loop 3;
(e) amino acid modifications in at least one of the four loops in the loop segment A (LSA) of the CTLD, wherein the amino acid modifications comprise a modification that combines two loops into a single loop, wherein the two combined loops are Loop 3 and Loop 4;
(f) amino acid modifications in at least one of the four loops in the loop segment A (LSA) of the CTLD, wherein the amino acid modifications comprise at least one amino acid insertion in Loop 4 and random substitution of at least three amino acids within Loop 4;
(g) amino acid modifications in at least one of the four loops in the loop segment A (LSA) of the CTLD and in loop segment B (LSB), wherein the amino acid modifications comprise random substitution of at least five amino acid residues in Loop 3 and random substitution of at least three amino acids within Loop 5;
(h) amino acid modifications in at least one of the four loops in the loop segment A (LSA) of the CTLD, wherein the amino acid modifications comprise random substitution of at least one amino acid and insertion of at least six amino acids in Loop 3;
(i) amino acid modifications in at least one of the four loops in the loop segment A (LSA) of the CTLD, wherein the amino acid modifications comprise a mixture of (1) random substitution of at least six amino acids in Loop 3 and (2) random substitution of at least six amino acids and at least one amino acid insertion in Loop 3; and
(j) amino acid modifications in at least one of the four loops in the loop segment A (LSA) of the CTLD, wherein the amino acid modifications comprise at least four or more amino acid insertions in at least one of the four loops in the loop segment A (LSA) or loop 5 in loop segment B (LSB) of the CTLD.

2. The library of claim 1, wherein the CTLD comprises the following secondary structure:
(a) five β-strands and two α-helices sequentially appearing in the order β1, α1, α2, β2, β3, β4, and β5, the β-strands being arranged in two anti-parallel β-sheets, one composed of β1 and β5, the other composed of β2, β3 and β4;
(b) at least two disulfide bridges, one connecting α1 and β5 and one connecting β3 and the polypeptide segment connecting β4 and β5; and
(c) a loop segment A (LSA) and a loop segment B (LSB), wherein LSA connects β2 and β3, and LSB connects β3 and β4.

3. The library of claim 1, further comprising random substitution of the amino acid located adjacent to the C-terminal end of Loop 2 in the C-terminal direction.

4. The combinatorial library of claim 1, wherein the CTLD is from human tetranectin and further comprises random substitution of Arginine-130.

5. The combinatorial library of claim 1, wherein the CTLD is from human or mouse tetranectin and further comprises a substitution of Lysine-148 to Alanine.

6. The combinatorial library of claim 4 having the randomized CTLD of Scheme (a), wherein the amino acid modifications comprise two amino acid insertions in Loop 1, random substitution of at least five amino acids within Loop 1, and a substitution of Lysine-148 to Alanine.

7. The combinatorial library of claim 1 having the randomized CTLD of Scheme (c), wherein the amino acid modifications further comprise random substitution of at least two amino acids within Loop 4.

8. The combinatorial library of claim 7, wherein the amino acid modifications comprise random substitution of at least seven amino acids within Loop 1, at least three amino acid insertions in Loop 4, and random substitution of at least two amino acids within Loop 4.

9. The combinatorial library of claim 1 having the randomized CTLD of Scheme (d), wherein the amino acid modifications further comprise at least one amino acid insertion in Loop 4.

10. The combinatorial library of claim 9, wherein the amino acid modifications further comprise random substitution of at least three amino acids within Loop 4.

11. The combinatorial library of claim 10, wherein the amino acid modifications comprise three amino acid insertions in Loop 3.

12. The combinatorial library of claim 11, wherein the amino acid modifications comprise three amino acid insertions in Loop 4.

13. The combinatorial library of claim 1 having the randomized CTLD of Scheme (e), wherein the amino acid modifications comprise random substitution of at least six amino acids in Loop 3 and random substitution of at least four amino acids in Loop 4.

14. The combinatorial library of claim 13, wherein the CTLD is human or mouse tetranectin and wherein the amino acid modifications further comprise random substitution of Proline-144.

15. The combinatorial library of claim 14, wherein the combined Loop 3 and Loop 4 amino acid sequence comprises NWEXXXXXXX XGGXXXN (SEQ ID NO: 578), wherein X is any amino acid and wherein the amino acid sequence of SEQ ID NO: 578 forms a single Loop region.

16. The combinatorial library of claim 1 having the randomized CTLD of Scheme (f), wherein the amino acid modifications comprise four amino acid insertions in Loop 4 and random substitution of at least three amino acids within Loop 4.

17. The combinatorial library of claim 1 having the randomized CTLD of Scheme (g), further comprising one or more amino acid modifications in the Loop 4 region that modulates plasminogen-binding affinity of the CTLD.

18. The combinatorial library of claim 17, wherein the CTLD is from human or mouse tetranectin and the modification to Loop 4 comprises substitution of Lysine 148 to Alanine.

19. The combinatorial library of claim 1 having the randomized CTLD of Scheme (h), wherein the CTLD is from human or mouse tetranectin and wherein the amino acid modifications comprise random substitution of Isoleucine 140.

20. The combinatorial library of claim 19, further comprising one or more amino acid modifications in the Loop 4 region that modulates plasminogen-binding affinity of the CTLD.

21. The combinatorial library of claim 20, wherein the modification to Loop 4 comprises substitution of Lysine 148 to Alanine.

22. The combinatorial library of claim 1 having the randomized CTLD of Scheme (i), wherein the amino acid modifications comprise amino acid modifications in at least one of the four loops in the loop segment A (LSA) of the CTLD, wherein the amino acid modifications comprise a mixture of (1) random substitution of at least six amino acids in Loop 3; (2) random substitution of at least six amino acids and at least one amino acid insertion in Loop 3; and (3) random substitution of at least six amino acids and at least two amino acid insertions in Loop 3;

23. The combinatorial polypeptide library of claim 2, wherein the CTLD comprises one or more amino acid modifications in any combination of two, three, four, or five of the loops in loop segment A (LSA) and loop segment B (LSB).

24. The combinatorial library of claim 1, wherein the amino acid modifications comprise modifications to CTLD amino acids outside of the LSA and LSB.

25. The combinatorial library of claim 1 wherein the CTLD is that of human tetranectin.

26. The combinatorial library of claim 1 wherein the CTLD is that of murine tetranectin.

27. The combinatorial library of claim 1, wherein the polypeptide members further comprise at least one of an N-terminal extension and a C-terminal extension of the CTLD.

28. The combinatorial library of claim 27, wherein the at least one of the N-terminal extension and C-terminal extension comprises polypeptides providing effector function, enzyme function, further binding function, or multimerizing function.

29. The combinatorial library of claim 27, wherein the at least one of the N-terminal extension and the C-terminal extension comprises the non-CTLD-portions of a native C-type lectin-like protein or C-type lectin or a C-type lectin lacking a functional transmembrane domain.

30. The combinatorial library of claim 29, wherein the proteins are multimers of a moiety comprising the CTLD.

31. A library of nucleic acid molecules encoding polypeptides of the combinatorial polypeptide library of claim 1.

32. The library of nucleic acid molecules of claim 31, wherein the nucleic acids molecules of the library are expressed in a display system, wherein the display system comprises an observable phenotype that represents at least one property of the displayed expression products and the corresponding genotypes.

33. A display system comprising the library of nucleic acid molecules of claim 31, wherein the display system is selected from a phage display system; a yeast display system; a viral display system; a cell-based display system; a ribosome-linked display system; and a plasmid-linked display system.

34. A method for generating the combinatorial library of claim 1 comprising creating any of Schemes (a)-(j) by generating at least one random mutation in at least one of the four loops in the LSA region of the CTLD.

35. The method of claim 34, wherein the at least one random mutation is created by oligonucleotide-directed randomization; DNA shuffling by random fragmentation; loop shuffling; loop walking; or error-prone PCR mutagenesis.

36. A method for identifying and isolating a polypeptide having specific binding activity to a target molecule, wherein the method comprises:
(a) providing a combinatorial polypeptide library of claim 1;
(b) contacting the combinatorial polypeptide library with the target molecule under conditions that allow for binding between a polypeptide and the target molecule; and
(c) isolating a polypeptide that binds to the target molecule.

37. The method of claim 36, wherein the method further comprises a library of nucleic acid molecules encoding polypeptides of the combinatorial polypeptide library, wherein the library of nucleic acids is expressed in a display system, and wherein the display system comprises an observable phenotype that represents at least one property of the displayed expression products and the corresponding genotypes.

38. A method for the identification and isolation of a polypeptide capable of specifically binding to a target molecule, said method comprising the steps of:
(a) providing a library of nucleic acid molecules encoding the polypeptide library of claim 1;
(b) expressing the library of nucleic acid molecules in a display system to obtain an ensemble of polypeptides, in which the amino acid residues at one or more sequence positions differ between different members of said ensemble of polypeptides;
(c) contacting the ensemble of polypeptides with said target molecule under conditions that allow for binding between a polypeptide and the target molecule; and
(d) isolating a polypeptide that is capable of binding to said target molecule.

39. A polypeptide having the scaffold structure of a C-type Lectin Like Domain (CTLD), wherein the polypeptide binds to a target other than a natural target for that CTLD, and wherein the CTLD scaffold structure of the CTLD is modified according to any of the schemes of claim 1.

40. The polypeptide of claim 39, wherein the polypeptide has the scaffold structure of the C-type Lectin Like Domain (CTLD) of human tetranectin and wherein the polypeptide binds to a target other than a natural target for human tetranectin.

41. A method for producing the polypeptide of claim 39, comprising contacting the combinatorial polypeptide library of claim 1 with the target molecule under conditions that allow for binding between the polypeptide and the target molecule and isolating a polypeptide that binds to the target molecule, wherein the target molecule is not the natural target for the CTLD.
